(19) United States
(12) Patent Application Publication
(10) Pub. No.: US 2019/0209472 A1
(43) Pub. Date: Jul. 11, 2019

(54) FORMULATIONS FOR DERMAL DELIVERY OF POLYMER CONJUGATES OF INDOLOCARBAZOLE COMPOUNDS WITH REDUCED EXPOSURE

(71) Applicant: Sienna Biopharmaceuticals, Inc., Westlake Village, CA (US)

(72) Inventors: Valentina Mainero, Turin (IT); Silvio Traversa, Turin (IT)

(21) Appl. No.: 16/208,278

(22) Filed: Dec. 3, 2018

Related U.S. Application Data
(60) Provisional application No. 62/594,428, filed on Dec. 4, 2017.
(60) Provisional application No. 62/632,676, filed on Feb. 20, 2018.
(60) Provisional application No. 62/702,273, filed on Jul. 23, 2018.
(60) Provisional application No. 62/723,347, filed on Aug. 27, 2018.
(60) Provisional application No. 62/732,538, filed on Sep. 17, 2018.

(51) Int. Cl. A61K 9/06; A61K 9/00; A61K 31/424; A61K 47/60; A61K 47/10; A61K 47/44; A61K 47/28; A61P 17/06

(52) U.S. Cl. CPC A61K 9/06; A61K 9/0014; A61K 31/424; A61P 17/06; A61K 47/10; A61K 47/44; A61K 47/28; A61K 47/60

(57) ABSTRACT

Topical formulations to treat skin conditions, such as psoriasis, are provided in which a polymer conjugate of an indolocarbazole exhibits reduced systemic exposure. An example formulation includes the TrkA antagonist, SNA-120, together with a preservative/antioxidant, a preservative/solvent, a solvent, a solvent/moisturizing agent, a stiffening agent, and one or more emollients/emulsifying agents.

### RELATED APPLICATIONS

This application claims priority to U.S. Provisional Patent Application No. 62/594,428, filed Dec. 4, 2017, U.S. Provisional Patent Application No. 62/632,676, filed Feb. 20, 2018, U.S. Provisional Patent Application No. 62/702,273, filed Jul. 23, 2018, U.S. Provisional Patent Application No. 62/723,347, filed Aug. 27, 2018, and U.S. Provisional Patent Application No. 62/732,538, filed Sep. 17, 2018, the entire content of each of which is incorporated herein by reference.

### BACKGROUND

Skin care formulations, which include for example, primers, serums, lotions, ointments, gels, creams, foams and other products, are used on the skin for various purposes. Formulations can contain one or more active agents, including in some embodiments, indolocarbazole compounds conjugated to polymer(s).

### SUMMARY

Formulations for dermal delivery of polymer conjugates of indolocarbazole compounds, having reduced exposure, are disclosed. In one example, the formulation includes: a polymer conjugate of an indolocarbazole compound, a preservative/antioxidant, a preservative/solvent, a solvent, a solvent/moisturizing agent, a stiffening agent, and one or more emollients/emulsifying agents.

In one embodiment, the polymer conjugate of an indolocarbazole compound is SNA-120. The SNA-120 may be present at about 0.005% to about 5% (w/w) of the formulation. In a variation, the SNA-120 is present at about 0.05% to about 0.5% (w/w) of the formulation (e.g., about 0.50%, 0.10%, or 0.050%).

In certain embodiments of the disclosed formulation, the preservative/antioxidant may be selected from natural antioxidants or synthetic antioxidants. The natural antioxidant may be selected from ascorbic acid or tocopherol. The synthetic antioxidant may be selected from propyl gallate, tertiary butylhydroquinone (TBHQ), butylated hydroxyanisole (BHA) or butylated hydroxytoluene (BHT). In one embodiment, the preservative/antioxidant is butylated hydroxytoluene (BHT). The preservative/antioxidant may be present at about 0.01% to about 1% (w/w) of the formulation. Alternatively, the preservative/antioxidant may be present at about 0.10% (w/w) of the formulation.

In certain embodiments of the disclosed formulation, the preservative/solvent may be selected from benzoic acid, sorbic acid, boric acid, methylparaben, ethylparaben, propylparaben, butylparaben, sodium benzoate, sodium propionate, potassium sorbate, chlorobutanol, benzyl alcohol and phenyl ethyl alcohol, phenol, chlorocresol, o-phenyl phenol, benzalkonium chloride, cetyl pyridinium chloride, imidurea, thimerisal, propyl gallate, butylated hydroxyanisole (BHA), butylated hydroxytoluene (BHT), citric acid or disodium edetate. In one embodiment, the preservative/solvent is benzyl alcohol. The preservative/solvent may be present at about 1% to about 10% (w/w) of the formulation. Alternatively, the preservative/solvent may be present at about 5% (w/w) of the formulation.

In certain embodiments of the disclosed formulation, the solvent may be selected from selected from ethanol, propylene glycol, glycerin or polyethylene glycol. In one embodiment, the solvent is propylene glycol. The solvent may be present at about 0.5% to about 20% (w/w) of the formulation. Alternatively, the solvent is present at about 5% to about 6% (w/w) of the formulation.

In certain embodiments of the disclosed formulation, the solvent/moisturizing agent may be selected from mineral oil, white petrolatum, stearyl alcohol, cetyl alcohol, isopropyl myristate, diisopropyl adipate, stearic acid or white wax. In one embodiment, the solvent/moisturizing agent is white petrolatum. The solvent/moisturizing agent may be present at about 10% to about 95% (w/w) of the formulation. Alternatively, the solvent/moisturizing agent may be present at about 75% (w/w) of the formulation.

In certain embodiments of the disclosed formulation, the stiffening agent may be selected from white wax, dimethicone or polymers. In one embodiment, the stiffening agent is white wax. The stiffening agent may be present at about 0.5% to about 20% (w/w) of the formulation. Alternatively, the stiffening agent may be present at about 8% (w/w) of the formulation.

In certain embodiments of the disclosed formulation, the one or more emollient/emulsifying agents may be selected from mineral oil, petrolatum, cholesterol, dimethicone, dimethiconol, stearyl alcohol, cetyl alcohol, behenyl alcohol, diisopropyl adipate, isopropyl myristate, myristyl myristate, cetyl ricinoleate, sorbitan distearate, sorbitan dilaurate, sorbitan stearate, sorbitan laurate, sucrose laurate, sucrose dilaurate, sodium isostearyl lactylate, lauryl pidolate, sorbitan stearate, PPG-14 butyl ether or PPG-15 stearyl ether. In one embodiment, two emollient/emulsifying agents are used. The combination of two emollient/emulsifying agents may consist of stearyl alcohol and cholesterol. The emollient/emulsifying agents may be present at about 0.5% to about 10% (w/w) of the formulation. Alternatively, both the stearyl alcohol and cholesterol are present at about 3% (w/w) of the formulation.

An ointment for dermal delivery of SNA-120 is disclosed in accordance with another embodiment. The ointment comprises: about 0.05% to about 0.5% w/w SNA-120, about 0.1% w/w butylated hydroxytoluene (BHT), about 5% w/w benzyl alcohol, about 5% to about 6% w/w propylene glycol, about 75% w/w white petrolatum, about 8% w/w white wax, about 3% w/w stearyl alcohol, and about 3% w/w cholesterol.

A method of treating a skin condition associated with TrkA signaling is disclosed in accordance with another embodiment. The method comprises: applying, or instructing application of, a topical formulation to a skin region, wherein the formulation inhibits TrkA signaling in the skin region, and thereby treats the skin condition, wherein the formulation comprises: a polymer conjugate of an indolocarbazole compound, a preservative/antioxidant, a preservative/solvent, a solvent, a solvent/moisturizing agent, a stiffening agent, and one or more emollients/emulsifying agents.

In a variation to the disclosed method, the polymer conjugate of an indolocarbazole may be SNA-120.

In one embodiment of the disclosed method, the skin condition is psoriasis. In another embodiment, the skin condition comprises pruritus associated with psoriasis or another dermatologic condition.

### BRIEF DESCRIPTION OF THE DRAWINGS

FIG. 1 depicts the dermal levels of CT327 (SNA-120) (calculated ng/cm2).

FIG. 2 depicts the epidermal levels of CT327 (SNA-120) (calculated ng/cm2).

FIG. 3 depicts combined dermal and epidermal levels of CT327 (SNA-120) (calculated ng/cm2).

FIG. 4A depicts the chemical structure of SNA-120.

FIG. 4B depicts the chemical structure of the degradation of SNA-120 to amide.

FIG. 5 depicts the shelf life prediction for batch PD11187 (0.05% w/w).

FIG. 6 depicts the shelf life prediction for batch PD11188 (0.1% w/w).

FIG. 7 depicts the shelf life prediction for batch PD11189 (0.5% w/w).

FIG. 8 depicts the shelf life prediction for batch PD11294 (0.05% w/w).

FIG. 9 depicts the shelf life prediction for batch PD12034 (0.05% w/w).

FIG. 10 depicts the shelf life prediction for batch PD11295 (0.1% w/w).

FIG. 11 depicts the shelf life prediction for batch PD11298 (0.1% w/w).

FIG. 12 depicts the shelf life prediction for batch PD11296 (0.5% w/w).

FIG. 13 depicts the shelf life prediction for batch PD11299 (0.5% w/w).

FIG. 14A depicts the shelf life prediction for a 0.05% w/w batch (data up to 60 months, Separate Slopes model).

FIG. 14B depicts the shelf life prediction for a 0.05% w/w batch (data up to 60 months, Separate Slopes model).

FIG. 14C depicts the shelf life prediction for a 0.05% w/w batch (data up to 60 months, Separate Slopes model).

FIG. 15 depicts the shelf life prediction for 0.1% w/w batches (data up to 60 months, Common Intercept and Common Slope model).

FIG. 16A depicts the shelf life prediction for a 0.5% w/w batch (data up to 68 months, Separate Intercepts and Common Slope model).

FIG. 16B depicts the shelf life prediction for a 0.5% w/w batch (data up to 68 months, Separate Intercepts and Common Slope model).

FIG. 16C depicts the shelf life prediction for a 0.5% w/w batch (data up to 68 months, Separate Intercepts and Common Slope model).

FIG. 17 depicts a flow diagram for the manufacture of a batch of SNA-120 ointment formulation.

FIG. 18A depicts a typical chromatogram of the lower limit of quantification with IS standard ([CT327]=5.00 ngml⁻¹, [CT327 amide]=2.00 ngml⁻¹).

FIG. 18B depicts a typical chromatogram of the upper limit of quantification with IS standard ([CT327]=250.10 ngml⁻¹, [CT327 amide]=100.06 ngml⁻¹).

FIG. 18C depicts a typical chromatogram of a blank sample (with an approximate RT of 3.5 min for CT327, 3.1 min for CT327 amide, and 4.8 min for IS).

FIG. 18D depicts a typical chromatogram of a patient day 1, 1 hour post-dose plasma sample (measured [CT327]=NQ, measured [CT327 amide]=NQ).

FIG. 19A depicts a typical chromatogram of the lower limit of quantification with IS standard ([CT327]=10.00 ngml⁻¹, [CT327 amide]=5.02 ngml⁻¹).

FIG. 19B depicts typical chromatograms of the upper limit of quantification with IS standard ([CT327]=500.20 ngml⁻¹, [CT327 amide]=251.11 ngml⁻¹).

FIG. 19C depicts typical chromatograms of a blank sample (with an approximate RT of 4.1 min for CT327, 3.0 min for CT327 amide, and 5.7 min for IS).

FIG. 19D depicts typical chromatograms of a patient day 3, pre-dose urine sample (measured [CT327]=NQ, measured [CT327 amide]=NQ).

### DETAILED DESCRIPTION

Effective delivery of pharmacologically active agents may be hindered by unwanted exposure of those agents to non-desired locations (such as the systemic circulation and/or lymphatic system). For example, topical agents useful in treating various skin disorders may result in toxic side effects because of systemic exposure. One issue with delivering compositions comprising one or more active agents topically (or non-topically) is the concern that such agents may need to be delivered in an amount and at a location sufficient to have a therapeutic effect. At the same time however, exposure (e.g., absorption or longevity of the composition in the systemic circulation, lymphatic system, or other non-targeted sites) may not be desirable for multiple reasons, including, but not limited to, safety reasons. There remains an unmet need for compounds with reduced exposure at non-target sites that result in a clinically therapeutic effect.

In several embodiments of the invention, the compositions described herein are both therapeutically efficacious and minimize non-target (e.g., systemic or bloodstream) exposure. In some embodiments, the active agents are PEGylated or otherwise coupled to large molecules, and surprisingly, are effective in crossing biological membranes such that the active agents are effectively delivered to the target location. Inflammatory and non-inflammatory conditions are contemplated herein.

Reduced exposure compounds and compositions are provided in several embodiments. “Reduced exposure” compounds are those compounds that, when delivered to a target location, are formulated to act at the target location with reduced exposure (e.g., entry and/or longevity) in non-target sites. Exposure is reduced as compared to active agents not formulated according to the embodiments described herein. As a non-limiting example, a PEGylated topical dermal active agent has reduced exposure to the bloodstream as compared to the active agent alone. Reduced exposure compounds include topical compounds that can be delivered to body surfaces and cavities such as the skin, eyes, ears, nose, mouth, vagina, rectum, etc. Non-desired target sites include, for example, the systemic system, the lymphatic system, non-target tissue, etc. “Reduced exposure compositions” comprise or consist essentially of one or more “reduced exposure compounds.”

Reduced exposure topical compositions are provided in many embodiments. In some cases, less or none of the active agent is absorbed into the non-target site (e.g., systemic circulation and/or lymphatic system). Further, once the composition enters the systemic circulation and/or lymphatic system, clearance (e.g., by the kidney) occurs at a much faster rate. One or more of the advantages of (i) reduced absorption into the non-target site (e.g., systemic circulation and/or lymphatic system), (ii) slower absorption into the non-target site (e.g., systemic circulation and/or lymphatic system), and (iii) faster clearance rates from the non-target site (e.g., systemic circulation and/or lymphatic system) are also achieved when using the compositions (e.g., via dermal topical formulations as described herein) for treating the skin.

In several embodiments, there is provided in a reduced exposure composition, a polymer conjugate comprising a warhead (e.g., at least one active agent) linked to a polymer, wherein the warhead comprises an indolocarbazole compound.

In several embodiments, there is provided in a reduced exposure composition, a polymer conjugate comprising a warhead (e.g., at least one active agent) linked to a polymer, wherein the warhead comprises an indolocarbazole compound. In some embodiments, the polymer conjugate comprises an indolocarbazole compound of formula (I):

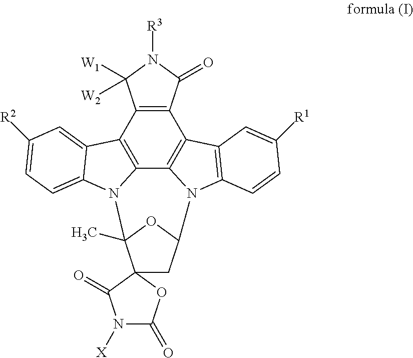

wherein R¹ and R² are the same or a different residue and are each independently selected from the group consisting of:

(a) hydrogen, halogen, substituted or unsubstituted lower alkyl, substituted or unsubstituted lower alkenyl, substituted or unsubstituted lower alkynyl, hydroxy, lower alkoxy, carboxy, lower alcoxycarbonyl, acyl, nitro, carbamoyl, lower alkylaminocarbonyl, —NR⁵R⁶, wherein R⁵ and R⁶ are each independently selected from hydrogen, substituted or unsubstituted lower alkyl, substituted or unsubstituted lower alkenyl, substituted or unsubstituted lower alkynyl, substituted or unsubstituted aryl, substituted or unsubstituted heteroaryl, substituted or unsubstituted aralkyl, substituted or unsubstituted lower alkylaminocarbonyl, substituted or unsubstituted lower arylaminocarbonyl, alkoxycarbonyl, carbamoyl, acyl or R⁵ and R⁶ are combined with a nitrogen atom to form a heterocyclic group,

(b) —CO(CH₂)ⱼR⁴, wherein j is 1 to 6, and R⁴ is selected from the group consisting of: (i) hydrogen, halogen, —N₃; (ii) —NR⁵R⁶, wherein R⁵ and R⁶ are as defined above; (iii) —SR⁷, wherein R⁷ is selected from the group consisting of hydrogen, substituted or unsubstituted lower alkyl, substituted or unsubstituted lower alkenyl, substituted or unsubstituted lower alkynyl, substituted or unsubstituted aryl, substituted or unsubstituted heteroaryl, substituted or unsubstituted aralkyl, —(CH₂)ₐCO₂R¹⁰ (wherein a is 1 or 2, and wherein R¹⁰ is selected from the group consisting of hydrogen and substituted or unsubstituted lower alkyl) and —(CH₂)ₐCO₂NR⁵R⁶; and (iv) —OR⁸, —OCOR⁸, wherein R⁸ is selected from hydrogen, substituted or unsubstituted lower alkyl, substituted or unsubstituted lower alkenyl, substituted or unsubstituted lower alkynyl, substituted or unsubstituted aryl, substituted or unsubstituted heteroaryl

(c) —CH(OH)(CH₂)ⱼR⁴ wherein j and R⁴ are as defined above;

(d) —(CH₂)_{d}CHR¹¹CO₂R¹² or —(CH₂)_{d}CHR¹¹CONR⁵R⁶, wherein d is 0 to 5, is hydrogen, —CONR⁵R⁶, or —CO₂R¹³, wherein R¹³ is hydrogen or a wherein substituted or unsubstituted lower alkyl, and R¹² is hydrogen or a substituted or unsubstituted lower alkyl;

(e) —(CH₂)ₖR¹⁴ wherein k is 2 to 6 and R¹⁴ is halogen, substituted or unsubstituted aryl, substituted or unsubstituted heteroaryl, —COOR¹⁵, —OR¹⁵, (wherein R¹⁵ is hydrogen, substituted or unsubstituted lower alkyl, substituted or unsubstituted lower alkenyl, substituted or unsubstituted lower alkynyl, substituted or unsubstituted aryl, substituted or unsubstituted heteroaryl or acyl), —SR⁷ (wherein R⁷ is as defined above), —CONR⁵R⁶, —NR⁵R⁶ (wherein R⁵ and R⁶ are as defined above) or —N₃;

(f) —CH═CH(CH₂).R¹⁶, wherein m is 0 to 4, and R¹⁶ is hydrogen, substituted or unsubstituted lower alkyl, substituted or unsubstituted lower alkenyl, substituted or unsubstituted lower alkynyl, substituted or unsubstituted aryl, substituted or unsubstituted heteroaryl, —COOR¹⁵, —OR¹⁵ (wherein R¹⁵ is as defined above) —CONR⁵R⁶ or —NR⁵R⁶ (wherein R⁵ and R⁶ are as defined above);

(g) —CH═C(CO₂R¹²)₂, wherein R¹² is as defined above;

(h) —C≡C(CH2)nR16, wherein n is 0 to 4 and R16 is as defined above;

(i) —CH₂OR²², wherein R²² is tri-lower alkyl silyl in which the three lower alkyl groups are the same or different or wherein R²² has the same meaning as R⁸;

(j) —CH(SR²³)₂ and —CH₂—SR⁷ wherein R²³ is lower alkyl, lower alkenyl or lower alkynyl and wherein R⁷ is as defined above; and

R₃ is hydrogen, halogen, acyl, carbamoyl, substituted or unsubstituted lower alkyl, substituted or unsubstituted alkenyl, substituted or unsubstituted lower alkynyl or amino; and

W¹ and W² are independently hydrogen, hydroxy or W¹ and W² together represent oxygen; and

X is a polymer moiety, either linear or branched,

or a pharmaceutically acceptable salt of formula (I).

The polymer moiety X is covalently attached to the indolocarbazole compound of formula (I) may be biocompatible, can be of natural or semi-synthetic or synthetic origin and can have a linear or branched structure. In some embodiments, the polymer moiety X is selected from poly(alkylene oxides), in particular from (polyethylene) oxides. However, further exemplary polymers include without limitation polyacrylic acid, polyacrylates, polyacrylamide or N-alkyl derivatives thereof, polymethacrylic acid, polymethacrylates, polyethylacrylic acid, polyethylacrylates, polyvinylpyrrolidone, poly(vinylalcohol), polyglycolic acid, polylactic acid, poly(lactic-co-glycolic) acid, dextran, chitosan, polyaminoacids, hydroxyethyl starch.

In some embodiments, the polymer moiety X is a polyethylene glycol (PEG) moiety, wherein the terminal OH group can optionally be modified e.g. with C₁-C₅ alkyl or C₁-C₅ acyl groups. In some embodiments, the terminal OH group is optionally modified with C₁-, C₂- or C₃-alkyl groups or C₁-, C₂- or C₃ groups. In some embodiments, the modified polyethylene glycol is a terminally alkoxy-substituted polyethylene glycol. In some embodiments, the polymer moiety is methoxy-polyethylene-glycol (mPEG).

As used in this application, except as otherwise expressly provided herein, each of the following terms shall have the meaning set forth below.

As used herein, the term “about” will be understood by one of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise. As used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. The use of “or” or “and” means “and/or” unless the context indicates otherwise. Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The term “lower alkyl”, when used alone or in combination with other groups, means a straight chained or branched lower alkyl group containing from 1-6 carbon atoms, preferably from 1-5, more preferably from 1-4 and especially preferably 1-3 or 1-2 carbon atoms. These groups include, in some embodiments, methyl, ethyl, propyl, isopropyl, butyl, isobutyl, sec-butyl, tert-butyl, pentyl, amyl, isoamyl, neopentyl, 1-ethylpropyl, hexyl, and the like. The lower alkyl moiety of the “lower alkoxy”, the “lower alkoxycarbonyl”, the “lower akylaminocarbonyl”, “lower hydroxyalkyl” and of the “tri-lower alkylsilyl” groups has the same meaning as “lower alkyl” defined above.

The “lower alkenyl” groups are defined as C₂-C₆ alkenyl groups which may be straight chained or branched and may be in the Z or E form. Such groups include vinyl, propenyl, 1-butenyl, isobutenyl, 2-butenyl, 1-pentenyl, (Z)-2-pentenyl, (E)-2-pentenyl, (Z)-4-methyl-2-pentenyl, (E)-4-methyl-2-pentenyl, pentadienyl, e.g., 1, 3 or 2,4-pentadienyl, and the like. In some embodiments, the C₂-C₆-alkenyl groups are C₂-C₅-, C₂-C₄-alkenyl groups. In other embodiments, the C₂-C₆-alkenyl groups are C₂-C₃-alkenyl groups.

The term “lower alkynyl” groups refers to C₂-C₆-alkynyl groups which may be straight chained or branched and include ethynyl, propynyl, 1-butynyl, 2-butynyl, 1-pentynyl, 2-pentynyl, 3-methyl-1-pentynyl, 3-pentynyl, 1-hexynyl, 2-hexynyl, 3-hexynyl and the like. In some embodiments, C₂-C₆-alkynyl groups are C₂-C₅—, C₂-C₄-alkynyl groups. In other embodiments, C₂-C₆-alkynyl groups are C₂-C₃-alkynyl groups.

The term “aryl” group refers to C₆-C₁₄-aryl groups which contain from 6 up to 14 ring carbon atoms. These groups may be mono-, bi- or tricyclic and are fused rings. In some embodiments, the aryl groups include phenyl, biphenyl, naphthyl, anthracenyl, phenanthrenyl and the like. The aryl moiety of the “arylcarbonyl” and the “arylaminocarbonyl” groups has the same meaning as defined above.

The term “heteroaryl” groups may contain 1 to 3 heteroatoms independently selected from nitrogen, sulfur or oxygen and refers C₃-C₁₃-heteroaryl groups. These groups may be mono-, bi- or tricyclic. In some embodiments, the C₃-C₁₃ heteroaryl groups include heteroaromatics and saturated and partially saturated heterocyclic groups. These heterocyclics may be monocyclic, bicyclic, tricyclic. In some embodiments, the 5 or 6-membered heterocyclic groups are thienyl, furyl, pyrrolyl, pyridyl, pyranyl, morpholinyl, pyrazinyl, methyl pyrrolyl, and pyridazinyl. The C₃-C₁₃-heteroaryl may be a bicyclic heterocyclic group. In some embodiments, the bicyclic heterocyclic groups are benzofuryl, benzothienyl, indolyl, imidazolyl, and pyrimidinyl. In some embodiments, the C₃-C₁₃-heteroaryls are furyl and pyridyl.

The term “lower alkoxy” includes alkoxy groups containing from 1 to 6 carbon atoms, in some embodiments from 1 to 5, in other embodiments from 1-4 and in yet other embodiments 1 to 3 or 1 to 2 carbon atoms and may be straight chained or branched. These groups include methoxy, ethoxy, propoxy, butoxy, isopropoxy, tert-butoxy, pentoxy, hexoxy and the like.

The term “acyl” includes lower alkanoyl containing 1 to 6 carbon atoms, in some embodiments from 1 to 5, from 1 to 4, from 1 to 3 or from 1 to 2 carbon atoms and may be straight chained or branched. These groups include, in some embodiments, formyl, acetyl, propionyl, butyryl, isobutyryl, tertiary butyryl, pentanoyl and hexanoyl. The acyl moiety of the “acyloxy” group has the same meaning as defined above.

The term “halogen” includes fluoro, chloro, bromo, iodio, and the like.

The term “aralkyl” group refers C₇-C₁₅-aralkyl wherein the alkyl group is substituted by an aryl. The alkyl group and aryl may be selected from the C₁-C₆ alkyl groups and the C₆-C₁₄-aryl groups as defined above, wherein the total number of carbon atoms is between 7 and 15. In some embodiments the C₇-C₁₅-aralkyl groups are benzyl, phenylethyl, phenylpropyl, phenylisopropyl, phenylbutyl, diphenylmethyl, 1,1-diphenylethyl, 1,2-diphenylethyl. The aralkyl moiety of the “aralkyloxy” groups has the same meaning as defined above.

The substituted lower alkyl, alkenyl and alkynyl groups have 1 to 3 independently selected substituents, such as lower alkyl, hydroxy, lower alkoxy, carboxyl, lower alkoxycarbonyl, nitro, halogen, amino, mono- or di-lower alkylamino, dioxolane, dioxane, dithiolane, and dithione. The lower alkyl substituent moiety of the substituted lower alkyl, alkenyl and alkynyl groups, and the lower alkyl moiety of the lower alkoxy, the lower alkoxycarbonyl, and the mono- or di-lower alkylamino substituents of the substituted lower alkyl, alkenyl and alkynyl groups have the same meaning as “lower alkyl” defined above.

The substituted aryl, the substituted heteroaryl and the substituted aralkyl groups each has 1 to 3 independently selected substituents, such as lower alkyl, hydroxy, lower alkoxy, carboxy, lower alkoxycarbonyl, nitro, amino, mono- or di-lower alkylamino, and halogen. The lower alkyl moiety of the lower alkyl, the lower alkoxy, the lower alkoxycarbonyl, and the mono- or di-lower alkylamino groups among the substituents has the same meaning as ‘lower alkyl’ defined above.

The heterocyclic group formed by R⁵ and R⁶ combined with a nitrogen atom includes pyrrolidinyl, piperidinyl, piperidino, morpholinyl, morpholino, thiomorpholino, N-methylpiperazinyl, indolyl, and isoindolyl.

In some embodiments, R¹ and R² are independently selected from the group consisting of hydrogen, halogen, nitro, —CH₂OH, —(CH₂)ₖR¹⁴, —CH═CH(CH₂)ₘR¹⁶, —C≡C(CH₂)ₘR¹⁵, —CO(CH₂)ⱼR⁴ wherein R⁴ is —SR⁷, CH₂O-(substituted or unsubstituted) lower alkyl (wherein the substituted lower alkyl is in some embodiments methoxymethyl, methoxyethyl or ethoxymethyl), —NR⁵R⁶. In some embodiments, each of R¹ and R² is hydrogen.

In some embodiments of R¹ and R², the residue R¹⁴ is selected from phenyl, pyridyl, imidazolyl, thiazolyl, tetrazolyl, —COOR¹⁵, —OR¹⁵ (wherein R¹⁵ is in some embodiments selected from hydrogen, methyl, ethyl, phenyl or acyl), —SR⁷ (wherein R⁷ is in some embodiments selected from substituted or unsubstituted lower alkyl, 2-thiazoline and pyridyl) and —NR⁵R⁶(wherein R⁵ and R⁶ are in some embodiments selected from hydrogen, methyl, ethyl, phenyl, carbamoyl and lower alkylaminocarbonyl). Moreover, in some embodiments, the residue R¹⁶ is selected from hydrogen, methyl, ethyl, phenyl, imidazole, thiazole, tetrazole, —COOR¹⁵, —OR¹⁵ and —NR⁵R⁶ (wherein the residues R¹⁵, R⁵ and R⁶ have the meanings as described above). In some embodiments of R¹ and R², the residue R⁷ is selected from the group consisting of substituted or unsubstituted lower alkyl, substituted or unsubstituted phenyl, pyridyl, pyrimidinyl, thiazole and tetrazole. Further, in some embodiments, k is 2, 3 or 4, j is 1 or 2 and m and n are independently 0 or 1.

In some embodiments, R³ is hydrogen or acetyl. Furthermore, in some embodiments, each W¹ and W² is hydrogen.

In some embodiments, the warhead of the polymer conjugate is a derivative of K252a, which has the formula:

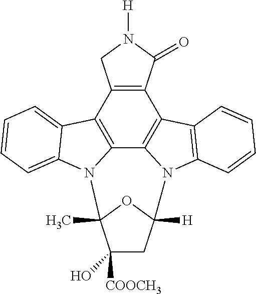 [SMILES: COC(=O)[C@]1(O)C[C@@H]2O[C@@]1(C)n1c3ccccc3c3c4c(c5c6ccccc6n2c5c31)C(=O)NC4]

In some embodiments, the polymer conjugate is SNA-120, wherein the composition has the formula:

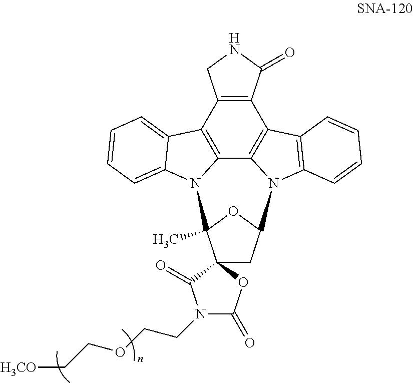

The formulas depicted herein are not limited to any particular stereochemistry, and all stereoisomers and enantiomers thereof are included in this disclosure.

As described above, several embodiments disclosed herein provide reduced or minimized exposure (e.g., entry into and/or longevity in a non-target site such as the systemic circulation and/or lymphatic system). In some embodiments, exposure at a non-target site is less than 90%, 75%, 50%, 25%, 15%, 10%, 5% or 2% (or less) of the polymer conjugate as compared to a similar active entity that has not been produced according to the embodiments described herein. In some embodiments, desirable rate of clearance from the non-target site (e.g., systemic circulation and/or lymphatic system) for the compositions described herein is increased by at least 10%, 25%, 50%, or 75% or more as compared to non-conjugated controls. As an example, a PEGylated active agent described herein not only penetrates the desired membranes to reach a desired target, but has reduced non-target exposure by at least 20-80% or more as compared to the non-PEGylated active agent. In some embodiments, blood concentrations measured post administration of the compositions described herein are less than about 0.1 ng/ml, less than 1 ng/ml, or less than 10 ng/ml after, e.g., 15 minutes, 30 minutes, 1 hour, 6 hours or 12 hours.

In some embodiments, reduced exposure at non-target sites contributes to enhanced efficacy. Efficacy may be enhanced because lower concentrations/amounts/dosing schedules are required to achieve the same or similar therapeutic efficacy at the target site (because, for example, the active ingredient stays at the desired target site for a longer time). In one embodiment, concentrations/amounts/dosing schedules are reduced by 25%-75% or more.

More rapid clearance rates of the active agent once in the non-target site(s) (such as systemic circulation and/or lymphatic system) are also beneficial because this may allow for a higher concentration or more doses to be delivered. This is especially beneficial for active agents in which a subject would benefit from a higher dose but cannot tolerate the higher dose due to toxicity at the non-target site (e.g., systemic toxicity). Faster clearance rates would permit the desired higher dose to be delivered according to the desired schedule. For example, a subject may be able to tolerate daily doses rather than weekly doses because of the reduced exposure.

In some embodiments, the active agents of the compositions described herein (e.g., indolocarbazole compounds conjugated e.g., with PEG or other polymers) are measured in non-target sites (e.g., the systemic circulation and/or lymphatic system) at less than amounts found when the active agent is delivered without conjugation (e.g., less than 0.5%, 1% or 2% after 6 or 12 hours, as compared with 3-15% (e.g., 3-6%) when the active agent is delivered without conjugation). In some embodiments, the active agents of the compositions described herein (e.g., indolocarbazole compounds conjugated e.g., with PEG or other polymers) are measured in non-target sites (e.g., the systemic circulation and/or lymphatic system) at less than 0.5%, 1% or 2% after 3-24 hours, as compared to an amount 2-20 times greater when the active agent is delivered without conjugation.

In some embodiments, clearance of the compositions (e.g., the conjugated polymer compounds) occurs within minutes of exposure to the non-target site (e.g., systemic circulation and/or lymphatic system), as opposed to hours. In other embodiments, 50% clearance of the conjugated polymer compounds occurs in less than 5 minutes, 15 minutes, 30 minutes, 1 hour, 6 hours, and 12 hours of exposure to the systemic circulation and/or lymphatic system. Clearance times of the conjugated polymer compounds are reduced by more than 25%, 50%, 75% and 90%, as compared to the non-conjugated active agents or other formulations. These reduced clearance times are beneficial to reduce toxicity and undesired side effects.

In some embodiments, an active agent may be increasingly toxic as it is metabolized in the non-target site (e.g., systemic circulation and/or lymphatic system) because the metabolites exhibit more toxicity than the original agent. Thus, faster clearance rates, in some cases even before the toxic metabolites are created, are especially beneficial.

The term “active entity” or “active agent” as used herein should not be understood as limiting the participation of the polymer itself and/or the chemical linking moiety between the polymer and the warhead in defining the pharmacology of the polymer conjugate. In some embodiments, the polymer influences the selectivity and/or inhibitory activity of the polymer conjugate. In some embodiments, the chemical linking moiety between the polymer and warhead influences the selectivity and/or inhibitory activity of the polymer conjugate. In some embodiments, the polymer conjugates exhibit no change in selectivity or inhibitory activity against the therapeutic target in comparison with the unconjugated active agent. In some embodiments, the polymer conjugates exhibit a significant increase in selectivity against the therapeutic target in comparison with the unconjugated active agent. In some embodiments, the polymer conjugates exhibit a significant increase in inhibitory activity against the therapeutic target in comparison with the unconjugated active agent. In some embodiments, the polymer conjugates exhibit a significant increase in selectivity and inhibitory activity against the therapeutic target in comparison with the unconjugated active agent. In some embodiments, the increased selectivity and/or inhibitory activity of the polymer conjugate against the therapeutic target in comparison with the unconjugated active agent causes decrease in undesired biological effects. In some embodiments, the increased selectivity of the polymer conjugate is caused by an increase of the hydrodynamic volume resulting from the conjugated polymer chain. In some embodiments, the polymer chain creates a higher steric hindrance which allows discrimination among the diverse shapes and sizes of the binding sites of different proteins, thus improving selectivity with respect to the active agent alone.

In several embodiments, various inflammatory skin diseases are treated (e.g., treated by administering an effective amount of a formulation provided in Table 1). The inflammatory skin disease comprises, in some embodiments, psoriasis, psoriasis guttata, inverse psoriasis, pustular psoriasis, psoriatic erythroderma, acute febrile neutrophilic dermatosis, eczema, xerotic eczema, dyshidrotic eczema, vesicular palmar eczema, acne vulgaris, atopic dermatitis, contact dermatitis, allergic contact dermatitis, dermatomyositis, exfoliative dermatitis, hand eczema, pompholyx, keloids, rosacea, rosacea due to sarcoidosis, rosacea due to scleroderma, rosacea due to Sweet syndrome, rosacea due to systemic lupus erythematosus, rosacea due to urticaria, rosacea due to herpetic pain, Sweet's disease, neutrophilic hydrodenitis, sterile pustule, drug rash, seborrheic dermatitis, pityriasis rosea, Kikuchi's disease of the skin, pruritic urticarial papules and plaques of pregnancy, Stevens-Johnson syndrome and toxic epidermal necrolysis, tattoo reaction, Wells syndrome (eosinophilic cellulitis), reactive arthritis (Reiter syndrome), bowel-associated dermatosis-arthritis syndrome, rheumatoid neutrophilic dermatosis, neutrophilic eccrine hidradenitis, neutrophilic skin disease of dorsum of hand, balanitis circumscripta plasmacellularis, balanoposthitis, Behcet's disease, erythema annulare centrifugum, erythema dyschromicum perstans, erythema multiforme, granuloma annulare, dermatitis of hand, lichen nitidus, lichen planus, lichen sclerosus et atrophicus, lichen simplex chronicus, lichen spinulosus, nummular dermatitis, pyoderma gangrenosum, sarcoidosis, subcorneal pustular dermatosis, urticaria, and transient acantholytic dermatosis.

In some embodiments, provided herein are methods of treating psoriasis (e.g., psoriatic plaques) in a subject in need thereof, comprising administering an effective amount of a formulation provided in Table 1 to the subject.

In some embodiments, provided herein are methods of treating atopic dermatitis in a subject in need thereof, comprising administering an effective amount of a formulation provided in Table 1 to the subject.

In some embodiments, provided herein are methods of treating pruritis (e.g., pruritis associated with psoriasis or atopic dermatitis) in a subject in need thereof, comprising administering an effective amount of a formulation provided in Table 1 to the subject.

In some embodiments, provided herein are methods of treating psoriasis (e.g., psoriatic plaques) in a subject in need thereof, comprising administering an effective amount of a formulation comprising about 0.05% to about 0.5 w/w SNA-120 (e.g., about 0.05% or about 0.5 w/w) to the subject.

In some embodiments, provided herein are methods of treating atopic dermatitis in a subject in need thereof, comprising administering an effective amount of a formulation comprising about 0.05% to about 0.5% w/w SNA-120 (e.g., about 0.05% or about 0.5% w/w) to the subject.

In some embodiments, provided herein are methods of treating pruritis (e.g., pruritis associated with psoriasis or atopic dermatitis) in a subject in need thereof, comprising administering an effective amount of a formulation comprising about 0.05% to about 0.5% w/w SNA-120 (e.g., about 0.05% or about 0.5% w/w) to the subject.

In some embodiments, provided herein are methods of treating psoriasis (e.g., psoriatic plaques) in a subject in need thereof, comprising administering an effective amount of a formulation comprising butylated hydroxy toluene, benzyl alcohol, propylene glycol, white petrolatum, white wax, stearyl alcohol, and cholesterol (e.g., about 0.10% w/w butylated hydroxy toluene, 5.00 w/w benzyl alcohol, 5.40 w/w propylene glycol, 75.00% w/w, white petrolatum, 8.00% w/w white wax, 3.00% w/w stearyl alcohol, and 3.00% w/w cholesterol) to the subject.

In some embodiments, provided herein are methods of treating atopic dermatitis in a subject in need thereof, comprising administering an effective amount of a formulation comprising butylated hydroxy toluene, benzyl alcohol, propylene glycol, white petrolatum, white wax, stearyl alcohol, and cholesterol (e.g., about 0.10% w/w butylated hydroxy toluene, 5.00% w/w benzyl alcohol, 5.40% w/w propylene glycol, 75.00% w/w, white petrolatum, 8.00% w/w white wax, 3.00% w/w stearyl alcohol, and 3.00% w/w cholesterol) to the subject.

In some embodiments, provided herein are methods of treating pruritis (e.g., pruritis associated with psoriasis or atopic dermatitis) in a subject in need thereof, comprising administering an effective amount of a formulation comprising butylated hydroxy toluene, benzyl alcohol, propylene glycol, white petrolatum, white wax, stearyl alcohol, and cholesterol (e.g., about 0.10% w/w butylated hydroxy toluene, 5.00% w/w benzyl alcohol, 5.40% w/w propylene glycol, 75.00% w/w, white petrolatum, 8.00% w/w white wax, 3.00% w/w stearyl alcohol, and 3.00% w/w cholesterol) to the subject.

In some embodiments, the administration is once daily (e.g., once daily for 8 weeks or 12 weeks). In some embodiments, the administration is twice daily (e.g., twice daily for 8 weeks or 12 weeks).

In some embodiments, the subject has a weekly mean I-NRS score of at least 5 on the 11 point I-NRS scale.

In some embodiments, the subject has a weekly mean I-NRS score of at least 5 on the 11 point I-NRS scale and the psoriasis is mild to moderate psoriasis (e.g., mild to moderate plaque psoriasis).

In some embodiments, the subject has mild-to-moderate psoriasis with at least moderate itch.

In some embodiments, the psoriasis is mild to moderate psoriasis.

In some embodiments, the psoriasis is mild to moderate plaque psoriasis.

In several embodiments, various skin neoplasias are treated (e.g., treated by administering an effective amount of a formulation provided in Table 1). The skin neoplasia comprises, in some embodiments, squamous cell carcinoma, basal cell carcinoma, malignant melanoma, malignant cutaneous lymphoma, Kaposi's sarcoma, Merkel cell skin cancer, and non-melanoma skin cancer.

In several embodiments, various vascular tumors are treated (e.g., treated by administering an effective amount of a formulation provided in Table 1). The vascular tumor comprises, in some embodiments, hemangiomas, Kaposi's sarcoma, lymphangioma, glomangioma, angiosarcoma, hemangioendothelioma, and infantile hemangiomas.

In several embodiments, various bullous diseases are treated (e.g., treated by administering an effective amount of a formulation provided in Table 1). The bullous disease comprises, in some embodiments, bullous pemphigoid, erythema multiforme, dermatitis herpetiformis, epidermolysis bullosa acquisita, linear Immunoglobulin A disease, mucous membrane pemphigoid, pemphigoid gestationis, pemphigus foliaceus, and pemphigus vulgaris.

In several embodiments, hair growth and cycling are modulated (e.g., modulated by administering an effective amount of a formulation provided in Table 1). In several embodiments, alopecia is treated.

In several embodiments, the polymer conjugates (e.g., a formulation provided in Table 1) are administered in combination with UV irradiation therapy.

Also provided herein, in several embodiments, are polymer conjugates wherein the polymer is polyethylene glycol (PEG) or methoxy-polyethylene glycol (m-PEG). In several embodiments, there is provided a pharmaceutical composition comprising or consisting essentially of a polymer conjugate disclosed herein that is formulated for topical administration. In several embodiments, methods of making and using the compositions described herein are provided.

In several embodiments, the invention comprises a reduced exposure composition comprising at least one active entity linked to at least one polymer, wherein the composition has reduced exposure at a non-target site as compared to the active entity delivered without the polymer. The non-target site comprises the systemic system, the lymphatic system and/or another non-target tissue site in some embodiments.

Local peripheral nerves in the skin play an important role in the pathogenesis of psoriasis, as the absence of neural input has been shown to lead to plaque clearance. Plaques in psoriasis patients with associated itch have elevated levels of NGF-immunoreactive keratinocytes and TrkA expression in innervating nerve fibers. The NGF/TrkA signaling pathway is important in neurogenic inflammation and keratinocyte hyperproliferation which contribute to psoriatic plaques, as well as itch. Without being bound by theory, SNA-120 is a topical tropomyosin receptor kinase A (TrkA) inhibitor that blocks nerve growth factor (NGF) signaling (i.e. SNA-120 selectively targets the NGF/TrkA signaling pathway), which plays an important role in the pathogenesis of psoriasis and itch.

In some embodiments, the active entity binds to a tropomyosin-receptor-kinase A (TrkA) in some embodiments. The active entity binds to a Janus Kinase (JAK) family member in some embodiments. The active entity binds to one or more of Janus Kinase 1 (JAK1), Janus Kinase 2 (JAK2), Janus Kinase 3 (JAK3), and/or Tyrosine kinase 2 (TYK2) in some embodiments. The active entity binds to mitogen-activated protein kinase kinase (MAP2K) in some embodiments. The active entity binds to mitogen-activated protein kinase kinase 3 (MAP2K3) in some embodiments. The binding may be partially or fully inhibitory or not.

In some embodiments, the polymer used in the reduced exposure compounds comprises polyethylene glycol (PEG) and/or methoxy-polyethylene glycol (m-PEG). In embodiments where the active entity has one or more carboxyl, hydroxyl, amino and/or sulfhydryl groups, the active entity is PEGylated (or conjugated/coupled to another polymer) at one or more of said carboxyl, hydroxyl, amino and/or sulfhydryl groups.

The reduced exposure compositions described herein are formulated for topical administration in several embodiments. In several embodiments, methods of treating one or more of the following are provided: inflammatory skin disease, vascular tumors, skin neoplasia, bullous diseases, alopecia, wounds, scars, autoimmune disorders, and cancerous or pre-cancerous lesions. Methods for modulating hair growth and cycling are provided in some embodiments.

In one embodiment, a method of treating any of the above-mentioned skin diseases or any other skin condition in need of treatment, includes: applying, or instructing application of, a topical formulation (e.g., a formulation provided in Table 1) to a skin region, wherein the formulation fully or partially inhibits signaling (e.g., NGF/TrkA signaling pathway) in the skin region, and thereby treats the skin condition, wherein the formulation comprises: a polymer conjugate of an indolocarbazole compound in an oil-in-water emulsion. The topical formulation applied in accordance with the method may be any of the ointment formulations of SNA-120 as more fully described herein (e.g., a formulation of Table 1).

In some embodiments, the compositions provided herein (e.g., a formulation or placebo of Table 1) may be administered via at least two routes of administration, either simultaneously or sequentially according to some embodiments. In one embodiment, the composition is administered via a first (e.g. topical dermal) route to a subject, wherein the subject further receives an additional agent via a second (e.g., non-dermal) route to achieve synergetic effects.

### Formulations for Topical Delivery

In some embodiments, the active agent is formulated for topical delivery. The active agent may be a reduced exposure composition. In certain embodiments, the active agent is a polymer conjugate of an indolocarbazole compound, thereby providing a reduced exposure indolocarbazole compound. In one particular embodiment, the polymer conjugate is the indolocabazole depicted in Formula (1). In another particular embodiment, the polymer conjugate is SNA-120 (formerly referred to as CT327; both terms are used herein interchangeably). The topical delivery formulation can be in any form, including for example, an ointment, a gel, a balm, a cream, a lotion, a primer, a serum, a liquid, a spray, etc.

The weight percentage (w/w) of active agent to the total weight of the topical delivery formulation may range from about 0.001% to about 10%, from about 0.005% to about 5%, from about 0.01% to about 1%, or from about 0.05% to about 5%, including any weight percentages within the disclosed ranges. In other embodiments, the weight percentage (w/w) of the active agent may be greater than or equal to about 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99% or greater than or equal to about 1.00% w/w. In certain embodiments, the weight percentage (w/w) of the active agent to the total weight of the topical delivery formulation may fall within any range defined by any two of the above weight percentages.

In other embodiments, the weight percentage (w/w) of the active agent may be less than or equal to about 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02% or less than or equal to about 0.01% w/w.

In several embodiments, the active agent may be formulated with one or more excipients selected from a preservative, an antioxidant, a solvent, an oil phase component/moisturizing agent, a stiffening agent, an emollient, an emulsifying agent. Non-limiting examples of compounds that may be used as preservatives and that also function as antioxidants (referred to also as “preservative/antioxidant”) include: natural antioxidants such as ascorbic acid and tocopherols, as well as synthetic antioxidants such as propyl gallate, tertiary butylhydroquinone (TBHQ), butylated hydroxyanisole (BHA) and butylated hydroxytoluene (BHT). The weight percentage of the preservative/antioxidant may vary from about 0.01 to about 1 percent (w/w). In some embodiments, the weight percentage of the preservative/antioxidant may be about 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99% or about 1.00% w/w. In certain embodiments, the weight percentage (w/w) of the preservative/antioxidant to the total weight of the topical delivery formulation may fall within any range defined by any two of the above weight percentages. In some embodiments, the weight percentage of the preservative/antioxidant may be greater than about 0.001% or less than about 10% w/w. In certain embodiments of the topical formulation, the preservative/antioxidant is butylated hydroxytoluene (BHT), which is present in a weight percentage of about 0.1% w/w.

In some embodiments, another preservative (besides the preservative/antioxidant disclosed above) may be included in the formulation. The additional preservative may also function as a solvent for the topical formulation (referred to also as “preservative/solvent”). Non-limiting examples of compounds that may be used as preservative/solvent include: acids such as benzoic acid, sorbic acids and boric acids, esters such as methylparaben, ethylparaben, propylparaben, butylparaben, sodium benzoate, sodium propionate and potassium sorbate, alcohols such as chlorobutanol, benzyl alcohol and phenyl ethyl alcohol, phenols such as phenol, chlorocresol, o-phenyl phenol, and quaternary ammonium compounds such as benzalkonium chloride and cetyl pyridinium chloride. Common preservatives in topical formulations include: methylparaben, propylparaben, butylparaben, benzyl alcohol, sorbic acid, imidurea, thimerisal, propyl gallate, BHA, BHT, citric acid, disodium edetate, and the like. The weight percentage of the preservative/solvent may vary from about 0.5 to about 20 percent (w/w). In some embodiments, the weight percentage of the preservative may be about 0.50%, 0.60%, 0.70%, 0.80%, 0.90%, 1.00%, 1.50%, 2.00%, 2.50%, 3.00%, 3.50%, 4.00%, 4.50%, 5.00%, 5.50%, 6.00%, 6.50%, 7.00%, 7.50%, 8.00%, 8.50%, 9.00%, 9.50%, 10.00%, 11%, 12%, 13%, 14%, 15%, 16%, 17%, 18%, 19% or about 20% w/w. In certain embodiments, the weight percentage (w/w) of the preservative/solvent to the total weight of the topical delivery formulation may fall within any range defined by any two of the above weight percentages. In some embodiments, the weight percentage of the preservative/solvent may be greater than about 0.05% or less than about 50% w/w. In certain embodiments of the topical formulation, the preservative/solvent is benzyl alcohol, which is present in a weight percentage of about 5.0% w/w.

In some embodiments, a solvent may be included in the formulation. The solvent may be a single component or a mixture. The solvent may be a water-miscible solvent (i.e. a cosolvent). Such water-miscible cosolvents may be used to assist in dissolving the active agent (together, the single or mixed solvents, cosolvents and water-miscible solves are referred to also as simply “solvent”). Examples of solvents include those that are miscible with water such as ethanol, propylene glycol, glycerin, polyethylene glycol 400, and the like. Certain solvents, such as glycerin or propylene glycol, also add beneficial humectant properties to the composition. Drug delivery and penetration into the skin can be modified by the water-miscible cosolvent composition. The weight percentage of the solvent may vary from about 0.5 to about 20 percent (w/w). In some embodiments, the weight percentage of the water-miscible cosolvent may be about 0.50%, 0.60%, 0.70%, 0.80%, 0.90%, 1.00%, 1.50%, 2.00%, 2.50%, 3.00%, 3.50%, 4.00%, 4.50%, 5.00%, 5.50%, 6.00%, 6.50%, 7.00%, 7.50%, 8.00%, 8.50%, 9.00%, 9.50%, 10.00%, 11%, 12%, 13%, 14%, 15%, 16%, 17%, 18%, 19% or about 20% w/w. In certain embodiments, the weight percentage (w/w) of the solvent to the total weight of the topical delivery formulation may fall within any range defined by any two of the above weight percentages. In some embodiments, the weight percentage of the solvent may be greater than about 0.05% or less than about 50% w/w. In certain embodiments of the topical formulation, the solvent is propylene glycol, which is present in a weight percentage of about 5.0% to 6% w/w. The weight percentage of the solvent may vary with the weight percentage of active agent.

In some embodiments, a solvent that also functions as a moisturizing agent may be included in the formulation (referred to also as “solvent/moisturizing agent”). Oil phase components together with emulsifying agents may be used as solvents/moisturizing agents. Oil phase components that are commonly used in the art include mineral oil, white petrolatum, stearyl alcohol, cetyl alcohol, isopropyl myristate, diisopropyl adipate, stearic acid, white wax, and the like. In certain embodiments of the topical formulation, an oil phase component, such as white petrolatum is used at weight percentages (w/w) that vary between about 10% to about 95%. In some embodiments, the weight percentage of the solvent/moisturizing agent may be about 10%, 15%, 20%, 25%, 30%, 35%, 40%, 45%, 50%, 55%, 60%, 65%, 70%, 75%, 80%, 85%, 90% or about 95% w/w. In certain embodiments, the weight percentage (w/w) of the solvent/moisturizing agent to the total weight of the topical delivery formulation may be any amount that falls within any range defined by any two of the above weight percentages. In some embodiments, the weight percentage of the solvent/moisturizing agent may be greater than about 50% or less than about 90% w/w. In certain embodiments of the topical formulation, the solvent/moisturizing agent is white petrolatum, which is present in a weight percentage of about 75% w/w.

In some embodiments, a thickening or stiffening agent may be employed to increase the viscosity of the topical formulation (referred to as “stiffening agent”). Typical stiffening agents include white wax, dimethicone and polymers. The weight percentage of the stiffening agent, such as white wax, may vary from about 0.5 to about 20 percent (w/w). In some embodiments, the weight percentage of the stiffening agent may be about 0.50%, 0.60%, 0.70%, 0.80%, 0.90%, 1.00%, 1.50%, 2.00%, 2.50%, 3.00%, 3.50%, 4.00%, 4.50%, 5.00%, 5.50%, 6.00%, 6.50%, 7.00%, 7.50%, 8.00%, 8.50%, 9.00%, 9.50%, 10.00%, 11%, 12%, 13%, 14%, 15%, 16%, 17%, 18%, 19% or about 20% w/w. In certain embodiments, the weight percentage (w/w) of the stiffening agent to the total weight of the topical delivery formulation may fall within any range defined by any two of the above weight percentages. In some embodiments, the weight percentage of the stiffening agent may be greater than about 0.05% or less than about 50% w/w. In certain embodiments of the topical formulation, the stiffening agent is white wax, which is present in a weight percentage of about 8% w/w.

One or more emollients and/or emulsifying agents may be included in the topical formulation (referred to as “emollient/emulsifying agent”). Typical emollient/emulsifying agents include mineral oil, petrolatum, cholesterol, dimethicone, dimethiconol, stearyl alcohol, cetyl alcohol, behenyl alcohol, diisopropyl adipate, isopropyl myristate, myristyl myristate, cetyl ricinoleate, sorbitan distearate, sorbitan dilaurate, sorbitan stearate, sorbitan laurate, sucrose laurate, sucrose dilaurate, sodium isostearyl lactylate, lauryl pidolate, sorbitan stearate, PPG-14 butyl ether, PPG-15 stearyl ether, and mixtures thereof. The weight percentage of the emollient/emulsifying agent may vary from about 0.1 to about 20 percent (w/w). In some embodiments, the weight percentage of the emollient/emulsifying agent may be about 0.50%, 0.60%, 0.70%, 0.80%, 0.90%, 1.00%, 1.50%, 2.00%, 2.50%, 3.00%, 3.50%, 4.00%, 4.50%, 5.00%, 5.50%, 6.00%, 6.50%, 7.00%, 7.50%, 8.00%, 8.50%, 9.00%, 9.50% or about 10.00% w/w. In certain embodiments, the weight percentage (w/w) of the emollient/emulsifying agent to the total weight of the topical delivery formulation may fall within any range defined by any two of the above weight percentages. In some embodiments, the weight percentage of the emollient/emulsifying agent may be greater than about 0.05% or less than about 50% w/w. In certain embodiments, the topical formulation includes one, two, three or four different emollient/emulsifying agents. In one embodiment, two agents, stearyl alcohol and cholesterol, are used at weight percentages of about 3% w/w each agent.

### SNA-120 Formulation

Table 1 provides the composition of one SNA-120 ointment formulation at different concentrations (as well as a placebo).

TABLE 1
SNA-120 OINTMENT FORMULATION

| | 0.5% | 0.1% | 0.05% | | |
|---|---|---|---|---|---|
| Component | w/w | w/w | w/w | Placebo | Function |
| BHT (Butylated | 0.10 | 0.10 | 0.10 | 0.10 | Preservative/ |
| Hydroxy Toluene) | | | | | Antioxidant |
| Benzyl Alcohol | 5.00 | 5.00 | 5.00 | 5.00 | Preservative/ |
| | | | | | Solvent |
| Propylene Glycol | 5.40 | 5.80 | 5.85 | 5.90 | Solvent |
| White Petrolatum | 75.00 | 75.00 | 75.00 | 75.00 | Solvent/Mois- |
| | | | | | turizing agent |
| White Wax | 8.00 | 8.00 | 8.00 | 8.00 | Stiffening agent |
| Stearyl Alcohol | 3.00 | 3.00 | 3.00 | 3.00 | Emollient/ |
| | | | | | Emulsifying agent |
| Cholesterol | 3.00 | 3.00 | 3.00 | 3.00 | Emollient/ |
| | | | | | Emulsifying agent |
| SNA-120 | 0.50 | 0.10 | 0.05 | — | Active |
| | | | | | Pharmaceutical |
| | | | | | Ingredient |

### Example 1: SNA-120 (CT327) Formulation Development

### Introduction

Initial formulation development of a topical drug product containing CT327 (0.1 and 0.5%) for treatment of psoriasis and/or atopic dermatitis was undertaken. The overall objective was to develop a chemically stable, anhydrous topical product which could be stored at room temperature. All considered excipients were selected from FDA's Inactive Ingredients database.

In the early stages of pre-formulation several product and formulation concepts were considered. The goal of these compositions was to investigate the stability of CT327 in solubilized (dissolved) form in various solvent blends at room temperature. Data generated during this preliminary feasibility evaluation was utilized to develop four different formulation approaches viz. 1) petrolatum-based ointments, 2) polar gels, 3) PEG ointments and 4) non-polar gels.

Based on physical and chemical stability data and other factors, a petrolatum-based ointment and a polar gel were selected for further consideration. Ultimately a petroleum-based ointment was chosen to advance as the lead candidate.

This Example summarizes the development history, formulation design, stability of the various formulation prototypes and ultimate selection of a lead candidate.

### Drug Substance

Drug name: CT327 (SNA-120)

Chemical name: (9S, 10R, 12R)-10-(3′-mPEG₂₀₀₀-1′,3′-oxazolidine-2′,4′-dione)-2,3,9,10,11,12-hexahydro-9-methyl-1-oxo-9,12-epoxy-1H-diindolo[1,2,3-fg:3′,2′,1′-k,l]pyrrolo[3,4-i][1,6] benzodiazocine]

Physicochemical Properties

Molecular weight: 2353 (on average calculation)

Physical Form: White—pale yellow—yellow solid

Molecular Formula: C₃₂H₂₈&N₄O₇.[C₂H₄O]ₙ centered on ₙ=40

Solubility: Extremely soluble in ethanol, benzyl alcohol and water

Stability

Exposure to artificial daylight fluorescent lamp causes degradation. A progressive increase in degradation rate observed with increase in temperature from room temperature to 40° C. to 60° C. CT327 is susceptible to oxidative stress. Neutral and alkaline pH causes degradation.

### Preformulation—Solubility Evaluation of CT327

Purpose

Previously acquired stability data indicated that CT327 possessed poor aqueous stability. Therefore, solubility screening focused on anhydrous solvents. The purpose of the solubility study was to identify appropriate solvent(s) and/or solvent blend(s) suitable for the development of an anhydrous topical dosage form.

Solvent selection was based on inclusion in the FDA's Inactive Ingredients Guide (IIG) for approved topical products. Twenty-one solvents were selected for the evaluation. Solubility was assessed visually.

Methodology

Under dim light conditions a small amount of API (˜0.05 g) was weighed into type 1 clear glass vials. To each vial, approximately 10 g of the solvent was added. Samples were shaken manually for a few minutes and visually observed to check if the API had dissolved, as indicated by the clarity of the solution. In the event API dissolved, the procedure was repeated by adding more API up to a maximum of 10% w/w. If the API did not dissolve, samples were sonicated and observed periodically to check for solubility.

Results

Table 2 summarizes the solubility of CT327 in single solvents along with their corresponding IIG concentration. Except when noted the IIG concentration stated is the maximum approved in topical products.

TABLE 2
SOLUBILITY OF CT327 IN SINGLE SOLVENTS

| | | | IIG |
|---|---|---|---|
| | Results | | concentration |
| Solvents | % w/w | Comments | (% w/w) |
| Ethanol (190 proof) | ≥10% | Excellent solubility | 96.7 |
| Benzyl alcohol | ≥10% | Excellent solubility | 50 |
| Lactic acid | ≥10% | Excellent solubility | 18.6 |
| Benzyl benzoate | ≥10% | Excellent solubility | 46 (IM) |
| N-Methyl | ≥10% | Excellent solubility | 50 (Periodontal) |
| pyrrolidone | | | |
| Propylene | ≥10% | Excellent solubility | 5 |
| carbonate | | | |
| Dimethyl | ≥5% | Requires some | 15 |
| isosorbide | | sonication (15-30 | |
| | | minutes) to dissolve | |
| Transcutol | ≥2% | Requires some | 25 |
| | | sonication (15-30 | |
| | | minutes) to dissolve | |
| Propylene | ~1% | Requires prolonged | 98.1 |
| glycol | | sonication (>2 hrs) | |
| PEG 400 | ~0.5% | Requires prolonged | 69.9 |
| | | sonication (>2 hrs) | |
| Cyclomethicone | <0.5% | Does not dissolve | 13 |
| | | completely even after | |
| | | prolonged sonication | |
| Diisopropyl | <0.5% | Dissolves after | 20 |
| adipate | | prolonged sonication, | |
| | | precipitates after 24 | |
| | | hours | |
| Oleyl alcohol | <0.5% | Does not dissolve | 10 |
| | | completely even after | |
| | | prolonged sonication | |
| Hexylene glycol | <0.5% | Does not dissolve | 12 |
| | | completely even after | |
| | | prolonged sonication | |
| Isostearyl | <0.5% | Does not dissolve | 25 |
| alcohol | | completely even after | |
| | | prolonged sonication | |
| Isostearic acid | <0.5% | Does not dissolve | 25 |
| | | completely even after | |
| | | prolonged sonication | |
| Caprylic/capric | <0.5% | Does not dissolve | 50 |
| triglycerides | | completely even after | |
| | | prolonged sonication | |
| Octyldodecanol | <0.5% | Does not dissolve | 12 |
| | | completely even after | |
| | | prolonged sonication | |
| Isopropyl | <0.5% | Does not dissolve | 35 |
| myristate | | completely even after | |
| | | prolonged sonication | |
| Myristyl | <0.5% | Hard to detect | 92.8 |
| lactate | | solubility, forms white | |
| | | cloudy solution after | |
| | | some sonication | |
| Diethyl | <0.5% | Hard to detect | 24 |
| sebacate | | solubility, forms white | |
| | | cloudy solution after | |
| | | some sonication | |

### Conclusion

Benzyl alcohol, ethanol, propylene carbonate and benzyl benzoate were identified as primary solvents as the solubility of CT327 in these solvents was ≥10% w/w. N-methyl pyrrolidone, in which CT327 showed good solubility, was not considered for further development because of skin irritation concerns for these indications. Lactic acid was eliminated because of its high water content and a potential cause for concern for stability of the final product.

### Preformulation—Compatibility of CT327 in Solution and Solvent Blends

Purpose

The purpose of this study was to evaluate the compatibility of CT327 with: a) primary solvents and b) combination of primary solvents and anhydrous excipients. This was done to identify the solvents and excipients which could be included in prototype formulations.

Study Design

### Compatibility of CT327 in Single Solvents

The compatibility of CT327 in primary solvents i.e. benzyl alcohol, ethanol (190 proof), propylene carbonate and benzyl benzoate was determined by preparing 0.5% w/w CT327 solutions in each of these solvents. Aliquots of these solutions were transferred into foil-covered type I clear glass vials, and stored at 5° C., 25° C. and 40° C. for 3 months. Table 2 describes the samples evaluated.

### Compatibility of CT327 in Solvent Blends

Other excipients for this study were selected on the basis of the following criteria: 1) miscibility with primary solvents; and 2) no adverse effect on the solubility of CT327.

CT327 solutions at 0.5% w/w in solvent blends were prepared by first dissolving CT327 in the primary solvent then adding and mixing it with other excipients. Aliquots of the solutions were stored in foil-covered type I glass vials at 5° C., 25° C. and 40° C. for 3 months. Details of the composition of the test samples are provided in Tables 3 and 4.

### Testing Conditions

For samples stored at 25° C. and 40° C., testing included assay and appearance, and was conducted at T=0, 2 weeks, 1 month and 3 months. For samples stored at 5° C. only appearance was monitored.

TABLE 3
SINGLE SOLVENTS EVALUATED WITH CT327, 0.5% W/W

| Lot No | Primary solvent |
|---|---|
| 3136-20C | Ethanol (190 proof) |
| 3136-15A | Benzyl alcohol |
| 3136-13A | Propylene carbonate |
| 3136-15B | Benzyl benzoate |

TABLE 4
SOLVENT BLENDS USED TO EVALUATE
COMPATIBILITY OF CT327

| Lot No | Solvent blends |
|---|---|
| 3136-18A | Ethanol, 190 proof (10%) + CCT (50%) + IPM (40%) |
| 3136-28A | Ethanol, 190 proof (10%) + IPM (90%) |
| 3136-21B | Ethanol, 190 proof (10%) + CCT (50%) + IPM (25%) + |
| | cyclomethicone (15%) |
| 3136-18B | Ethanol, 190 proof (10%) + CCT (50%) + Isostearyl alcohol |
| | (40%) |
| 3136-19A | Ethanol, 190 proof (10%) + CCT (50%) + Isostearic acid |
| | (40%) |
| 3136-19B | Ethanol, 190 proof (10%) + CCT (50%) + Diisopropyl adipate |
| | (40%) |
| 3136-19C | Ethanol, 190 proof (10%) + CCT (50%) + Diethyl sebacate |
| | (40%) |
| 3136-22A | Ethanol, 190 proof (10%) + PEG 400 (60%) + PEG 200 |
| | (30%) |
| 3136-27A | Ethanol, 190 proof (99%) + PEG 400 (60%) + PEG 200 |
| | (30%) + BHT (0.1%) |
| 3136-20B | Ethanol, 190 proof (10%) + PEG 400 SR (90%) |
| 3136-16A | Ethanol, 190 proof (10%) + PEG 400 SR (60%) + Transcutol |
| | (30%) |
| 3136-17A | Ethanol, 190 proof (10%) + PEG 400 SR (70%) + Dimethyl |
| | isosorbide (20%) |
| 136-20A | Ethanol, 190 proof (10%) 4-propylene glycol (90%) |
| 3136-20D | Ethanol, 190 proof (10%) + propylene glycol (90%) + |
| | Transcutol (30%) |
| 3136-21A | Ethanol, 190 proof (10%) + propylene glycol (90%) + |
| | Dimethyl isosorbide (20%) |
| 3136-26A | Ethanol, 190 proof (9.9%) + propylene glycol (90%) + |
| | Transcutol (30%) + BHT (0.1%) |
| 3136-26B | Ethanol, 190 proof (10%) + propylene glycol (90%) + |
| | Dimethyl isosorbide (20%) + BHT (01%) |
| 3136-13C | Propylene carbonate (5%) + PEG 400 SR (95%) |
| 3136-14B | Propylene carbonate (5%) + PEG 400 SR (65%) + Transcutol |
| | (30%) |
| 3136-14A | Propylene carbonate (5%) + PEG 400 SR (75%) + Dimethyl |
| | isosorbide (2.0%) |
| 3136-13B | Propylene carbonate (5%) + propylene glycol (95%) |

CCT—Capric/caprylic triglycerides
IPM—Isopropyl myristate
SR—Super-refined

Results and Discussion

The stability of 0.5% CT327 solutions in solvent blends was investigated at different time points. At each time point, samples that showed poor physical and/or chemical stability were discontinued from the study.

### Two Weeks

Tables 5 & 6 summarize the results of physical and chemical testing of 0.5% CT327 solutions and solvent blends after being stored at 5° C., 25° C. and 40° C. for 2 weeks. The following six observations were made.

First, CT327 solutions in primary solvents (3136-20C, 15A, 13A and I SB) were physically stable (no change in color, lack of precipitation). After 2 weeks at 40° C. only solutions in propylene carbonate and benzyl benzoate show no significant change in drug concentration.

Second, CT327 solutions in ethanol and non-polar solvent blends containing CCT, IPM, cyclomethicone and isostearic acid (3136-18A, 28A, 21B, 19A, 19B, 19C) showed good physical stability but these samples, except the one with isostearic acid, could not be analyzed due to analytical extraction issues.

Third, in a majority of CT327 solutions in ethanol and polar solvent blends; PEG, propylene glycol, transcutol & dimethyl isosorbide (3136-20A, 20D, 21A, 26A, 26B), white precipitate was observed at 5° C., indicating solubility issues during refrigeration. These solutions remained clear and colorless at 2S° C. and 40° C.

Fourth, CT327 solutions in propylene carbonate and polar solvent blends: PEG, propylene glycol, transcutol & dimethyl isosorbide, also showed precipitation at 5° C. Blends with propylene carbonate and PEG (super-refined) showed yellow coloration.

Fifth, upon comparing CT327 solutions (in ethanol and PEG blends), 3136-22A, 27A and 20B, it was observed that solutions containing BHT showed better chemical stability. A similar trend was observed when comparing assay values of samples 3136-21A & 26B and 3136-20D with 3136-26 A.

Sixth, CT327 solution in ethanol+propylene glycol showed no significant change in assay (3136-20A) after 2 weeks at 40° C. CT327 solution in propylene carbonate+propylene glycol also showed no significant change in the drug assay values.

Samples 3136-13A, 19A, 27A, 26A, 26B, 13B (highlighted in Tables 5 & 6, appeared to be compatible with CT327 as no significant (≥4%) drop in assay was reported after 2 weeks at 40° C. These samples were subjected to physical and chemical testing at 1 and 3 months, while testing of the other samples was discontinued.

The comparison of assay results of 0.5% CT327 samples in PEG and propylene glycol formulations with and without BHT indicates that BHT may improve chemical stability of these samples. Hence PEG and propylene glycol samples without BHT were not tested after the 2-week time point.

TABLE 5
APPEARANCE OF 0.5% CT327 SOLUTIONS
IN SINGLE SOLVENTS AND SOLVENT
BLENDS AT TIME ZERO AND 2 WEEKS

| | Time | 2 weeks | | |
|---|---|---|---|---|
| Lot No | Zero | 44° C. | 25° C. | 5° C. |
| 3136-20C | C, cl | C, cl | C, cl | C, cl |
| 3136-15A | C, cl | C, cl | C, cl | C, cl |
| 3136-13A | C, cl | C, cl | C, cl | C, cl |
| 3136-15B | C, cl | C, cl | C, cl | C, cl |
| 3136-18A | C, cl | C, cl | C, cl | C, cl. |
| 3136-28A | C, cl | C, cl | C, cl | C, cl |
| 3136-21B | C, cl | C, cl | C, cl | C, cl |
| 3136-18B | C, cl | Clear, | White precipitate | White precipitate |
| | | yellow | | |
| 3136-19A | C, cl | C, cl | C, cl | C, cl |
| 3136-19B | Cloudy, | Hazy, | C, cl | White precipitate |
| | white | colorless | | |
| 3136-19C | Cloudy, | C, cl | C, cl | White precipitate |
| | white | | | |
| 3136-22A | C, cl | Clear, | White precipitate | White precipitate |
| | | yellow | | |
| 3136-27A | C, cl | C, cl | C, cl | White precipitate |
| 3136-20B | C, cl | Clear, | C, cl | Cloudy, |
| | | yellow | | white |
| 3136-16A | C, cl | C, cl | C, cl | Cloudy, |
| | | | | white |
| 3136-17A | C, cl | Clear, | C, cl | C, cl |
| | | yellow | | |
| 3136-20A | C, cl | C, cl | C, cl | White precipitate |
| 3136-20D | C, cl | C, cl | C, cl | White precipitate |
| 3136-21A | C, cl | Clear, | C, cl | White precipitate |
| | | yellow | | |
| 3136-26A | C, cl | C, cl | C, cl | White precipitate |
| 3136-26B | C, cl | C, cl | C, cl | White precipitate |
| 3136-13C | C, cl | Clear, | White precipitate | White precipitate |
| | | yellow | | |
| 3136-14B | C, cl | Clear, | C, cl | White precipitate |
| | | yellow | | |
| 3136-14A | C, cl | Clear, | Clear, | White precipitate |
| | | yellow | yellow | |
| 3136-13B | C, cl | C, cl | C, cl | White precipitate |

Highlighted appearance results of samples that showed good compatibility with CT327
C, cl—clear, colorless

TABLE 6
ASSAY RESULTS OF 0.5% CT327 SOLUTIONS
IN SINGLE SOLVENTS AND SOLVENT BLENDS
AT TIME ZERO AND 2 WEEKS

| | | |
|---|---|---|
| | Time Zero | 2 weeks, 40° C. |
| | % w/w | % w/w |
| Lot No | (individual) Average | (individual) Average |

| 3136-20C | (103.2, 103.1) | 103.1 | (85.7, 84.9) | 85.3 |
|---|---|---|---|---|
| 3136-15A | (100.5, 100.5) | 100.5 | (90.4, 90.7) | 90.5 |
| 3136-13A | (101.7, 101.9) | 101.8 | (100.8, 100.9) | 100.9 |
| 3136-15B | (88.0, 87.6) | 87.8 | (88.7, 89.1) | 88.9 |
| 3136-18A | (1.1, 1.0) | 1 | None detected | |
| 3136-28A | (7.4, 7.0) | 7.2 | (5.8, 13.4) | 9.6 |
| 3136-21B | (1.3, 1.3) | 1.3 | None detected | |
| 3136-18B | (56.0, 55.4) | 55.7 | (73.4, 72.1) | 72.8 |
| 3136-19A | (101.7, 101.7) | 101.7 | (97.8, 98.3) | 98 |
| 3136-19B | (66.9, 67.0) | 66.9 | (66.0, 65.8) | 65.9 |
| 3136-19C | (40.5, 53.1) | 46.8 | (39.1, 38.6) | 38.8 |
| 3136-22A | (102.3, 103.6) | 102.9 | (97.0, 98.1) | 97.6 |
| 3136-27A | (100.3, 99.5) | 99.9 | (100.0, 99.7) | 99.9 |
| 3136-20B | (99.8, 99.3) | 99.5 | (86.8, 86.0) | 86.4 |
| 3136-16A | (99.8, 99.7) | 99.8 | (96.9, 94.4) | 95.7 |
| 3136-17A | (95.4, 95.6) | 95.5 | (86.6, 86.6) | 86.6 |
| 3136-20A | (98.4, 97.9) | 98.2 | (97.1, 96.0) | 96.5 |
| 3136-20D | (97.5, 98.5) | 98 | (96.3, 96.0) | 96.2 |
| 3136-21A | (99.4, 101.0) | 100.2 | (94.0, 93.8) | 93.9 |
| 3136-26A | (94.2, 99.4) | 96.8 | (97.4, 98.1) | 97.7 |
| 3136-26B | (99.8, 100.2) | 100 | (97.9, 97.5) | 97.7 |
| 3136-13C | (104.7, 98.7) | 97.2 | (81.8, 73.0) | 77.4 |
| 3136-14B | 100.4, 100.4) | 100.4 | (86.8, 88.6) | 87.7 |
| 3136-14A | (97.5, 97.8) | 97.6 | (71.6, 71.4) | 71.5 |
| 3136-13B | (99.5, 99.6) | 99.5 | (98.9, 98.9) | 98.9 |

Highlighted assay results of samples that showed good compatibility with CT327

### 1 Month and 3 Months

The results of physical and chemical testing of CT327 solutions after being stored at 5° C., 25° C. and 40° C. for a period of 3 months are provided in Tables 7 & 8.

The 3 month, 25° C. result for 3136-27A is much lower than expected and also lower than 40° C. result. Upon closer observation, a small amount of a white precipitate was observed in the 25° C. 3-month sample, which could explain the lower assay value.

The 3 month, 25° C. result for 3136-13B is also lower than expected and also lower than 40° C. result. No precipitation was observed at 25° C. The sample was re-analyzed, but showed no significant change in the assay value. No further investigation was conducted, since promising alternatives were available.

TABLE 7
APPEARANCE OF 0.5% CT327 SOLUTIONS IN SINGLE SOLVENTS
AND SOLVENT BLENDS AT 1 AND 3 MONTHS

| | 1 Month | | | 3 Months | | |
|---|---|---|---|---|---|---|
| Lot No | 40° C. | 25° C. | 5° C. | 40° C. | 25° C. | 5° C. |
| 3136-13A | Clear, | Clear, | Clear, | Clear, | Clear, | Clear, |
| [propylene | colorless | colorless | colorless | colorless | colorless | colorless |
| carbonate] | | | | | | |
| 3136-19A | Clear, | Clear, | Clear, | Clear, | Clear, | Clear, |
| [EtOH + CCT + | colorless | colorless | colorless | colorless | colorless | colorless |
| isostearic acid] | | | | | | |
| 3136-27A | Clear, | Clear, | White | Clear, | White** | White |
| [EtOH + PEG 4t − | colorless | colorless | precipitate | colorless | precipitate | precipitate |
| PEG 200 + | | | | | | |
| BHT] | | | | | | |
| 3136-26A | Clear, | Clear, | White | Clear, | Clear, | White |
| [EtOH + PG + | colorless | colorless | precipitate | colorless | colorless | precipitate |
| transcutol + | | | | | | |
| BHT] | | | | | | |
| 3136-26B | Clear, | Clear, | White | Clear, | Clear, | White |
| [EtOH + PG + | colorless | colorless | precipitate | colorless | colorless | precipitate |
| dimethyl | | | | | | |
| isosorbide + | | | | | | |
| BHT] | | | | | | |
| 3136-13B | Clear, | Clear, | White | Clear, | Clear, | White |
| [propylene | colorless | colorless | precipitate | colorless | colorless | precipitate |
| carbonate + PG] | | | | | | |

**In sample 3136-27A stored at 25° C. for 3 months small white specks were observed, while in other samples where “white precipitate” is noted, the presence of the precipitates was readily evident.
EtOH: ethanol, 190 proof
PG: propylene glycol
CCT: caprylickapric triglyccrides

TABLE 8
ASSAY RESULTS OF 0.5% CT327 SOLUTIONS IN SINGLE
SOLVENTS AND SOLVENT BLENDS AT 1 AND 3 MONTHS

| | 1 Month | | 3 Months | |
|---|---|---|---|---|
| | 40° C. % w/w | 25° C. % w/w | 40° C. % w/w | 25° C. % w/w |
| | (individual) | (individual) | (individual) | (individual) |
| Lot No | Average | Average | Average | Average |
| 3136-13A | (101.5, 101) | (101.5, 101.1) | (100.1, 100) | (100.1, 100.0) |
| [propylene carbonate] | 101.2 | 101.3 | 100.1 | 100.0 |
| 3136-19A | (95.4, 95.3) | (85.9, 95.6) | (94.3, 94.6) | (98.8, 98.8) |
| [EtOH + CCT + | 95.4 | 90.8 | 94.5 | 98.7 |
| isostearic acid] | | | | |
| 3136-27A | (91.3, 91.0) | (97.0, 98.1) | (92.1, 93.8) | (82.6, 82.8) |
| [EOM + PEG 400 + | 91.1 | 97.6 | 92.9 | 82.7 |
| PEG 200 + BHT] | | | | |
| 3136-26A | (100.0, 99.6) | (100.2, 100.1) | (97.4, 97.2) | (98.5, 98.4) |
| [EtOH + PG + | 99.8 | 100.1 | 97.3 | 98.4 |
| transcutol + BHT | | | | |
| 3136-268 | (100.4, 100.5) | (100.4, 100.1) | (98.5, 98.2) | (98.8, 98.8) |
| [Et011 + PG + | 100.4 | 100.3 | 98.4 | 98.8 |
| dimethyl isosorbide + | | | | |
| BHT] | | | | |
| 3136-138 | (98.9, 99.6) | (98.7, 98.9) | (98.5, 98.5) | (86.6, 86.2) |
| [propylene | 99.2 | 98.8 | 98.5 | 86.4 |
| carbonate + PG] | | | | |

Summary

Ethanol and propylene carbonate were selected as primary solvents for CT327. Compatibility with key excipients, such as PEG, propylene glycol, 8HT, etc was established. This information was used to design formulation prototypes for CT327 as discussed in the next section.

### Development of Prototypes

Formulation Design Approaches

Based on the results of pre-formulation studies four formulation approaches were considered: 1) petrolatum based ointments; 2) polar gels; 3) PEG ointments; and 4) non-polar gels.

### Petrolatum Based Ointments

It was observed that CT327 has excellent solubility and stability in propylene carbonate. CT327 in blends containing propylene carbonate and propylene glycol also showed good stability. In topical applications, propylene carbonate has been used in combination with propylene glycol as a solvent for corticosteroids. The corticosteroid is dissolved in the solvent mixture to yield microdroplets that can then be dispersed in petrolatum. Hence a petrolatum-based ointment approach with the drug dissolved in a suitable solvent blend was considered for formulation prototype development.

### Polar Gels

CT327 in blends of alcohol, propylene glycol, Transcutol and BHT, CT327 showed good solubility and chemical stability. This solvent blend was utilized to develop a polar gel prototype.

### PEG Ointments

During pre-formulation studies it was observed that CT327 solutions in PEG containing BHT, showed acceptable stability. Analysis of the chemical assay results indicated an increase in amide degradation product. It was hypothesized that adding an acidic component such as citric acid or lactic acid could potentially reduce the rate of degradation. Hence PEG ointment prototypes, containing lactic acid or citric acid, were considered for further evaluation.

### Non-Polar Gel

CT327 in blends of ethanol and non-polar solvents such as caprylic/capric triglycerides and isostearic acid showed relatively good chemical and physical stability. However, since isostearic add is not compendial, it was not included in the prototypes. CT327 in blends of ethanol and non-polar solvents such as caprylic/capric triglyceride s, cyclomethicone and isopropyl myristate also showed good physical stability. This formed the basis of a non-polar gel approach.

### Formulation Components

It was recommended the antioxidant BHT be used in all formulation prototypes. Original formulation design included 3 petrolatum based and 2 PEG based ointments all of which had propylene carbonate as primary solvent. Since propylene carbonate was not available in USP-NF grade, it was replaced by benzyl alcohol in the petrolatum and PEG ointment prototypes. Benzyl alcohol had earlier been identified as a good solvent for CT327.

Formulation Feasibility and Placebo Prototypes

To minimize the use of the available API, initial formulation development was conducted with the corresponding placebos. It was assumed that low concentration of the API in the target product, 0.5%, would not affect the physical stability of the prototype. Placebo compositions of the proposed prototypes were prepared and the appearance and physical stability of the ointments and gels was assessed.

### Petrolatum Based Ointments

Three placebo prototypes: (A) 3136-40, (B) 3136-51, and (C) 3136-46, were prepared. Their compositions are provided in Table 9. Samples were packaged in clear glass vials and stored at ambient conditions and under accelerated conditions of 40° C. and 50° C. After 2 weeks at 40° C. and 50° C. no changes in physical stability such as discoloration or liquid separation were observed. These three different types of petrolatum prototypes were included in an R&D stability study. Note that a change was made in prototype B (3136-51): propylene carbonate was replaced with benzyl alcohol, since the former was not available in commercial grade.

TABLE 9
FORMULATION COMPOSITIONS OF PETROLATUM
BASED PLACEBO OINTMENTS

| | 3136-40 | 3136-51 | 3136-46 |
|---|---|---|---|
| | Prototype A | Prototype B | Prototype C |
| Ingredients | (% w/w) | | |
| CT327 | NA | NA | NA |
| Propylene Carbonate | 5.0 | 5.0 | 5.0 |
| Propylene Glycol | 75 | 5.4 | — |
| Mineral Oil | — | — | 12.0 |
| Stearyl Alcohol | | 3.0 | — |
| Cetyl Alcohol | 2.0 | — | _{—} |
| Polyoxyethylene (2) | 2.0 | — | — |
| Stearyl Ether (Brij 72) | | | |
| Cholesterol | — | 3.0 | — |
| Butylated Hydroxytoluene | 0.1 | 0.1 | 0.1 |
| (BHT) | | | |
| White Petrolatum | 83.3 | 75.5 | 79.9 |
| White Wax | — | 8.0 | |
| Paraffin Wax Pastilles | — | — | 3.0 |

### Polar Gels

Two gelling agents; hydroxypropyl cellulose (HPC or Klucel™) and Carbopol 980 were evaluated. Compositions are provided in Table 10. A cloudy gel was observed with Carbopol 980, while the use of Klucel resulted in a clear gel. Klucel was selected as the gelling agent. Formulation composition of 3136-48 was selected as the base for the polar gels.

Dimethyl isosorbide had been included in the initial formulations. Since this ingredient was not available as USP NF grade, it was removed and the concentration of ethanol was increased to 30%. These gels were stored at ambient conditions and at 40° C. for 2 weeks. No liquid separation was observed.

TABLE 10
FORMULATION COMPOSITIONS
OF POLAR PLACEBO GELS

| | | 3136-47-B | 3136-48 |
|---|---|---|---|
| Ingredients | | (% w/w) | |
| 1 | CT327 | — | — |
| 2 | Propylene Glycol | 37.90 | 38.0 |
| 3 | Benzyl Alcohol | 5.0 | 5.0 |
| 4 | Propylene Carbonate | 5.0 | 5.0 |
| 5 | Alcohol (190 proof) | 15.0 | 15.0 |
| 6 | Dimethyllsosorbide | 15.0 | 15.0 |
| 7 | Transcutol | 20.0 | 20.0 |
| 8 | Hydroxypropyl cellulose (Klucel | — | 2.0 |
| | FIF PHARMA) | | |
| 9 | Carbopol 980 | 1.60 | — |
| 10 | 2-Amino-2-methyl-1-Propanol (AMP) | 0.5 | — |

### PEG Ointments

The pre-formulation compatibility study of CT327 with PEG was done using PEG 400. PEG 400 is liquid at room temperature. To obtain ointment like consistency a combination of PEG 400 with PEG 3350 was used. The ratio of the two PEGs was varied to determine the ratio of PEG 400 and 3350 to provide the desired stiffness to the ointment.

The formulations listed in Table 11 were evaluated for stiffness. The formulation with the lowest amount of PEG 3350 (30%) had the best spreadability and acceptable consistency. The ratio of PEG 400 to 3350 in formulation 3136-44-1 was selected as the base for PEG ointments. The compositions did not show liquid separation after storage at 50° C. for three weeks.

TABLE 11
FORMULATION COMPOSITIONS OF
PEG BASED PLACEBO OINTMENTS

| | | 3136-44-1 | 3136-44-2 | 3136-44-3 |
|---|---|---|---|---|
| | Ingredients | (% w/w) | (% w/w) | (% w/w) |
| 1 | Polyethylene Glycol | 59.0 | 54.0 | 50.0 |
| | (PEG) 400 | | | |
| 2 | PEG 3350 | 30.0 | 35.0 | 39.0 |
| 3 | Benzyl Alcohol | 5.0 | 5.0 | 5.0 |

During the pre-formulation study it had been observed that the addition of BHT improved the stability of CT327 in PEG. Both BHT and BHA are free-radical scavengers and are listed in the USP/NF. During formulation development, solubility of both BHT and BHA in PEG was assessed visually. BHA was found to have higher solubility in PEG, and was therefore selected for inclusion in further evaluation.

During the pre-formulation study it was also observed that the levels of the amide degradation product in CT327 solutions in PEG increased over time, indicating potential for alkaline hydrolysis of CT327. As noted earlier, the addition of acids was considered, to potentially inhibit this degradation pathway. The solubility of various acids such as lactic acid, citric acid, ascorbic acid and sorbic acid in PEG was investigated. It was found that the following combinations: (1) lactic acid in benzyl alcohol plus PEG 400 and (2) citric acid in ethanol plus PEG 400, formed clear solutions. Thus two PEG prototype formulations were developed, one containing lactic acid and the other containing citric acid.

### Non-Polar Gel

CT327 in a blend of ethanol and non-polar solvents such as caprylic/capric triglycerides, cyclomethicone and isopropyl myristate had shown good physical compatibility. Two non-polar gel placebo prototypes were prepared with minor variations in composition (Table 12). Both gels utilized Cab-o-Sil (fumed silica) as the gelling agent. Formulation 39-1 was a cloudy gel with a smooth feel while 39-2 was a clear gel with smooth feel. These two prototypes were included in R&D stability studies.

TABLE 12
FORMULATION COMPOSITIONS
OF NON-POLAR PLACEBO GELS

| | | 3136-39-1 | 3136-39-2 |
|---|---|---|---|
| | Ingredients | (% w/w) | (% w/w) |
| 1 | CT 327 | — | — |
| 2 | Caprylic/Capric Triglyceride | 43.0 | 43.0 |
| 3 | Isopropyl Myristate | 25.0 | 25.0 |
| 4 | Mineral Oil | — | 15.0 |
| 5 | Cyclomethicone | 15.0 | — |
| 6 | Benzyl Alcohol | 10.0 | 10.0 |
| 7 | Cab-O-Sil M-5 | 7.0 | 7.0 |

### R&D Stability Study

Introduction

Eight formulation prototypes containing either 0.1% or 0.5% CT327 were manufactured and their stability was monitored for up to 6 months at 40° C. and 12 months at 25° C. The compositions of these eight ointments and gels are listed in Tables 13 & 14. They were packaged in both 20 mL and 40 mL clear glass vials and covered with aluminum foil.

Samples filled in 20 mL vials were designated for analytical testing while samples stored in 40 mL vials were designated for physical appearance.

TABLE 13
COMPOSITION OF 0.1% CT327 PROTOTYPES

| | % w/w | | | | | | | |
|---|---|---|---|---|---|---|---|---|
| | | | | | | | | Polar |
| | Petrolatum ointment | | | PEG ointment | | Non-polar gel | | gel |
| | Lot # (3136-) | | | | | | | |
| | 68 (A) | 69 (B) | 70 (C) | 73 | 74 | 71 | 72 | 75 |
| CT327 | 0.1 | 0.1 | 0.1 | 0.1 | 0.1 | 0.1 | 0.1 | 0.1 |
| Propylene | 5.0 | — | 5.0 | 5.0 | — | — | — | 5.0 |
| carbonate | | | | | | | | |
| Benzyl alcohol | — | 5.0 | — | 5.4 | 5.4 | 11.7 | — | 5.0 |
| Alcohol (190 | — | — | — | — | 5.0 | — | 13.6 | 20.4 |
| proof) | | | | | | | | |
| Propylene glycol | 7.8 | 5.8 | — | — | — | — | — | 35.0 |
| BHT | 0.1 | 0.1 | 0.1 | — | — | 0.1 | 0.1 | 0.1 |
| BHA | — | — | — | 0.1 | 0.1 | — | — | — |
| White petrolatum | 83.0 | 75.0 | 80.0 | — | — | — | — | — |
| Cetyl alcohol | 2.0 | — | — | — | — | — | — | — |
| Polyoxyethylene | 2.0 | — | — | — | — | — | — | — |
| (2) Stearyl Ether | | | | | | | | |
| White wax | — | 8.0 | — | — | — | — | — | — |
| Stearyl Alcohol | — | 3.0 | — | — | — | — | — | — |
| Cholesterol | — | 3.0 | — | — | — | — | — | — |
| Para frm | — | — | 3.4 | — | — | — | — | |
| Mineral oil | — | — | 11.4 | — | — | 14.4 | — | — |
| Caprylic/capric | — | — | — | — | — | 44.4 | 44.4 | — |
| triglycericies | | | | | | | | |
| Isopropyl | — | — | — | — | — | 25.0 | 25.0 | — |
| m_yristate | | | | | | | | |
| Cab-O- | — | — | — | — | — | 4.3 | 4.4 | — |
| SiUFurned silica | | | | | | | | |
| Cyclomethicone | — | — | — | — | — | — | 12.4 | — |
| PEG 400 | — | — | — | 30.0 | 30.0 | — | — | — |
| PEG 3350 | — | — | — | 58.4 | 58.4 | — | — | — |
| Lactic acid | — | — | — | 1.0 | — | — | — | — |
| Citric acid | — | — | — | — | 1.0 | — | — | — |
| Transcutol | — | — | — | — | — | — | — | 20.0 |
| Klucel (hydroxyl | — | — | — | — | — | — | — | 2.0 |
| propyl cellulose) | | | | | | | | |

TABLE 14
COMPOSITION OF 0.5% CT327 PROTOTYPES

| | % w/w | | | | | | | |
|---|---|---|---|---|---|---|---|---|
| | | | | | | | | Polar |
| | Petrolatum ointment | | | PEG ointment | | Non-polar gel | | gel |
| | Lot # (3136-) | | | | | | | |
| | 60 (A) | 61 (B) | 62 (C) | 65 | 66 | 63 | 64 | 67 |
| CT327 | 0.5 | 0.5 | 0.5 | 0.5 | 0.5 | 0.5 | 0.5 | 0.5 |
| Propylene | 5.0 | | 5.0 | 5.0 | — | — | — | 5.0 |
| carbonate | | | | | | | | |
| Benzyl alcohol | — | 5.0 | — | 5.0 | 5.0 | 11.7 | — | 5.0 |
| Alcohol (190 | — | — | — | — | 5.0 | — | 13.6 | 20.0 |
| proof) | | | | | | | | |
| Propylene glycol | 7.4 | 5.4 | — | — | — | — | — | 35.0 |
| BHT | 0.1 | 0.1 | 0.1 | | | 0.1 | 0.1 | 0.1 |
| BHA | — | — | — | 0.1 | 0.1 | — | — | — |
| White petrolatum | 83.0 | 75.0 | 80.0 | — | — | — | — | — |
| Cetyl alcohol | 2.0 | — | — | — | — | — | — | — |
| Polyoxyethylene | 2.0 | — | — | — | — | — | — | — |
| (2) Stearyl Ether | | | | | | | | |
| White wax | — | 8.0 | — | — | — | — | — | — |
| Stearyl Alcohol | — | 3.0 | — | — | — | — | — | — |
| Cholesterol | — | 3.0 | — | — | — | | — | — |
| Paraffin | — | — | 3.0 | — | — | — | — | — |
| Mineral oil | — | — | 11.4 | — | — | 14.4 | — | — |
| Caprylic/capric | — | — | — | — | — | 44.0 | 44.0 | — |
| triglyeerides | | | | | | | | |
| Isopropyl | — | — | — | — | — | 25.0 | 25.0 | — |
| myristate | | | | | | | | |
| Cab-0-Sil/Fumed | — | — | — | — | — | 4.3 | 4.4 | — |
| silica | | | | | | | | |
| Cyclomethicone | — | — | — | — | — | — | 12.4 | — |
| PEG 400 | — | — | — | 30.0 | 30.0 | — | — | — |
| PEG 3350 | — | — | — | 58.4 | 58.4 | — | — | — |
| Lactic acid | — | — | — | 1.0 | — | — | — | — |
| Citric acid | — | — | — | — | 1.0 | — | — | — |
| Transcutol | — | — | — | — | — | — | — | 20.0 |
| Klucel (Hydroxy | — | — | — | — | — | — | — | 2.0 |
| propylcellulose) | | | | | | | | |

Testing

At 1 month, all samples (stored at both 25° C. and 40° C.) were visually evaluated for appearance but only samples stored at 40° C. were analytically tested. At 2, 3, and 6 months samples stored at both 25° C. and 40° C. were tested for physical appearance and chemical stability. At 9 and 12 months only samples stored at 25° C. were tested.

Degradation products were determined by comparing chromatograms of each sample at different stability time points. For example, degradation product results at 1 month were determined by comparing 1 month chromatograms with that of time zero. Any peaks appearing at a later timepoint that did not have a time zero counterpart was counted as a degradation product, and its percent area was reported

There is no specification for degradation products of CT327 anhydrous product but there are impurity specifications for CT327. This specification (listed in Table 15 below) for identified impurities/degradation products was included in CT327 CMC. This specification will be used only as guideline. The anhydrous CT327 topical formulation prototypes could have higher amount of degradation products and other degradation product s that have not been observed in the drug CT327.

TABLE 15
QUALIFIED IDENTIFIED IMPURITIES

| Impurities (RRT % area) | Control Limit |
|---|---|
| RRT 0.4.4-0.46 | ≤1.5% |
| RRT 0.66-0.70 | ≤0.1% |
| RRT 0.87-0.91 | ≤0.2% |
| RRT 1.89-1.92 | ≤0.2% |

Total sum of impurities: Not more than 2.0% in total.

Results

### Petrolatum Based Ointments

Batch numbers (0.5%): 3136-60A, 61B, 62C

Batch numbers (0.1%): 3136-68A, 69B, 70C

One Month

All the petrolatum based ointments were physically stable at both 25° C. and 40° C. The ointment prototypes had a smooth white to off-white appearance (Table 16).

TABLE 16
APPEARANCE OF R&D STABILITY SAMPLES

| Lot # | Time | 1 month | | 2 months | | 3 months | |
|---|---|---|---|---|---|---|---|
| (3136-) | Zero | 25 C. | 40 C. | 25 C. | 40 C. | 25 C. | 40 C. |
| 60 (A) | OWC | OWC | OWC | OWC | OWC | OWC | OWC |
| 61 (B) | OWC | OWC | OWC | OWC | OWC | OWC | OWC |
| 62 (C) | OWC | OWC | OWC | OWC | OWC | NT | NT |
| 63 | IIG & LS | HG & LS | HG & LS | HG & LS | HG & LS | NT | NT |
| 64 | CG & LS | CO & LS | CO & LS | CO & LS | CO & LS | NT | NT |
| 65 | WC | WC & | WC | OWC | OWC | OWC | OWC & |
| | | LS | | | | | LS |
| 66 | WC | WC | WC | OWC | OWC | NT | NT |
| 67 | CO | CO | CO | CO | CO | CU | CG |
| 68 (A) | OWC | OWC | OWC | OWC | OWC | OWC | OWC |
| 69 (B) | OWC | OWC | OWC | OWC | OWC | OWC | OWC |
| 70 (C) | OWC | OWC | OWC | OWC | OWC | NT | NT |
| 71 | CG | 110 | 1-10 | HG & LS | HO & LS | NT | NT |
| 72 | CU | HG | HG | CG & LS | CG & LS | NT | NT |
| 73 | WC | WC | WC | OWC | OWC | OWC | OWC & |
| | | | | | | | LS |
| 74 | WC | WC | WC | OWC | OWC | NT | NT |
| 75 | CG | CO | CG | CG | CG | CO | CG |

OWC—off white cream/ointment
WC—white cream/ointment
HO—hazy gel
CG—clear, transparent gel
LS—liquid separation observed (typically small amount of liquid floating at the top)
NT—not tested

Petrolatum ointment prototypes A and B, 0.5% and 0.1% respectively (60, 61, 68 and 69) showed some variability in assay values but no consistent trend at either concentration—0.1 and 0.5% (Table 17). Some degradant peaks were observed both at 25° C. and 40° C. (Table 18). Prototype C at both concentrations, 0.5% and 0.1%, showed a decrease in the assay values.

TABLE 17
EFFECT OF STORAGE TIME AND TEMPERATURE ON ASSAY
VALUES
Assay (% label claim)

| Lot # | Time | 1 month | | 2 months | | 3 months | | 6 months | |
|---|---|---|---|---|---|---|---|---|---|
| (3136-) | Zero | 25° C. | 40° C. | 25° C. | 40° C. | 25° C. | 40° C. | 25° C. | 40° C. |

0.5% prototypes

| | | | | | | | | | |
|---|---|---|---|---|---|---|---|---|---|
| 60 (A) | 107.4 | NT | 104.6 | 101.2 | 105.5 | 102.2 | 103.3 | 104.5 | 121.4 |
| 61 (B) | 92.3 | NT | 93.2 | 93.3 | 91.3 | 95.6 | 93.2 | 101.7 | 101.5 |
| 62 (C) | 103.5 | NT | 96.3 | 85.0 | 87.0 | NT | NT | NT | NT |
| 63 | 102.5 | NT | 92.8 | NT | NT | NT | NT | NT | NT |
| 64 | 105.8 | NT | 112.4 | NT | NT | NT | NT | NT | NT |
| 65 | 102.1 | NT | 100.9 | 101.5 | 96.3 | 99.9 | 94.4 | 100.8 | 98.1 |
| 66 | 102.6 | NT | 97.2 | 102.0 | 92.6 | NT | NT | NT | NT |
| 67 | 103.6 | NT | 104.0 | 110.1 | 121.9 | 103.5 | 104.3 | 106.0 | 121.6 |

0.1% prototypes

| | | | | | | | | | |
|---|---|---|---|---|---|---|---|---|---|
| 68 (A) | 104.4 | NT | 111.5 | 109.2 | 109.1 | 110.8 | 104.9 | 109.0 | 115.4 |
| 69 (B) | 99.1 | NT | 109.4 | 101.0 | 102.1 | 102.0 | 101.8 | 110.2 | 111.2 |
| 70 (C) | 108.2 | NT | 105.4 | 87.6 | 100.8 | NT | NT | NT | NT |
| 71 | 102.4 | NT | 97.0 | NT | NT | NT | NT | NT | NT |
| 72 | 106.4 | NT | 115.1 | NT | NT | NT | NT | NT | NT |
| 73 | 102.8 | NT | 99.4 | 102.6 | 96.5 | 103.0 | 95.2 | 99.1 | 84.3 |
| 74 | 105.6 | NT | 97.5 | 104.2 | 89.3 | NT | NT | NT | NT |
| 75 | 103.2 | NT | 106.3 | 109.7 | 104.0 | 110.8 | 117.4 | 114.7 | 105.0 |

TABLE 18
MONTH 1 DEGRADATION PRODUCTS

| | | RRT of | | |
|---|---|---|---|---|
| Lot # | Replicate | degradation | | |
| (3136-) | Injections | product | % Area | Total |

0.5% prototypes

| | | | | |
|---|---|---|---|---|
| 60 (A) | lnj 1 | 0.44 | 0.28 | 0.28 |
| | Inj 2 | 0.44 | 0.20 | 0.20 |
| 61 (B) | lnj 1 | 1.80 | 0.11 | 0.11 |
| | Inj 2 | 1.61 | 0.12 | 0.12 |
| 62 (C) | lnj 1 | ND | ND | ND |
| | Inj 2 | ND | ND | ND |
| 63 | In] 1 | 0.47 | 0.18 | 5.56 |
| | | 0.69 | 3.82 | |
| | | 2.43 | 1.56 | |
| | Inj 2 | 0.46 | 0.113 | 5.70 |
| | | 0.69 | 3.868 | |
| | | 2.44 | 1.716 | |
| 64 | Inj I | 0.45 | 0.70 | 0.70 |
| | Inj 2 | 0.44 | 0.59 | 039 |
| 65 | Inj 1 | 0.46 | 0.47 | 0.47 |
| | Inj 2 | 0.45 | 0.65 | 0.65 |
| 65 | Inj I | 0.41 | 0.179 | 0.24 |
| | | 1.70 | 0.061 | |
| | Inj 2 | 0.41 | 0.151 | 0.25 |
| | | 1.69 | 0.097 | |
| 67 | Intl | 0.45 | 0.63 | 0.63 |
| | Inj 2 | 0.45 | 0.64 | 0.64 |

0.1% prototype

| | | | | |
|---|---|---|---|---|
| 68 (A) | Inj 1 | 0.44 | 0.29 | 0.29 |
| | Inj 2 | 0.45 | 0.36 | 0.36 |
| 69 (B) | Inj 1 | 0.44 | 0.18 | 1.10 |
| | | 218 | 0.71 | |
| | | 2.27 | 0.22 | |
| | Inj 2 | 0.44 | 0.33 | 1.28 |
| | | 2.18 | 0.75 | |
| | | 2.27 | 0.20 | |
| 70 (C) | Inj 1 | ND | ND | ND |
| | Inj 2 | ND | ND | ND |
| 71 | Inj 1 | 0.45 | 0.292 | 0.98 |
| | | 0.67 | 0.685 | |
| | lnj 2 | 0.44 | 0.194 | 1.94 |
| | | 0.69 | 1.748 | |
| 72 | Inj 1 | 0.45 | 1.27 | 1.27 |
| | Inj 2 | 0.44 | 1.33 | 1.33 |
| 73 | Inj 1 | 0.45 | 0.62 | 0.62 |
| | Inj 2 | 0.45 | 0.57 | 0.57 |
| 74 | inj 1 | 0.41 | 0.73 | 0.73 |
| | Inj 2 | 0.41 | 0.63 | 0.63 |
| 75 | lnj 1 | 0.44 | 0.77 | 0.77 |
| | Inj 2 | 0.45 | 0.61 | 0.61 |

Prototype A has degradant peak RRT (0.44) which falls in the range of identified impurity for drug CT327 (0.44-0.46). The peak area is <0.5%. The total sum of degradation product is less than 0.5% for both prototypes A and B. Degradant peaks were not detected in prototype C.

Two Months

All petrolatum-based ointments of both strengths 0.5% and 0.1% at 25° C. and 40° C. were physically stable. No change in the appearance was noted.

Prototypes A and B at both concentrations 0.5% and 0.1% (60, 61, 68 and 69) showed no significant loss in assay values after 2 months at 40° C. (Table 19). Minimal degradation products for these prototypes were observed (Table 20).

TABLE 19
EFFECT OF STORAGE TIME AND TEMPERATURE ON ASSAY
VALUES

| | Assay (% label claim) | | | | | | | | |
|---|---|---|---|---|---|---|---|---|---|
| | | Time | | | | | | | |
| Lot # | | 1 month | | 2 months | | 3 months | | 6 months | |
| (3136-) | Zero | 25° C. | 40° C. | 25° C. | 40° C. | 25° C. | 40° C. | 25° C. | 40° C. |

0.5% prototypes

| | | | | | | | | | |
|---|---|---|---|---|---|---|---|---|---|
| 60 (A) | 107.4 | NT | 104.6 | 101.2 | 105.5 | 102.2 | 103.3 | 104.5 | 121.4 |
| 61 (B) | 92.3 | NT | 93.2 | 93.3 | 91.3 | 95.6 | 93.2 | 101.7 | 101.5 |
| 62 (C) | 103.5 | NT | 96.3 | 85.0 | 87.0 | NT | NT | NT | NT |
| 63 | 102.5 | NT | 92.8 | NT | NT | NT | NT | NT | NT |
| 64 | 105.8 | NT | 112.4 | NT | NT | NT | NT | NT | NT |
| 65 | 102.1 | NT | 100.9 | 101.5 | 96.3 | 99.9 | 94.4 | 100.8 | 98.1 |
| 66 | 102.6 | NT | 97.2 | 102.0 | 92.6 | NT | NT | NT | NT |
| 67 | 103.6 | NT | 104.0 | 110.1 | 121.9 | 103.5 | 104.3 | 106.0 | 121.6 |

0.1% prototypes

| | | | | | | | | | |
|---|---|---|---|---|---|---|---|---|---|
| 68 (A) | 104.4 | NT | 111.5 | 109.2 | 109A | 110.8 | 104.9 | 109.0 | 115.4 |
| 69 (B) | 99.1 | NT | 109.4 | 101.0 | 102.1 | 102.0 | 101.8 | 110.2 | 111.2 |
| 70 (C) | 108.2 | NT | 105.4 | 87.6 | 100.8 | NT | NT | NT | NT |
| 71 | 102.4 | NT | 97.0 | NT | NT | NT | NT | NT | NT |
| 72 | 106.4 | NT | 115.1 | NT | NT | NT | NT | NT | NT |
| 73 | 102.8 | NT | 99.4 | 102.6 | 96.5 | 103.0 | 95.2 | 99.1 | 84.3 |
| 74 | 105.6 | NT | 97.5 | 104.2 | 89.3 | NT | NT | 1 T | NT |
| 75 | 103.2 | NT | 106.3 | 109.7 | 104.0 | 110.8 | 117.4 | 114.7 | 105.0 |

TABLE 20
MONTH 2 DEGRADATION PRODUCTS

| | | 2 months 25° C. | | | 2 months 40° C. | | |
|---|---|---|---|---|---|---|---|
| | | RRT of | | | RRT of | | |
| | | degra- | | | degra- | | |
| Lot # | Replicate | dation | % | | dation | % | |
| (3136-) | Injection | product | Area | Total | product | Area | Total |

0.5% prototype

| | | | | | | | |
|---|---|---|---|---|---|---|---|
| 60 (A) | Inj 1 | ND | ND | ND | 0.44 | 0.57 | 0.57 |
| | Inj 2 | ND | ND | ND | 0.44 | 0.48 | 0.48 |
| 61 (B) | Inj 1 | ND | ND | ND | ND | ND | ND |
| | Inj 2 | ND | ND | ND | ND | ND | ND |
| 62 (C) | Inj 1 | ND | ND | ND | ND_{—} | ND | NE) |
| | Inj 2 | ND | ND | ND | ND | ND | ND |
| 65 | Inj 1 | ND | ND | ND | 0.44 | 0.78 | 0.78 |
| | Inj 2 | ND | ND | ND | 0.44 | 0.58 | 0.58 |
| 66 | Inj 1 | ND | ND | ND | 0.40 | 1.346 | 3.45 |
| | | | | | 1.71 | 2.106 | |
| | Inj 2 | ND | ND | ND | 0.40 | 1.115 | 3.30 |
| | | | | | 1.69 | 2.187 | |
| 67 | Inj 1 | 0.44 | 0.29 | 0.29 | 0.44 | 0.94 | 0.94 |
| | Inj 2 | 0.44 | 0.2S | 0.28 | 0.44 | 0.97 | 0.97 |

0.1% prototype

| | | | | | | | |
|---|---|---|---|---|---|---|---|
| 68 (A) | Inj I | ND | ND | ND | 0.44 | 0.24 | 0.24 |
| | Inj 2 | ND | ND | ND | 0.44 | 0.46 | 0.46 |
| 69 (B) | Inj 1 | ND | ND | ND | ND | ND | ND |
| | Inj 2 | ND | ND | ND | ND | ND | ND |
| 70 (C) | Inj 1 | ND | ND | ND | ND | ND | ND |
| | Inj 2 | ND | ND | ND | ND | ND | ND |
| 73 | Inj 1 | ND | ND | ND | 0.43 | 0.79 | 0.79 |
| | Inj 2 | ND | ND | ND | 0.44 | 0.72 | 0.72 |
| 74 | Inj 1 | ND | ND | ND | 0.40 | 0.63 | 0.63 |
| | Inj 2 | ND | ND | ND | 0.40 | 1.26 | 1.26 |
| 75 | Inj 1 | 0.45 | 0.40 | 0.40 | 0.44 | 0.82 | 0.82 |
| | Inj 2 | 0.45 | 0.32 | 032 | 0.44 | 0.71 | 0.71 |

Prototypes C, samples 62 and 70, showed a decreasing trend in CT327 assay values. This was especially true of sample 62 (0.5% CT327) (Table 19). Hence petrolatum prototype C was dropped from further evaluation.

Three and Six Months

After 6 months of storage at 25° C. and 40° C. prototypes A & B (formula #s 60 and 61, 0.5%) and (68 and 69, 0.1%) showed no significant drop in assay values (Table 19).

Prototype A at concentrations, 0.5 and 0.1% (samples 60 and 68), show increase in degradant peak area over time (Tables 21 & 22). Degradant peak RRT, for prototype A; 60 and 68 (0.5% and 0.1% respectively), falls in the range of identified impurity for drug CT327 (0.44-0.46). Data show that the degradation products increase over time. Sum of the degradation products increases from <0.5% at 1 month to >2% at 6 months. The identified degradant peak (RRT 0.44-0.46) also exceeds 1.5% after 6 months at 40° C. Hence prototype A was no longer considered a lead candidate for CT327 topical formulation.

In petrolatum prototype B no apparent trend in degradation products (Tables 21 & 22) is observed. At some stability intervals degradant peak area is: 1.2% and in other intervals it is <0.5% or undetected. Since no clear trend in degradant levels was observed, petrolatum prototype B was selected for further consideration.

TABLE 21
MONTH 3 DEGRADATION PRODUCTS

| | | 3 months 25° C. | | | 3 months 40° C. | | |
|---|---|---|---|---|---|---|---|
| | | RRT of | | | RRT of | | |
| | | degra- | | | degra- | | |
| Lot # | Replicate | dation | % | | dation | | |
| (3136-) | Injection | product | Area | Total | product | % Area | Total |

0.5% prototypes

| | | | | | | | |
|---|---|---|---|---|---|---|---|
| 60 (A) | Inj 1 | ND | NA | ND | 0.45 | 1.27 | 1.27 |
| | Inj 2 | ND | NA | ND | 0.45 | 1.34 | 1.34 |
| 61 (B) | Inj 1 | 0.58 | 0.41 | 0.41 | 0.59 | 0.179 | 0.24 |
| | | | | | 0.60 | 0.061 | |
| | Inj 2 | 0.58 | 0.31 | 0.31 | 0.59 | 0.151 | 0.25 |
| | | | | | 0.60 | 0.097 | |
| 65 | Inj 1 | 0.45 | 0.32 | 0.32 | 0.45 | 1.17 | 1.17 |
| | Inj 2 | 0.45 | 0.40 | 0.40 | 0.45 | 1.29 | 1.29 |
| 67 | Inj 1 | 0.45 | 0.346 | 0.53 | 0.45 | 1.336 | 1.52 |
| | | 0.57 | 0.183 | | 0.57 | 0.186 | |
| | Inj 2 | 0.45 | 0.307 | 0.44 | 0.45 | 1.228 | 1.42 |
| | | 0.57 | 0.135 | | 0.57 | 0.190 | |

0.1% prototypes

| | | | | | | | |
|---|---|---|---|---|---|---|---|
| 68 (A) | Inj 1 | ND | NA | ND | 0.44 | 0.60 | 0.60 |
| | Inj 2 | ND | NA | ND | 0.45 | 0.79 | 0.79 |
| 69 (B) | Inj 1 | 0.58 | 0.385 | 0.86 | 0.46 | 0.310 | 1.29 |
| | | 0.59 | 0.478 | | 0.58 | 0.382 | |
| | | | | | 0.60 | 0.595 | |
| | Inj 2 | 0.58 | 0.336 | 0.80 | 0.4 | 0.244 | 0.98 |
| | | 0.60 | 0.469 | | 0.57 | 0.275 | |
| | | | | | 0.59 | 0.465 | |
| 73 | Inj 1 | ND | NA | ND | 0.44 | 0.98 | 0.98 |
| | Inj 2 | ND | NA | ND | 0.44 | 0.83 | 0.83 |
| 75 | Inj 1 | 0.44 | 0.370 | 0.99 | 0.44 | 1.435 | 1.99 |
| | | 0.58 | 0.623 | | 0.58 | 0.560 | |
| | Inj 2 | 0.44 | 0.494 | 1.11 | 0.44 | 1.315 | 1.87 |
| | | 0.58 | 0.616 | | 0.58 | 0.557 | |

TABLE 22
MONTH 6 DEGRADATION PRODUCTS

| | | 6 months 25° C. | | | 6 months 40° C. | | |
|---|---|---|---|---|---|---|---|
| | | RRT of | | | RRT of | | |
| | | degra- | | | degra- | | |
| Lot # | Replicate | dation | % | | dation | | |
| (3136-) | Injection | product | Area | Total | product | % Area | Total |

0.5% prototypes

| | | | | | | | |
|---|---|---|---|---|---|---|---|
| 60 (A) | Inj 1 | ND | NA | ND | 0.45 | 2.03 | 2.03 |
| | Inj 2 | ND | NA | ND | 0.45 | 1.86 | 1.86 |
| 61 (B) | Inj 1 | ND | NA | ND | 0.46 | 0.34 | 0.34 |
| | Inj 2 | ND | NA | ND | 0.45 | 0.31 | 0.31 |
| 65 | Inj 1 | 0.45 | 0.38 | 0.38 | 0.45 | 1.49 | 1.49 |
| | Inj 2 | 0.45 | 0.42 | 0.42 | 0.44 | 1.77 | 1.77 |
| 67 | Inj 1 | 0.46 | 0.40 | 0.40 | 0.45 | 1.336 | 1.68 |
| | Inj 2 | 0.45 | 0.29 | 0.29 | 0.45 | 1.228 | 1.69 |

0.1% prototypes

| | | | | | | | |
|---|---|---|---|---|---|---|---|
| 68 (A) | Inj 1 | ND | NA | ND | 0.45 | 1.82 | 1.82 |
| | Inj 2 | ND | NA | ND | 0.61 | 0.276 | 2.46 |
| | | | | | 0.63 | 0.445 | |
| | | | | | 1.72 | 0.572 | |
| | | | | | 1.94 | 0.684 | |
| | | | | | 3.39 | 0.350 | |
| | | | | | 3.70 | 0.138 | |
| 69 (B) | Inj 1 | 3.66 | 0.14 | 0.14 | 3.69 | 0.13 | 0.13 |
| | Inj 2 | 2.45 | 0.189 | 0.34 | 3.67 | 0.10 | 0.10 |
| | | 3.69 | 0.146 | | | | |
| 73 | Inj 1 | ND | NA | ND | 0.38 | 0.145 | 1.88 |
| | | | | | 0.45 | L51 6 | |
| | | | | | 3.76 | 0.221 | |
| | Inj 2 | ND | NA | ND | 0.38 | 0.176 | 2.54 |
| | | | | | 0.44 | 1.529 | |
| | | | | | 3.75 | 0.834 | |
| 75 | Inj 1 | 0.45 | 0.30 | 0.30 | 0.45 | 1.58 | 1.58 |
| | Inj 2 | 0.45 | 0.38 | 0.38 | 0.45 | 1.49 | 1.49 |

Nine and Twelve Months at 25° C.—Prototype B

No obvious drop in assay values was observed at either concentration-0.5% or 0.1%, samples 3136-61 and 69 respectively (Table 23). Similarly, no apparent trend in degradation peak areas can be detected. While at some stability time points minimal degradation peak areas are observed at other time points they are undetected (Table 24).

TABLE 23
ASSAY VALUES OF THE PROMISING
PROTOTYPES AT 9 AND 12 MONTHS

Assay % Label claim

| | | |
|---|---|---|
| | 9 mo. | 12 mo. |
| Lot # | 25° C. | 25° C. |

0.5% prototypes

| | | |
|---|---|---|
| 3136-61 (B) | 92.1 | 96.5 |
| 3136-67 | 105.8 | 111.9 |

0.1% prototypes

| | | |
|---|---|---|
| 3136-61 (B) | 102.6 | 102.6 |
| 3136-75 | 110.0 | 112.1 |

TABLE 24
MONTH 9 AND 12 DEGRADATION PRODUCTS

| | | 9 months 5° C. | | | 12 months 25° C. | | |
|---|---|---|---|---|---|---|---|
| | | RRT of | | | RRT of | | |
| | | degra- | | | degra- | | |
| Lot # | Replicate | dation | % | | dation | % | |
| (3136-) | Injection | product | Area | Total | product | Area | Total |

0.5% prototype

| | | | | | | | |
|---|---|---|---|---|---|---|---|
| 61(B) | Inj 1 | NA | ND | ND | 0.45 | 0.195 | 0.19 |
| | In 2 | NA | ND | ND | 0.45 | 0.161 | 0.16 |
| 67 | Inj 1 | 0.45 | 1.302 | 1.30 | 0.44 | 1.637 | 1.64 |
| | Inj 2 | 0.45 | 1.281 | 1.28 | 0.44 | 1.644 | 1.64 |

0.1% prototype

| | | | | | | | |
|---|---|---|---|---|---|---|---|
| 69(B) | Inj 1 | NA | ND | ND | 0.43 | 0.187 | 0.19 |
| | Inj 2 | NA | ND | ND | 0.43 | 0.232 | 0.23 |
| 75 | Inj 1 | 0.45 | 0.887 | 0.89 | 0.44 | 1.859 | 1.86 |
| | Inj 2 | 0.45 | 0.860 | 0.86 | 0.44 | 1.869 | 1.87 |

### Polar Gels

Batch numbers (0.5%): 3136-67

Batch numbers (0.1%): 3136-75

One Month

After 1 month at 25° C. and 40° C., the polar gels at both strengths, 0.5% and 0.1%, were physically stable (Table 16).

While degradants were observed in the samples stored at 40° C., their peak area in both samples 67 and 75 is less than 1.0%.

Two Months

After 2 months at 40° C., the assay results for prototype 67 showed much higher values than expected (Table 19). One hypothesis was the possible precipitation of the drug substance, either with aging of the product or evaporation of the volatile solvents. Observation of the sample through the vial wall using a magnifying glass, indicated the presence of lint like particles. Since this formulation is a cellulose-based gel, there might be lint-like particles that are carried in with hydroxypropyl cellulose or from any undissolved grains of HPC. The particles could also have been precipitated drug. This could not be resolved at that time, and it was decided to wait for the 3-month data to become available.

Three & Six Months

Prototypes 67 and 75 did not show significant drop in the assay after 6 months at 40° C. (Table 19). There is variability in the assay results, which might be because the analytical method is not tailored for this prototype.

On comparing the degradation product results for samples stored at 40° C., at various time points: 1, 2, 3, and 6 months an increase in degradation products over time is observed (Tables 18, 20, 21 & 22). For instance, total sum of degradation products for prototype 67 at 40° C. increases from 0.64 to 0.97 to 1.52 to 1.69 at 1 mo, 2 mo, 3 mo., and 6 mo. respectively. Even at 25° C. a slight increase in % peak area of degradation products over time is evident. However, for both concentrations 67 and 75 the total % peak area of degradation products did not exceed 2.0% after 6 months at 40° C. Hence polar gel prototype was selected as one of the lead candidates for CT327 topical formulation.

Nine & Twelve Months

Prototypes 67 and 75 did not show significant drop in the drug level after 12 months at 25° C. (Table 23). There is variability in the assay results. However, an increase in degradation product total peak area over 12 months at 25C is evident at both concentrations: 0.5% and 0.1% (Table 24). Sample 3136-67 (0.5%) shows an increase in total degradation product peak area from 0.3% at 2 months to 1.6% at 12 months, while 3136-75 (0.1%) also shows an increase from 0.4% at 2 months to 1.9% at 12 months (Tables 20 & 24).

### PEG Based Ointments

Batch numbers (0.5%): 3136-65, 66

Batch numbers (0.1%): 3136-73, 74

One Month

After 1 month of storage at 25° C. and 40° C. all the PEG ointments were physically stable (Table 16).

PEG prototypes 65, 73, which have identical formulation composition except for differences in drug concentrations 0.5% and 0.1%, showed <4% drop in assay values after storage at 40° C. for 1 month, while PEG prototypes 66 and 74 showed >5% decrease in assay values (Table 17). Degradant peaks were observed for both prototypes (Table 18). RRT of the degradant peak, for prototypes 65 and 73, falls in the range of identified impurity for drug CT327 (0.44-0.46). The peak area of the degradant peak for both samples 65 and 73 are <1.0%. The total sum of degradation products is less than 1.0% for all PEG based ointments 65, 73, 66, and 74.

Two Months

After 2 months of storage at 25° C. and 40° C. all the PEG ointments were physically stable (Table 16).

PEG based ointment prototypes 65 and 73 showed better stability than prototypes 66 and 74, both in terms of loss in potency and increase in degradation product (Table 14 and Table 20). Hence PEG prototypes 66 and 74 were eliminated from further testing.

Three & Six Months

After 6 months of storage at 40° C. it was observed that prototypes 65 and 73 (Table 19) showed a trend of significant loss in potency, especially at the lower concentration (0.1%), which showed >15% loss in potency. Hence PEG based ointments were not considered for further evaluation.

### Non Polar Gels

Batch numbers (0.5%): 3136-63, 64

Batch numbers (0.1%): 3136-71, 72

One Month

Non-polar gels 0.5% (63 and 64) showed liquid separation and appeared hazy at both 25° C. and 40° C. (Table 16). Non-polar gels 0.1% (71 and 72) appeared hazy but did not show liquid separation. Prototypes 63 and 71 showed >5% drop in potency (Table 19) and degradant peaks with total sum of degradation products >5% and 2: 2% respectively (Table 18). Given their lack of physical and chemical stability, non-polar gels were eliminated from further consideration and were not tested at the next stability time points.

Conclusions

Based on the 12-month R&D stability study, suitability for the intended indication and evaluation of product aesthetics, two candidates were selected for consideration (a) petrolatum prototype B (0.5%: 3136-61B and 0.1%: 3136-69B); and (b) polar gel prototype (0.5%: 3136-67 and 0.1%: 3136-75).

### Example 2: In Vitro Percutaneous Absorption of CT327 from Prototype Formulations Using Human Tissue

Drug product efficacy is determined by controlled clinical trials. Bioavailability and potency of topically applied active pharmaceutical ingredients are key components in efficacious drug products. Potential cutaneous (local) and/or systemic bioavailability can be assessed using in vitro percutaneous absorption testing. Data generated using in vitro skin permeation models can support formulation selection during pharmaceutical development programs.

Topical formulations containing CT327 are under development for the treatment of psoriasis/atopic dermatitis. The purpose of this study was to characterize in vitro percutaneous absorption of CT327 from topical formulations following application to excised human skin from elective surgery.

This study was conducted using procedures adapted from the FDA and AAPS Report of the Workshop on Principles and Practices of In Vitro Percutaneous Penetration Studies: Relevance to Bioavailability and Bioequivalence (Skelly et al., 1987). Human tissue from a single donor was dosed with 5 mg/cm² of formulation.

Data from this preliminary in vitro skin permeation experiment indicated that prototypes CT327CP4 (D) exhibited the highest efficiency of delivery for CT327, while CT327OP3 (C) yielded the highest tissue deposition of CT327.

In terms of combined CT327 deposition into the epidermis and dermis, a dose dependent trend with the ointment prototypes is seen. As the concentration of CT327 increases from 0.05% to 5.00%, the combined deposition into the tissues also increases from 584 ng/cm² to 43,984 ng/cm².

In addition to the delivery potential of the prototypes, final prototype selection is expected to factor in other evaluation parameters such as physical and chemical stability of the formulations, disease state and availability of supporting toxicological data.

### Introduction

Drug product efficacy is determined by controlled clinical trials. Bioavailability and potency of topically applied active pharmaceutical ingredients are key components in efficacious drug products. Potential cutaneous (local) and/or systemic bioavailability can be assessed using in vitro percutaneous absorption testing. Data generated using in vitro skin permeation models can support formulation selection during pharmaceutical development programs.

Topical formulations containing CT327 are under development for the treatment of Psoriasis/Atopic Dermatitis. The purpose of this study was to characterize in vitro percutaneous absorption of CT327 from prototype formulations following application to excised human skin from elective surgery.

In vitro skin permeation methodology was utilized to assess the effect of formulation modification on the delivery of CT327 from prototype compositions.

In vitro skin permeation data is intended to facilitate identification of formulation candidates with the highest potential of success, in terms of developing a topical formulation with appropriate delivery characteristics. This approach may also provide the highest potential for correlating in vitro permeation data with clinical efficacy, in diseases where the site of action is located in the viable tissues of the skin.

### Methods

This in vitro percutaneous absorption study was conducted using procedures adapted from the FDA and AAPS Report of the Workshop on Principles and Practices of In Vitro Percutaneous Penetration Studies: Relevance to Bioavailability and Bioequivalence (Skelly et al., 1987). The compositions of the CT327 formulations evaluated in this study are summarized in Table 25. All laboratory activities with the CT327 were performed under yellow lights.

TABLE 25
FORMULATION COMPOSITIONS

| | CT327 ointment | | | CT327 cream |
|---|---|---|---|---|
| Ingredients | % w/w | | | |
| T327 | 0.05 | 0.5 | 5 | 0.1 |
| BHT | 0.1 | 0.1 | 0.1 | — |
| Benzyl Alcohol | 5 | 5 | 10.5 | — |
| Propylene Glycol | 5.85 | 5.4 | 5.4 | — |
| White Petrolatum | 75 | 75 | 65 | — |
| White Wax | 8 | 8 | 8 | — |
| Stearyl Alcohol | 3 | 3 | 3 | — |
| Cholesterol | | 3 | 3 | — |
| Cetostearyl alcohol | — | — | — | 14.4 |
| Cetomacrogol | — | — | — | 3.6 |
| White soft paraffin | — | — | — | 9 |
| Liquid paraffin | — | — | — | 3 |
| pH 4.0 citrate | — | — | — | 58.9 |
| Glycerol | — | — | — | 10 |
| Belay] alcohol | — | — | — | 1 |
| Total | 100 | 100 | 100 | 100 |

The clinically relevant dose of 5 mg/cm² of formulation was applied to dermatomed human abdominal tissue from a single donor obtained following elective surgery. The thickness of the tissue ranged from 0.022-0.033 inches (0.559-0.838 mm) with a mean+/−standard deviation in thickness of 0.028+/−0.003 inches (0.708+/−0.084 mm) and a coefficient of variation of 11.9%. After dosing, the tissue was left un-occluded and undisturbed for the 24-hour exposure period.

Percutaneous absorption was evaluated using this human abdominal tissue from a single donor mounted in Bronaugh flow-through diffusion cells (54 cells in total, 6 replicates each condition). The cells were maintained at a constant temperature of 32° C. by use of recirculating water baths. These cells have a nominal diffusion area of 0.64 cm². Following the 24-hour duration exposure, the formulation residing on the tissue surface was removed by tapestripping with CuDerm D-Square stripping discs. These have been retained for potential future analysis. The epidermis was separated from the dermis by blunt dissection. The epidermis and dermis samples were labeled and frozen prior to subsequent analysis of CT327 content by LC/Fluorescence.

The tissue sample limits of quantification achieved by the R&D LC/Fluorescence analytical method are presented in Table 26.

TABLE 26
LIMITS OF QUANTIFICATION OBTAINED USING THE
R&D ANALYTICAL METHOD FOR TISSUE SAMPLES

| | LOQ (ng/mL) |
|---|---|
| CT327 | 4.408 |

LC/Fluorescence was selected as the detection technique for this study. LC/MS was investigated, but was less sensitive than the LC/fluorescence technique.

The method details are listed below:

Column: Waters)(Bridge C18, 3.5 μm, 4.6×75 mm

Flow Rate: 0.5 mL/min

MPA: 5% MeOH, 95% H2O, 1% glacial acetic acid

MPB: 100% MeOH, 1% glacial acetic acid

Gradient: Isocratic, 30% A, 70% B

Injection Volume: 5 μL

Column Temperature: 40° C.

Autosampler Temperature: 20° C.

Runtime: 5 minutes

Ex: 335 nm

Em: 400 nm

Tissue permeation and deposition results were statistically evaluated using unpaired student's t-tests (significant differences between formulations were defined by a p-value of <0.05, at the 95% confidence interval). Significant differences between formulations were defined by a p value of <0.05. Outlier tests were performed using Grubbs Test. Outliers in the tissue data whether detected in the epidermis data or in the dermis data were removed from the analysis of both tissue types.

### Results

The results of dermal and epidermal deposition are summarized in Table 27.

TABLE 27
CUMULATIVE TISSUE LEVELS OF CT327 FOLLOWING
24 HOURS OF TOPICAL EXPOSURE

| | | Epidermis | | Dermis | | Epidermis + Dermis | |
|---|---|---|---|---|---|---|---|
| | | | Calculated | | Calculated | | Calculated |
| | | | ng/cm² API | | ng/cm² APT | | ng/cm² API |
| | | | for | | for | | for |
| | | | Formulation | | Formulation | | Formulation |
| | | % Dose | Dose of | % Dose | Dose of | % Dose | Dose of |
| Formulation ID | | Applied | 5 mg/cm² | Applied | 5 mg/cm² | Applied | 5 mg/cm² |
| A) | Mean | 17.5 | 437 | 5.87 | 147 | 23.4 | 584 |
| CT3270P1 | SD | 6.72 | 168 | 3.75 | 94 | 9.39 | 235 |
| 0.05% | % CV | 38 | 38 | 64 | 64 | 40 | 40 |
| B) | Mean | 13.0 | 3244 | 1.46 | 365 | 14.4 | 3609 |
| CT3270P2 | SD | 2.50 | 624 | 0.36 | 90 | 2.45 | 614 |
| 0.5% | % CV | 19 | 19 | 25 | 2.5 | 17 | 17 |
| C) | Mean | 14.9 | 37354 | 2.65 | 6630 | 17.6 | 43984 |
| CT3270P3 | SD | 2.27 | 5683 | 0.61 | 1514 | 2.69 | 6725 |
| 5.00% | % CV | 15 | 15 | 23 | 23 | 15 | 15 |
| D) | Mean | 26A | 1321 | 8.71 | 436 | 25.1 | 1757 |
| CT327CP4 | SD | 12.81 | 640 | 5.69 | 284 | 14.64 | 732 |
| 0.1% | % CV | 48 | 48 | 65 | 65 | 42 | 42 |

Efficiency of Delivery

The efficiency of delivery is characterized by expressing amount of drug permeating as a percent of applied dose.

### Dermal Deposition

Dermal deposition of CT327 from the evaluated formulations ranged from 1.46 to 8.71 percent of the applied dose. Formulations CT327OP1 (A) and CT327CP4 (D) had the highest efficiency of CT327 dermal deposition with 5.87 and 8.71 percent of the applied dose respectively. Formulation CT327OP2 (B) exhibited the least efficient delivery into the dermis.

### Epidermal Deposition

Epidermal deposition of CT327 from the evaluated formulations ranged from 13.0 to 26.4 percent of the applied dose. Formulations CT327OP1 (A) and CT327CP4 (D) had the highest efficiency of CT327 epidermal deposition with 17.5 and 26.4 percent of the applied dose, respectively.

Formulation CT327OP2 (B) exhibited the least efficient delivery into the epidermis.

Total Amount Delivered

### Dermal Deposition

The calculated CT327 dermal deposition ranged from 147 to 6,630 ng/cm². Formulations CT327CP4 (D) and CT327OP3 (C) generated the highest CT327 dermal deposition with 436 and 6,630 ng/cm², respectively. Formulation CT327OP1 (A) produced the lowest CT327 dermal deposition, 147 ng/cm².

Dermal levels of CT327 following 24 hour duration of topical exposure are presented in FIG. 1 “Calculated nanograms of active substance delivered per square centimeter of skin surface area (ng/cm²)”.

### Epidermal Deposition

The calculated CT327 epidermal deposition ranged from 437 to 37,354 ng/cm². Formulations CT327OP2 (B) and CT327OP3 (C) had the highest CT327 epidermal deposition with 3,244 and 37,354 ng/cm², respectively. Formulation CT327OP1 (A) generated the lowest CT327 epidermal deposition, 437 ng/cm².

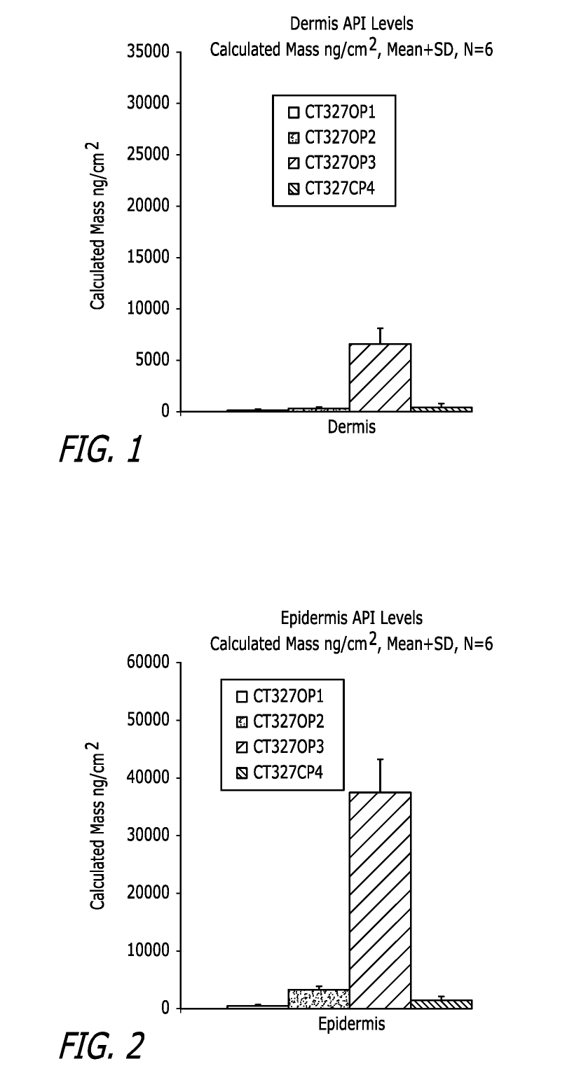

Epidermal levels of CT327 following 24 hour duration of topical exposure are summarized in FIG. 2 as “calculated nanograms of active substance delivered per square centimeter of skin surface area (ng/cm²).”

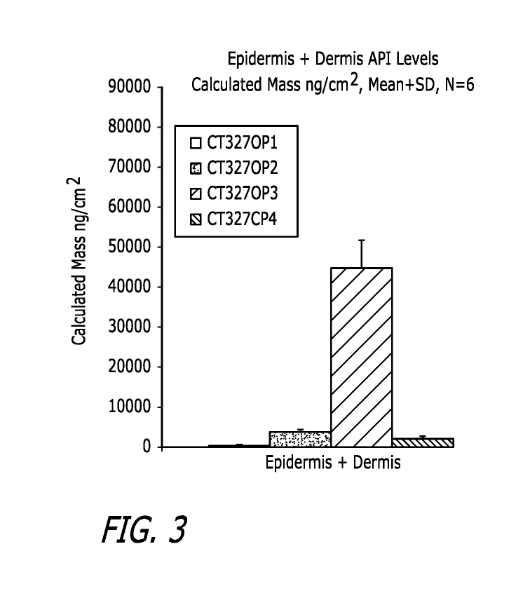

FIG. 3 presents the summed dermal and epidermal levels for all formulations used in this study.

### Statistical Analysis

Multiple T-Test analyses (see Tables 28, 29 and 30) indicated that the combined epidermis+dermis deposition observed with Formulae CT327OP1 (A), CT327OP2 (B), CT327OP3 (C), and CT327CP4 (D) were significantly different from each other.

TABLE 28
T-TEST STATISTICAL ANALYSIS OF
DERMAL LEVELS OF CT327 (NG/CM²)

| | To | | | |
|---|---|---|---|---|
| From | CT327OP1 | CT327OP2 | CT327OP3 | CT327CP4 |
| CT327OP1 | | | | |
| CT327OP2 | 0.002 | | | |
| CT327OP3 | 0.000 | 0.000 | | |
| CT327CP4 | 0.040 | 0.576 | 0.000 | |

TABLE 29
T-TEST STATISTICAL ANALYSIS OF EPIDERMAL
LEVELS OF CT327 (NG/CM²)

| | To | | | |
|---|---|---|---|---|
| From | CT327OP1 | CT327OP2 | CT327OP3 | CT327CP4 |
| CT327OP1 | | | | |
| CT327OP2 | 0.000 | | | |
| CT327OP3 | 0.000 | 0.000 | | |
| CT327CP4 | 0.008 | 0.000 | 0.000 | |

TABLE 30
T-TEST STATISTICAL ANALYSIS OF COMBINED EPIDERMIS
AND DERMIS LEVELS OF CT327 (NG/CM²)

| | To | | | |
|---|---|---|---|---|
| From | CT327OP1 | CT327OP2 | CT327OP3 | CT327CP4 |
| CT327OP1 | | | | |
| CT327OP2 | 0.000 | | | |
| CT327OP3 | 0.000 | 0.000 | | |
| CT327CP4 | 0.004 | 0.001 | 0.000 | |

### Conclusions

The purpose of this study was to characterize in vitro percutaneous absorption of CT327 from prototype formulations following application to excised human skin from elective surgery.

In vitro skin permeation data is intended to facilitate identification of formulation candidates with the highest potential of success, in terms of developing a topical formulation with appropriate delivery characteristics. This approach may also provide the highest potential for correlating in vitro permeation data with clinical efficacy, in diseases where the site of action is located in the viable tissues of the skin.

Data from this preliminary in vitro skin permeation experiment indicated that prototype CT327CP4 (D) exhibited the highest efficiency of delivery for CT327, while CT327OP3 yielded the highest tissue deposition of CT327. In terms of combined CT327 deposition into the epidermis and dermis, we see a dose dependent trend in the delivery profiles in ointment formulations. It should be noted that as the concentration of CT327 increases from 0.05% to 5.00% w/w, an increase in tissue deposition from 584 ng/cm² to 43,984 ng/cm² is observed. However, the cream prototype is more efficient in delivering CT327 than the ointment prototype.

In addition to the delivery potential of the prototypes, final prototype selection is expected to factor in other evaluation parameters such as physical and chemical stability of the formulations, disease state and availability of supporting toxicological data.

### Example 3: Stability Studies on SNA-120 (CT327) Topical Ointments (0.5% W/W, 0.1% W/W AND 0.05% W/W)

### Introduction

SNA-120 Drug Product is a homogeneous white to off white topical ointment manufactured by Patheon UK Ltd at three different strengths (0.5% w/w, 0.1% w/w, and 0.05% w/w). Patheon is responsible for the cGMP manufacture, quality control testing and release of active ointment batches and matching placebos. Patheon is also responsible for stability testing, following ICH guidelines, of SNA-120 Drug Product batches that were manufactured and used in different clinical trials.

Three different batches of each indicated strength of SNA-120 Topical Ointment were manufactured:

0.05% w/w: Batches PD 11187, PD 11294, and PD 12034

0.1% w/w: Batches PD11188, PD11295, and PD11298

0.5% w/w: Batches PD11189, PD11296, and PD11299

Matching placebo: Batches PD11186, PD11205, and PD11206

All batches were produced using the same manufacturing process. The container used was a 30 mL Amber glass jar, closed by a black screw top (urea/PVDC liner). Further information regarding each batch is provided in Table 31 below.

TABLE 31
SNA-120 OINTMENT BATCHES MANUFACTURED FOR THE SNA-
120 PHASE IIB PSORIARIS VULGARIS CLINICAL TRIAL

| Batch n. | Strength | Batch size | | Manufacturing Date |
|---|---|---|---|---|
| PD11186 | Placebo | 15 | kg | 22 Sep. 2011 |
| PD11187 | 0.05% | 15 | kg | 29 Sep. 2011 |
| PD11188 | 0.1% | 15 | kg | 10 Oct. 2011 |
| PD11189 | 0.5% | 15 | kg | 26 Oct. 2011 |
| PD11205 | Placebo | 10 | kg | 7 Nov. 2011 |
| PD11206 | Placebo | 12 | kg | 9 Nov. 2011 |
| PD11294 | 0.05% | 12.5 | kg | 12 Jan. 2012 |
| PD11295 | 0.1% | 12.5 | kg | 9 Feb. 2012 |
| PD11296 | 0.5% | 12.5 | kg | 5 Mar. 2012 |
| PD12034 | 0.05% | 9.5 | kg | 14 Mar. 2012 |
| PD11298 | 0.1% | 9.5 | kg | 20 Mar. 2012 |
| PD11299 | 0.5% | 9.5 | kg | 26 Mar. 2012 |

Batches PD11187, PD11188 and PD11189 were placed on an ICH stability study at three different conditions (5° C., 25° C./60% RH, and 40° C./75% RH) up to 68 months (pullpoints: 0, 1, 2, 3, 6, 9, 12, 18, 24, 36, 48, 60 and 68 months). The tests at each timepoint are summarized in Table 32 below.

TABLE 32
STABILITY TESTING PROGRAM
(BATCHES PD11186-PD11189)

| | | Storage Condition | | |
|---|---|---|---|---|
| Timepoint | | 5° C. | 5° C./60% RH | 40° C./75% RH |
| 1 | month | (A) | A | A |
| 2 | months | (A) | A | A |
| 3 | months | (A) | A | A |
| 6 | months | (A) | A | ABCD |
| 9 | months | (A) | A | — |
| 12 | months | (ABCD) | ABC(D) | — |
| 18 | months | (A) | A | — |
| 24 | months | (ABC) | ABC | — |
| 36 | months | (ABCD) | ABCD | — |
| 48 | months | (ABC) | ABC | — |
| 60 | months | (ABC) | A (C for PD11189) | — |
| 68 | months | — | A for PD11186 and | — |
| | | | PD11189 only | |

( ): optional testing in case of failure of a sample stored at a higher temperature
Legend:
A: Appearance (ointment and container) (Method Ref. ATP007) HPLC Assay and related substances Method Ref. ATP1078) Benzyl Alcohol assay (Method Ref. ATP1077)
B: Viscosity (Method Ref. ATP899)
C: Total Microbial Count, Absence of Ps. aeruginosa, Absence of S. aureus (Method Ref. USP<61>, USP<62>, Ph. Eur. 2.6.12, Ph. Eur. 2.6.13)
D: Preservative Efficacy Testing (PET)

The other six active batches (PD11294, PD11295, PD11296, PD12034, PD11298, and PD11299) were placed on a stability study at 25° C./60% RH (representative of likely in use storage conditions) up to 12 months (pullpoints: 0, 1, 2, 3, 6, and 12 months). In this case, the tests performed at each timepoint were limited to those which are indicative of shelf-life limiting parameters, as detailed in Table 33 below.

TABLE 33
STABILITY TESTING PROGRAM (PD11294-PD11296,
PD12034, PD11298 AND PD11299 BATCHES)

| | |
|---|---|
| | Storage condition |
| Timepoint | 5° C./60% RH |

| | | |
|---|---|---|
| 1 | month | A |
| 2 | months | A |
| 3 | months | A |
| 6 | months | A |
| 12 | months | A |

Legend:
A: Appearance (ointment and container) (Method Ref. ATP007) HPLC Assay and related substances Method Ref. ATP1078)

At each timepoint, the results of the testing are compared with the specifications submitted in the regulatory documents that were approved by the competent authorities.

### Results and Discussion

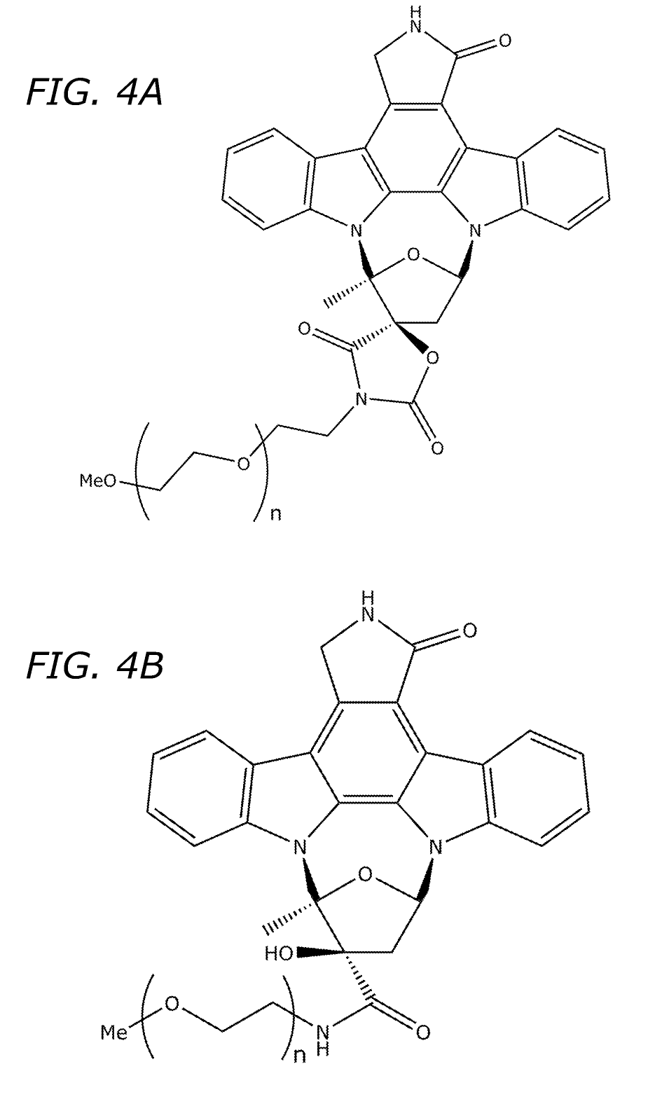

Results from an earlier stability study on the SNA-120 Topical Ointment formulation (0.5% w/w batch PD 11034) showed that the degradation of the active ingredient SNA-120 to the amide (reported as impurity RRT 0.40-0.47) is the main degradation pathway that occurs at ambient and accelerated temperatures, most probably due to the presence of water in the excipients and/or introduced from the environment. The structures of SNA-120 and the amide are shown in FIG. 4.

The degradation of SNA-120 to amide had been observed to occur to a much more significant level in an earlier clinical formulation (a water/oil emulsion containing cetostearyl alcohol, cetomacrogol 1000, white soft paraffin, liquid paraffin, glycerol, benzyl alcohol and 0.1% w/w SNA-120 in pH 4.0 citrate buffer). Indeed, the rate of degradation in this cream formulation was such that refrigerated storage and a high amide specification limit (6%) was used in order to obtain a practical shelf-life to support the clinical trials. These observations, together with a suggested preference for an ointment versus a cream in certain clinical indications, led to the development of an anhydrous topical ointment formulation, the stability of which is the focus of this Example. In the ointment formulation, amide formation is the limiting (stability-indicating) parameter, and a specification limit of NMT 3.0% was set for this.

Statistical analysis of stability data to determine the SNA-120 Drug Product shelf life is performed by regression analysis employing the “Macro Based FDA STAT Package For the Generation of Shelf Life Predictions” published by the Pharmaceutical Analytical Sciences Group (PASG) (http://www.pasg.org.uk/excel.htm). The statistical analysis determines the point at which the 90% upper confidence limit for the degradants exceeds the specified acceptance criteria.

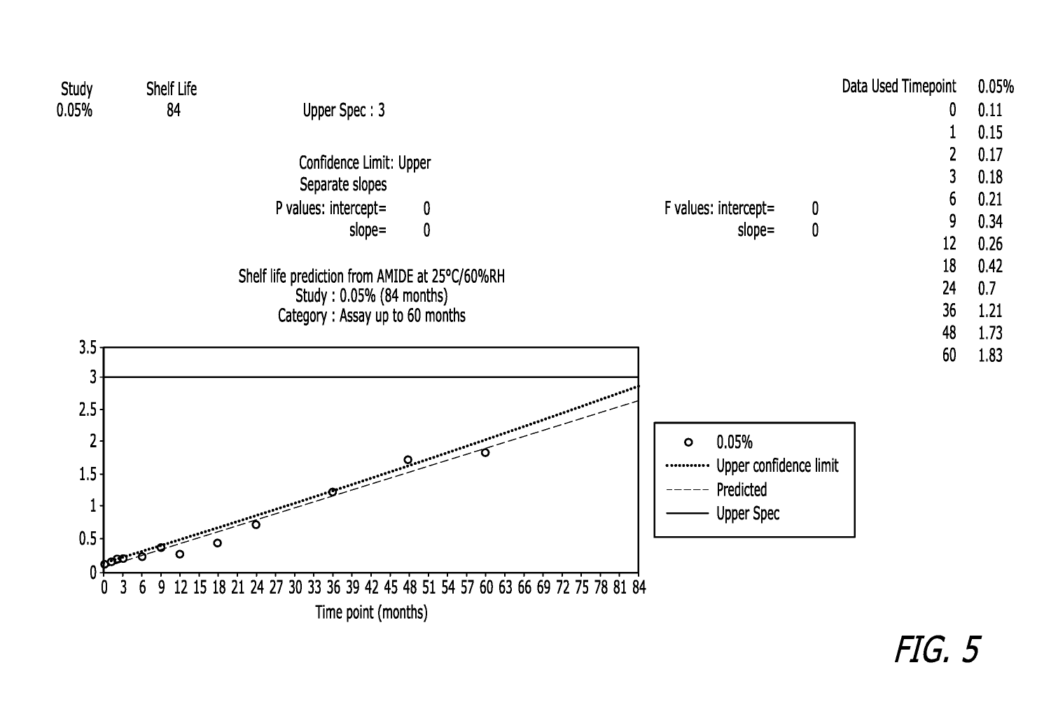

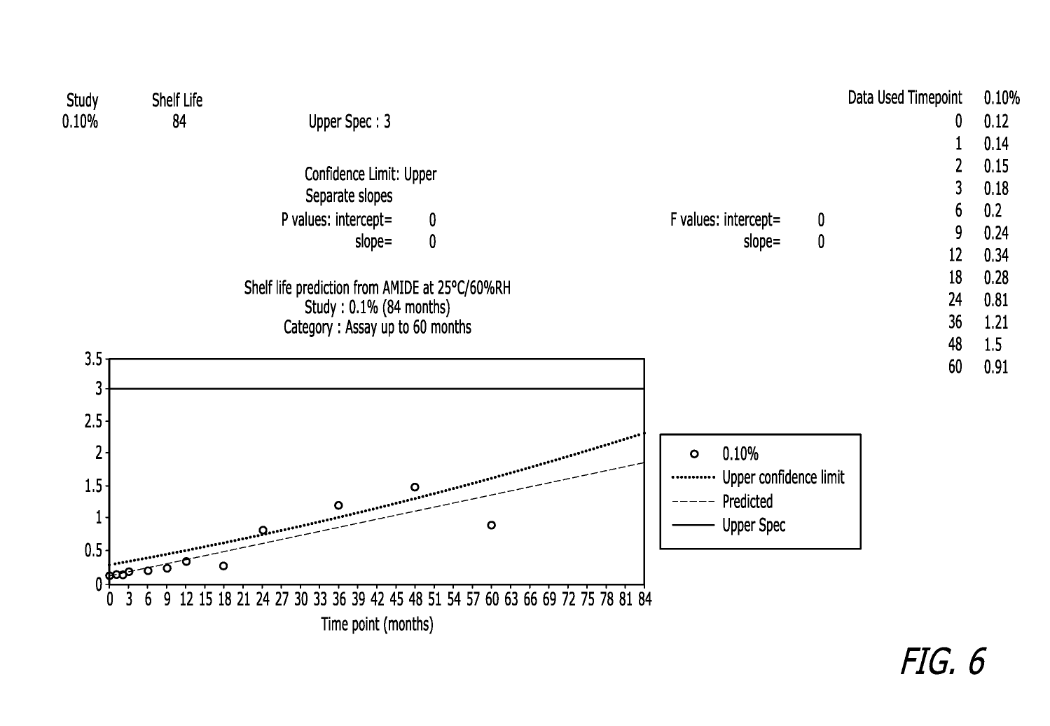

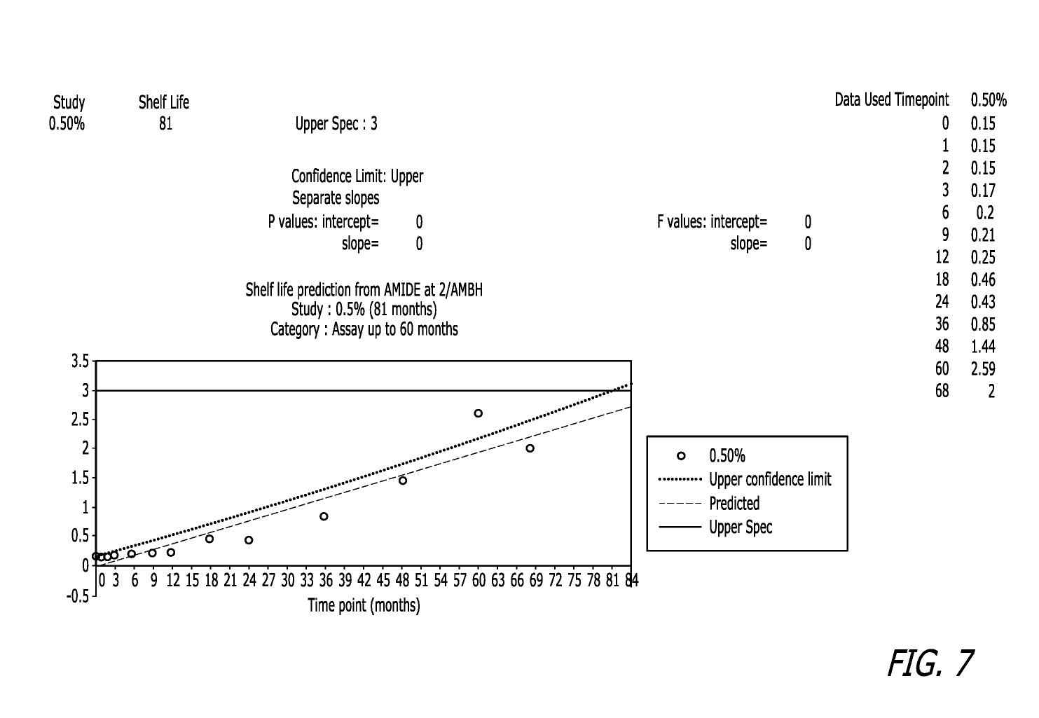

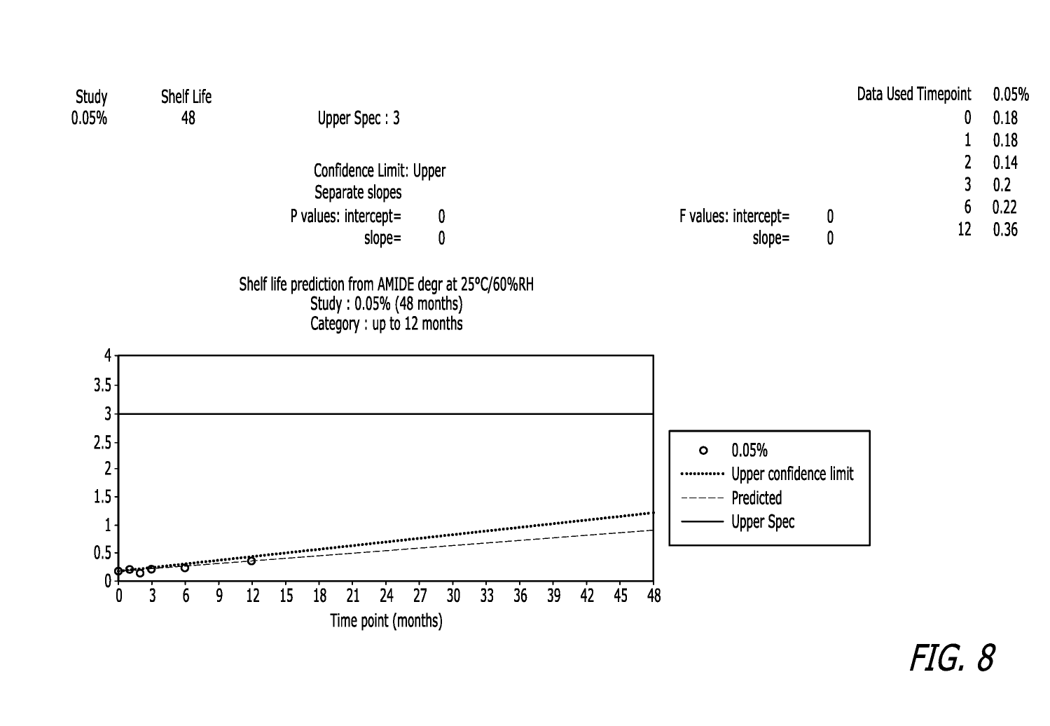

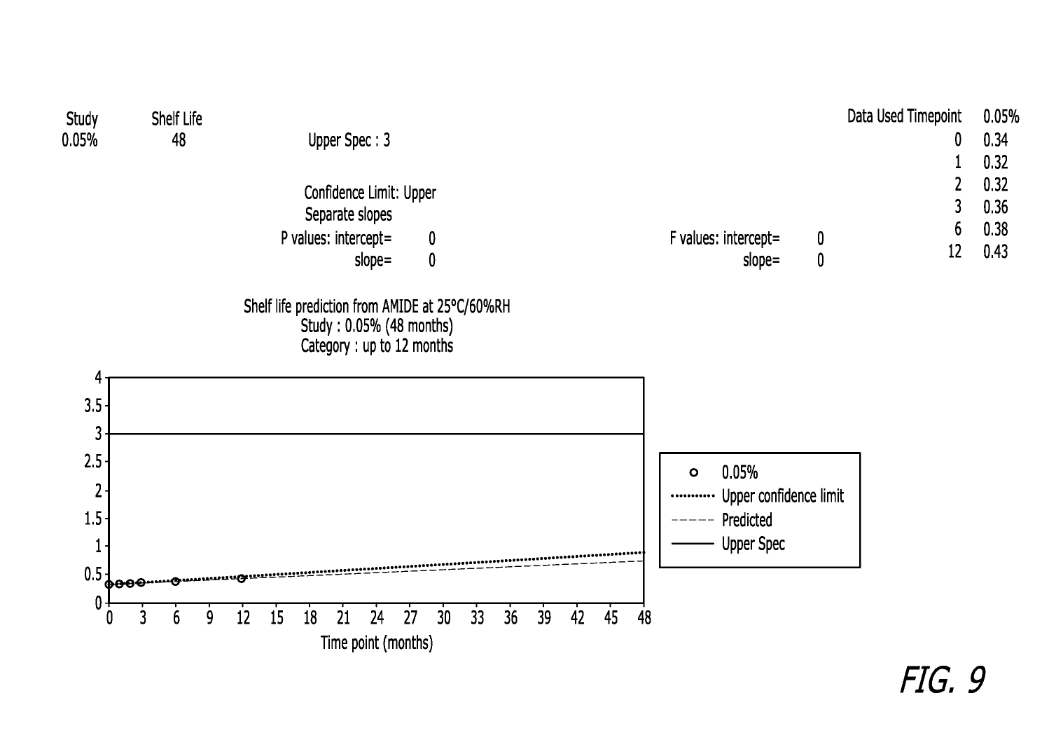

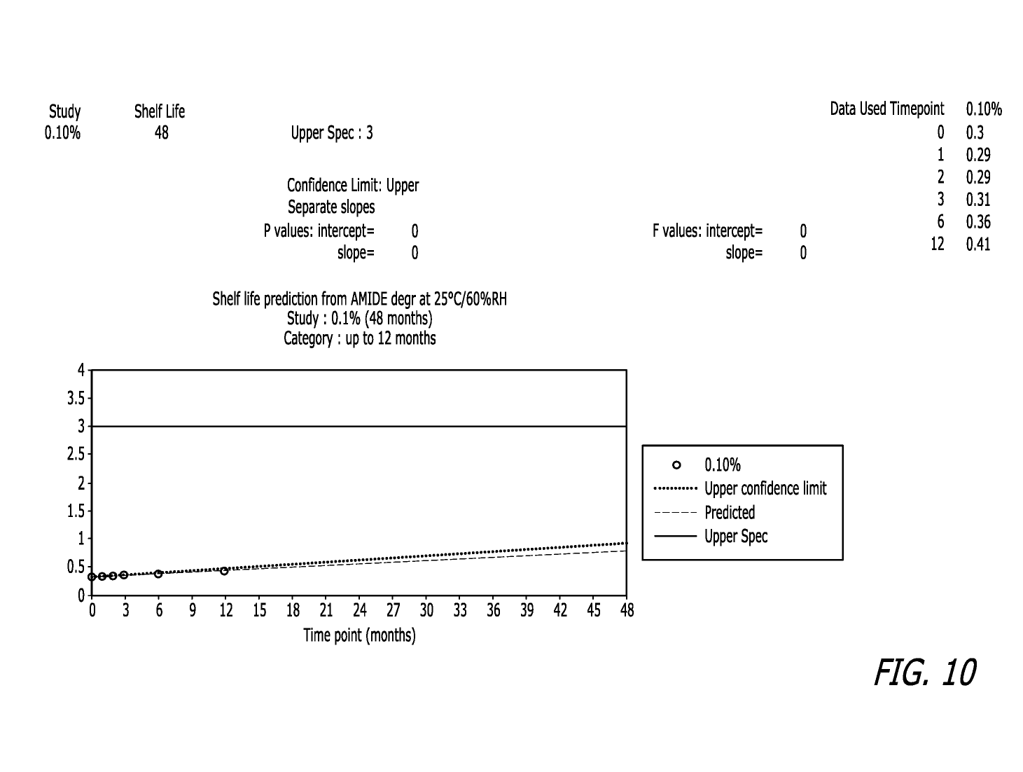

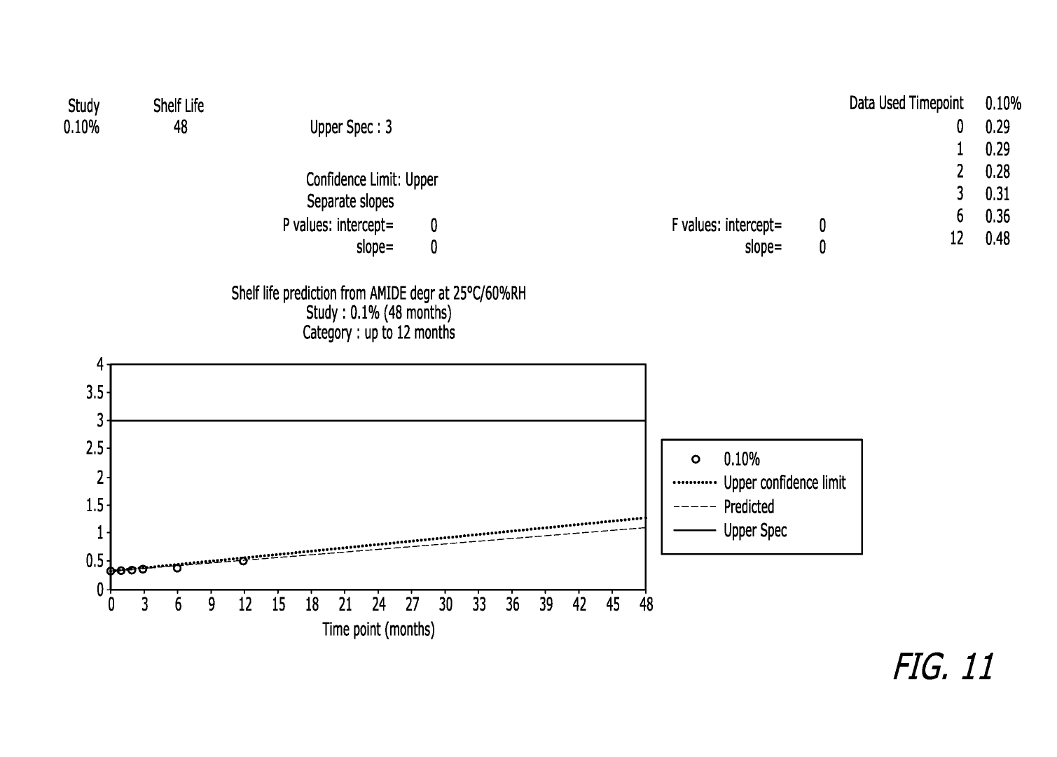

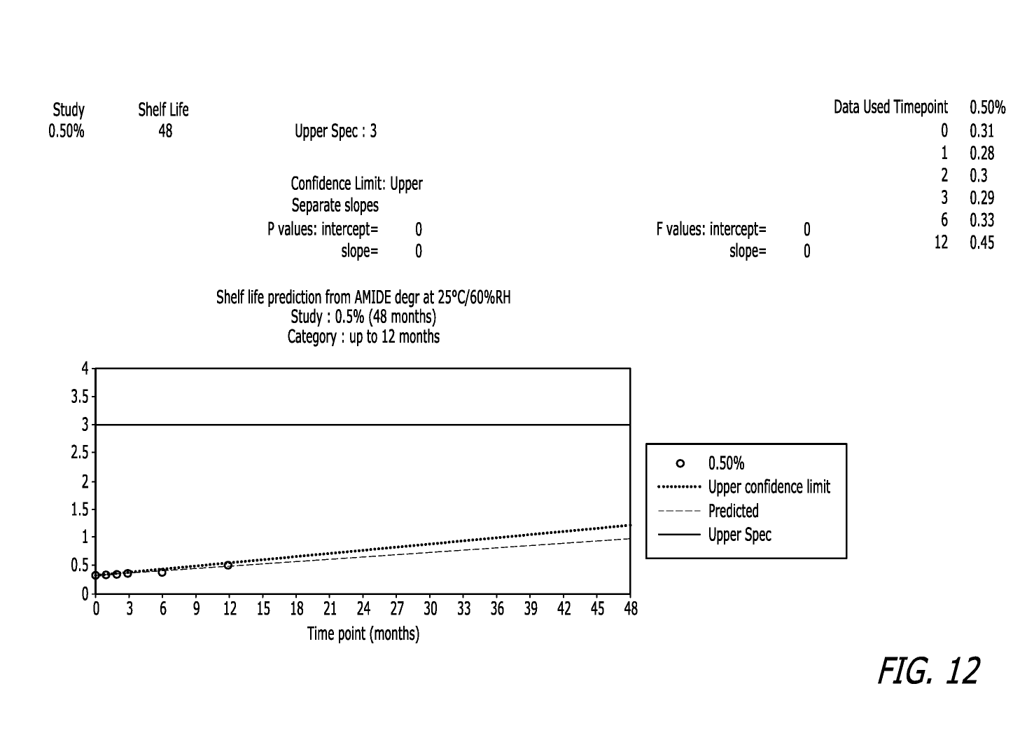

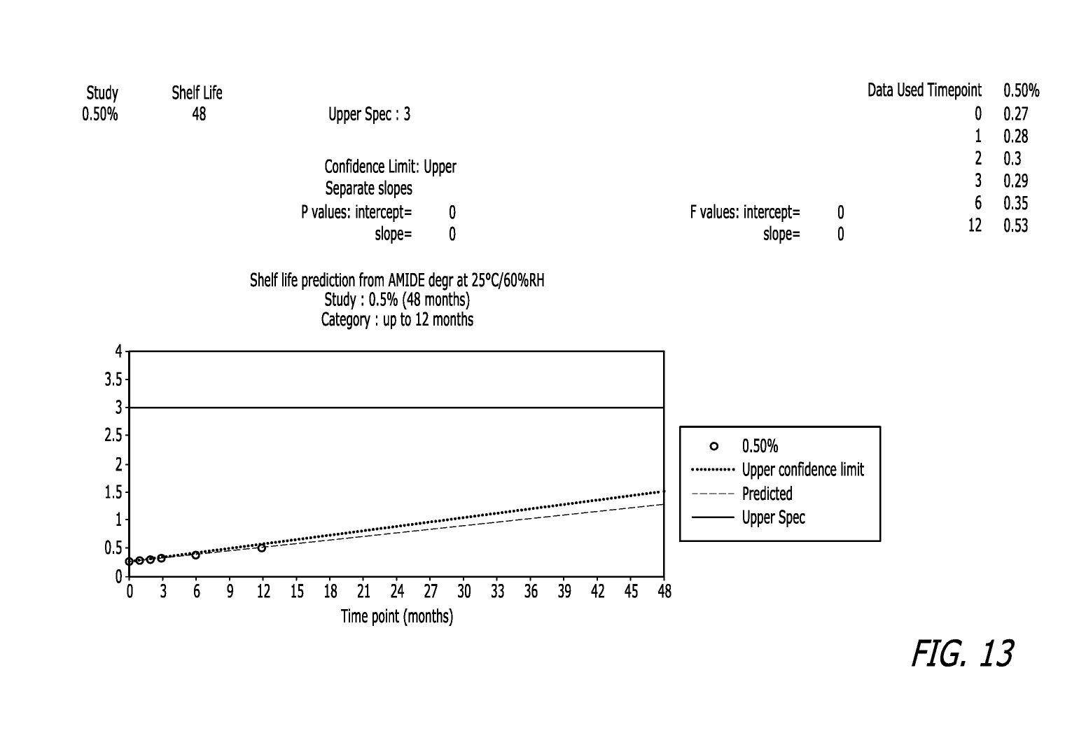

FIGS. 5 to 13 present shelf life predictions for each batch separately, using the Separate Slopes statistical model in the FDA STAT Package.

The FDA STAT programme can also analyse multiple batches together. In this case, the programme automatically selects the most appropriate model for any particular dataset, as summarised in the following extract from the FDA STAT User Guide.

Common Intercept and Common Slope:

If this model is selected then the data sets for each batch have similar values at the initial timepoint and a similar gradient, therefore the data is pooled. This means a single shelf life will be determined for all the batches being tested and the data will be plotted on a single graph.

Separate Intercepts and Common Slope:

This model indicates the data for all the batches is significantly different at the initial time point, but the gradients are similar. As a result, a shelf life is generated per batch, but the data from all the batches is pooled for the calculation of confidence intervals. This means a more accurate shelf life is generated, but it may differ from batch to batch. Each batch will be plotted on a separate graph.

separate slopes: if this model is chosen, then the data for all the batches being tested have significantly different gradients, as a result all the batches are tested completely separately and separate shelf lives and graphs are obtained.

It should be noted that the statistical model selected by the programme does not affect the resulting predicted shelf life of any particular batch, but it may provide further confidence in the prediction.

0.05% w/w, 0.1% w/w, and 0.5% w/w batches were analysed together (three batches of each strength) taking into account both all the available data (up to 68 months for PD11187-PD11189 batches, up to 12 months for the others) and just data up to 12 months (i.e. data from timepoints that are common to all the analyzed batches).

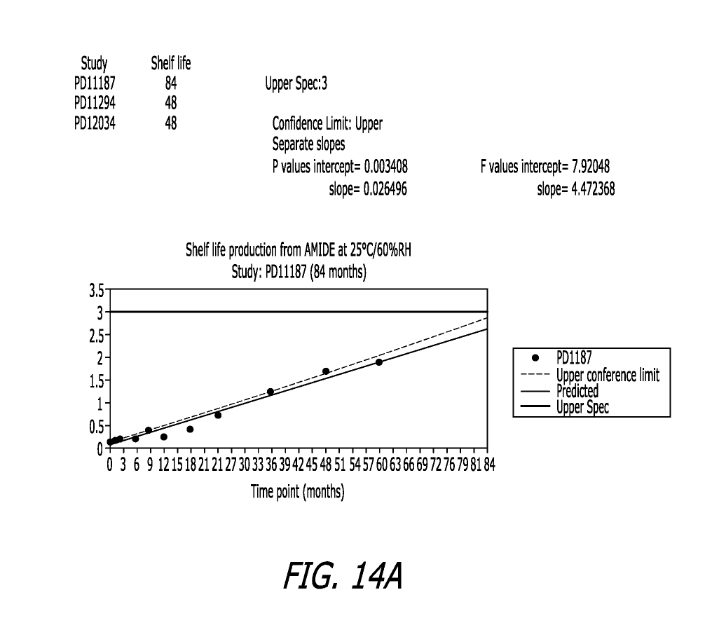

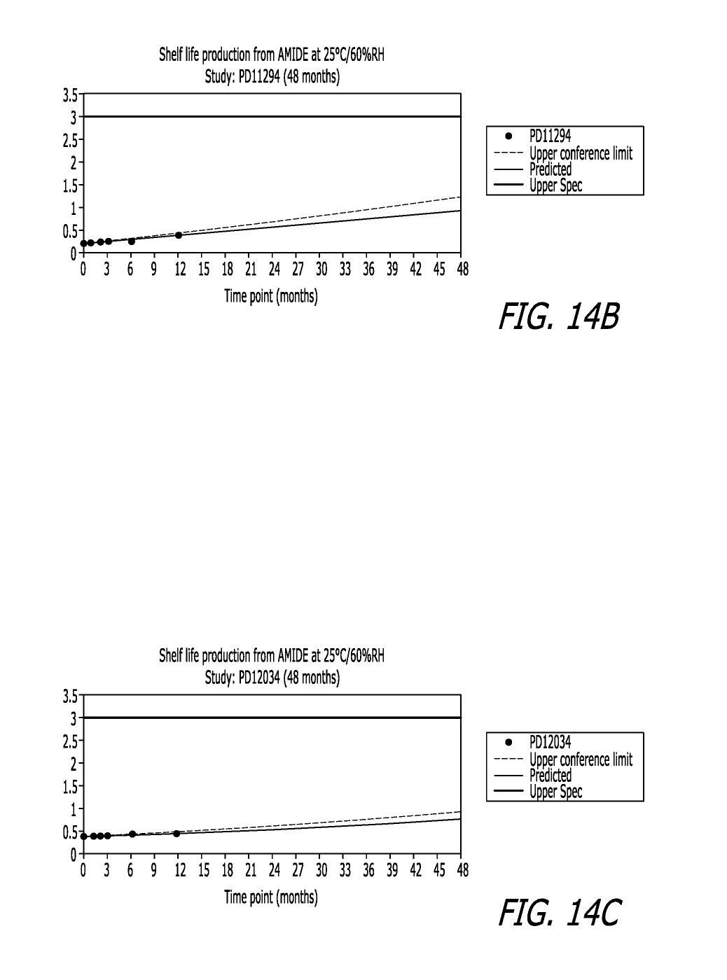

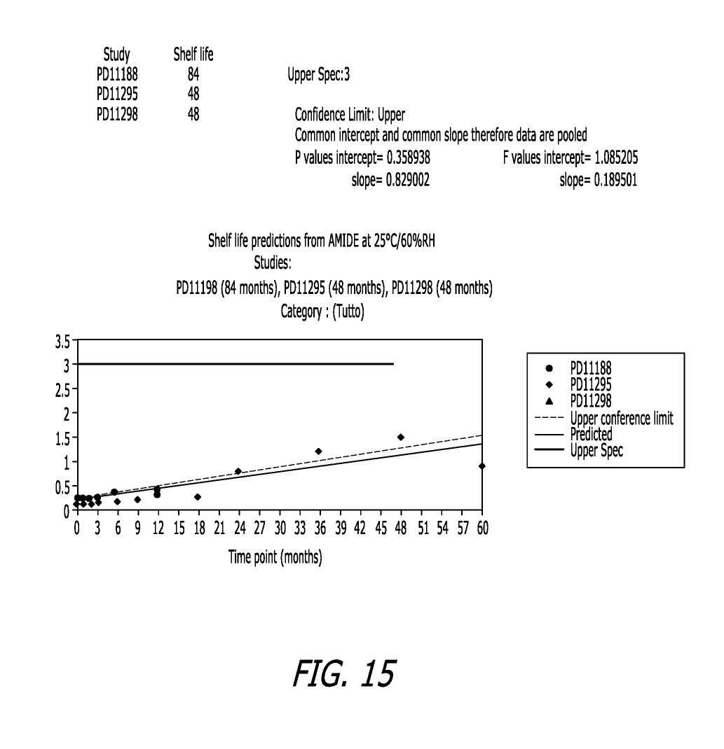

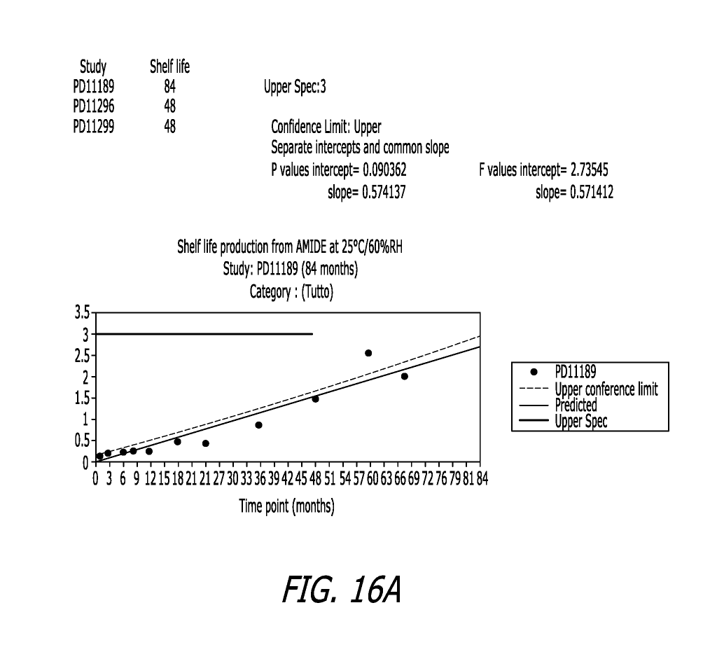

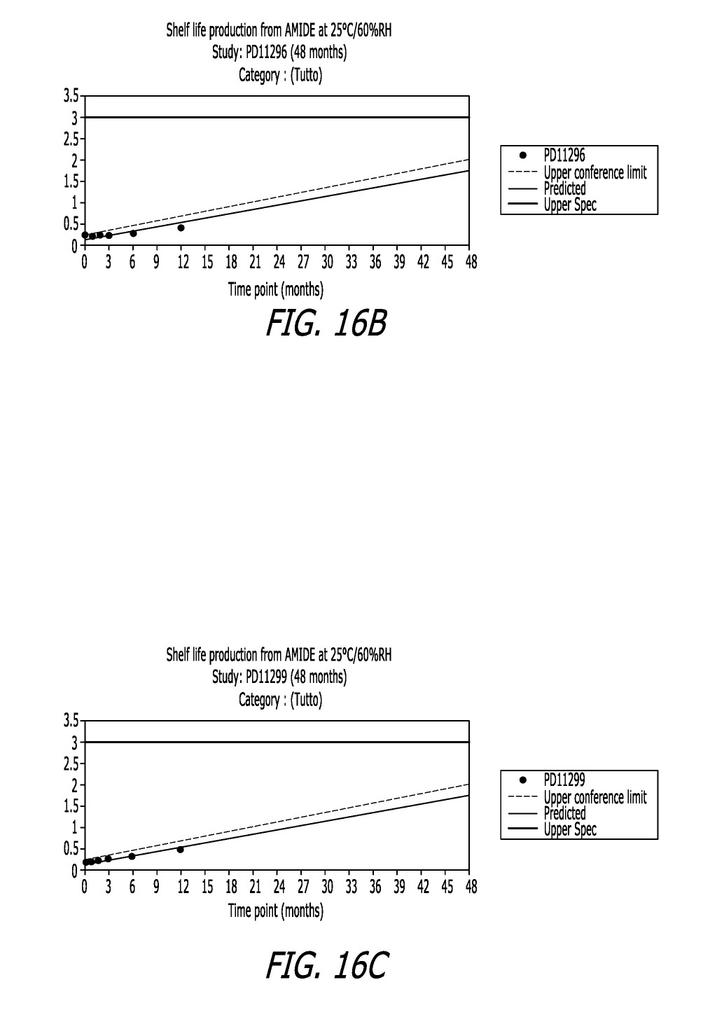

The resulting graphs showing shelf life predictions for each batch are presented in FIGS. 14 to 16.

Considering all the stability data available so far, the Separate Slopes statistical model was selected by the FDA STAT programme for the 0.05% w/w presentation, indicating that these data exhibit differences both at t-zero and in terms of gradient. This is a statistical observation, and the gradient differences are of little (if any) consequence in the context of the specifications and the likely long-term shelf-life assignment, as degradation to amide is very slow. On the other hand, the Separate Intercepts and Common Slope statistical model was selected by the FDA STAT programme for the 0.5% w/w batches, suggesting that these data exhibit significant differences at t-zero, but that the gradients are similar.

It should be emphasized that the FDA STAT package does not permit a shelf life prediction greater than four times the maximum time point in the data set; at present, and taking into account all data collected so far (up to 60-month data), we can assign a predicted shelf life at 25° C./60% RH of 84 months to batches PD11187 and PD11188, and of 81 months to batch PD11189 (considering data up to 68-month data), while a predicted shelf life at 25° C./60% RH of 48 months (from 12-month data) is assigned to batches PD11294, PD11295, PD11296, PD12034, PD11298 and PD11299.

However, it must be underlined that the above reported statistical processing refers to the main product-related degradant (SNA-120 amide) limit as key parameter for shelf life extrapolation. Unfortunately, 0.05% w/w batch n. PD11187 failed to meet assay criteria after 60 months at 25° C./60% RH, despite the amide impurity (related substance at RRT 0.40-0.47) being still in the acceptance criteria range. Such a low assay (0.42 mg/g), that was not confirmed at 5° C. (material still in spec after 60M), should not be a result of instability of the active material, as there is no significant increase in degradation at this timepoint. The most likely cause of the 00S would be inhomogeneity of the bulk material, being this a well-known issue for the 0.05% w/w ointment, as confirmed by an analogous below specification assay result observed following 3 months at 25° C./60% RH, with no corresponding increase in degradation.

From a regulatory perspective, according to ICH Guidance (01 E), a shelf-life assignment of double the real time long term stability data, up to a maximum extension of 12 months, is usually acceptable, provided that the long term and accelerated stability data support this, which in this case they clearly do.

At the time of manufacturing and release of the initial Phase IIb (CT327-2003) ointment batches (PD11186, PD11187, PD11188 and PD11189), this information was of course not available, and a shelf life of 9 months at ambient temperature was assigned to all new active batches, based on the stability data from the previous batch (PD11034), which is discussed further below. Emerging stability data with the Phase IIb clinical batches enabled the shelf life assignment to be subsequently extended up to the current 68 months for the placebo and 0.5% w/w ointment.

### Conclusions

Stability data collected on SNA-120 Topical Ointment at three different strengths (0.5% w/w, 0.1% w/w, and 0.05% w/w) has been summarized and discussed.

As part of this analysis and report, the shelf life limiting parameter is considered degradation of the active ingredient SNA-120 to the amide (RRT 0.40-0.47), for which the current upper specification limit is 3.0%.

All the analysed batches from the Phase lib (CT327-2003) manufacturing campaign show very limited degradation to the amide during the observation period (up to 60 months for batches PD11187, PD11188, up to 68 months for PD11189, and up to 12 months for PD11294, PD11295, PD11296, PD12034, PD11298 and PD11299).

At present, with the data collected so far, the FDA STAT statistical tool is able to assign a shelf life at ambient temperature of 84 and 81 months for batches P011187/PD11188 and P011189, respectively, and a shelf life at ambient temperature of 48 months for batches PD11294, P011295, PD11296, PD12034, P011298 and P011299. Although extrapolations readily show that further shelf-life extensions could be possible if additional data from subsequent time points were obtained, the absence of any future time points does not allow this extrapolation to be used. Therefore, a shelf life at ambient temperature of 68 months for the 0.5% strength and corresponding placebo is assigned based on real time stability data for batch P011189.

Batches PD11294, PD11295, PD11296, PD12034, PD11298 and PD11299 can be considered as representative of the proposed commercial product and manufacturing process, and it can therefore be concluded that SNA-120 Topical Ointment has excellent stability at ambient temperature.

The currently assigned shelf life for SNA-120 Topical Ointments, based on all of the available stability data and ICH guidance 01 E, is 60 months at ambient temperature for 0.1% w/w, 68 months for 0.5% w/w strength and placebo, and 48 months for 0.05% w/w strength.

### In Vivo Testing of SNA-120 Topical Ointment

SNA-120 topical ointment (0.5%) was shown to possess neither photoallergenic nor photoirritation potential following topical application to the skin in association with exposure to UV light in guinea pigs.

In addition, no systemic exposure could be detected following application of SNA-120 topical ointment (at doses varying from 0.05% to 0.5%) to the skin in human clinical trials.

### Example 4: SNA-120 Ointment Manufacturing Process Development

### Summary

A pilot lot of SNA-120 0.5% w/w formulation batch (300 g) was prepared as follows:

First, Phase A (Propylene Glycol, Benzyl Alcohol, BHT and SNA-120) and Phase B (White Petrolatum, White Wax, Stearyl Alcohol and Cholesterol) were weighed into separate beakers.

Second, Phase B was heated at 60° C.±5° C. until all excipients melted, with continuous propeller mixing (mixing speed 240 rpm for approx. 1 hour and 10 minutes); once it melted, it was cooled down below 45° C.

Third, Phase A was mixed with a stirrer bar for approximately 50 minutes at 240 rpm until homogeneous solution was achieved.

Fourth, when Phase B reached 40±5° C., both phases were then added together and mixed in order to get homogeneous dispersion of Phase A into Phase B. With continuous propeller mixing (1200 rpm for approx. 1 hour and 30 minutes) the mixture had cooled to room temperature.

This process was employed to manufacture a 500 g nonGMP placebo ointment batch and a 170 g nonGMP 0.5% w/w SNA-120 ointment batch using a Silverson mixer.

Further scale-up of the ointment manufacturing process was performed in the Krieger mixer. A 12 kg placebo ointment batch and a 7 kg 0.05% w/w SNA-120 ointment batch were manufactured, both of which confirmed the suitability of the Krieger mixer and thus the feasibility of the manufacturing process on scale-up. Good homogeneity was demonstrated in the 0.05% w/w batch. The Phase B temperature was subsequently adjusted from 60° C. to 70° C. to ensure all materials were thoroughly melted and mixed. An additional In Process Control (IPC), for benzyl alcohol homogeneity, was subsequently incorporated.

This manufacturing process was successfully applied in the cGMP manufacture of multiple 0.5% w/w, 0.1% w/w, 0.05% w/w and placebo ointment batches, all of which met specifications and were released for use in a Phase 2b clinical study conducted in the UK and the USA.

### Batch Formula

The percentages of excipients and drug substance for the 0.5% w/w presentation that is proposed for use in the Phase 1 clinical study, are listed in Table 34.

TABLE 34
QUANTITATIVE COMPOSITION
OF SNA-120 BATCH PD13065

| | 0.5% w/w ointment | Batch |
|---|---|---|
| Active/Excipient | (composition % w/w) | Quantity (g) |
| BHT | 0.10 | 14.995 |
| (ButylatedHydroxyToluene) | | |
| Benzyl Alcohol | 5.00 | 749.73 |
| Propylene Glycol | 5.40 | 807.62 |
| White Petrolatum | 75.00 | 11246.0 |
| White Wax | 8.00 | 1199.57 |
| Stearyl Alcohol | 3.00 | 449.84 |
| Cholesterol | 3.00 | 449.84 |
| SNA-120 (CT327) | 0.50 | 77.06* |
| Total | 100% | 14994.655 |

*This amount includes a correction factor of 1.028 (taking into account SNA-120 purity of 97.3%).

### Description of Manufacturing Process and Process Controls

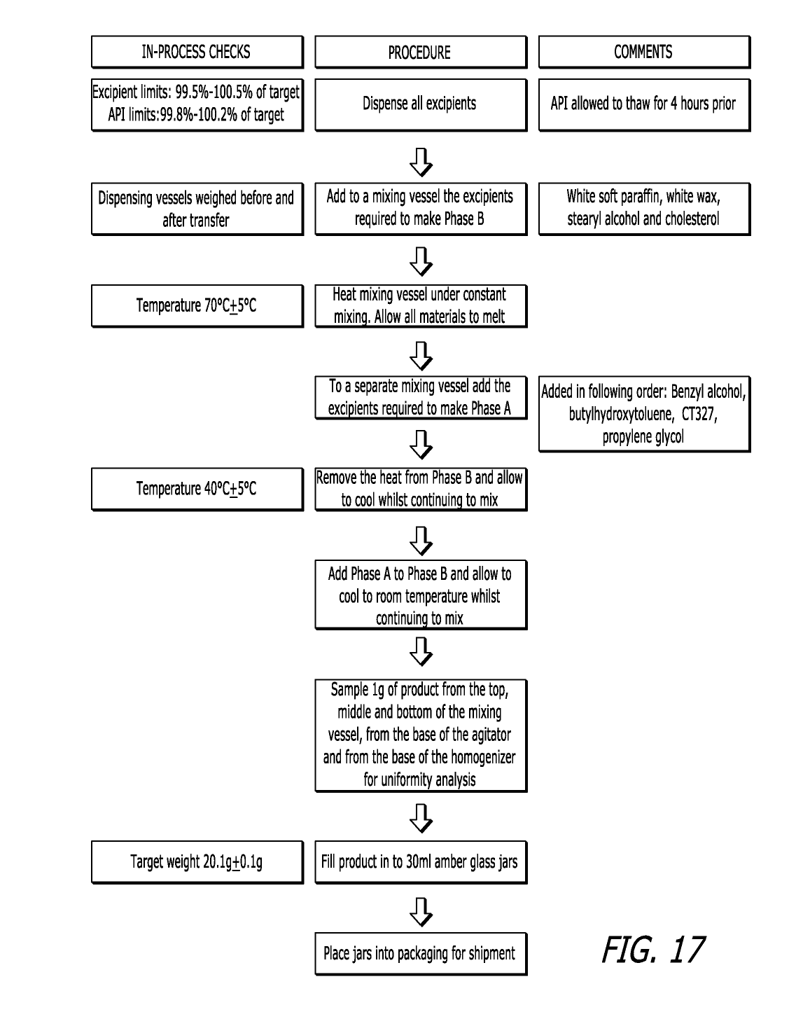

A flow diagram for the manufacture of a batch of SNA-120 ointment formulation is depicted in FIG. 17.

Description of Manufacturing Process

Preparation of Phase B

First, weigh the proper amount of white soft paraffin, white wax, stearyl alcohol and cholesterol and then transfer them into an appropriate vessel. Next, heat to 70° C.±5° C., whilst continuously mixing (at 25 rpm) using the Krieger mixer, to allow all the excipients to melt.

Preparation of Phase A

Weigh benzyl alcohol, butylhydroxytoluene, SNA-120 and propylene glycol. Hold approximately one-third of the propylene glycol in reserve. Transfer the benzyl alcohol to an appropriate vessel. Under constant stirring (25 rpm) add the remaining excipients in the following order: butylhydroxytoluene, SNA-120 and then propylene glycol.

Phase A and Phase B Mixing

Allow Phase B to cool to below 45° C. by turning off the heat whilst continuing to mix. When the Phase B reaches 40° C.±5° C., add Phase A to Phase B with continuous mixing in order to get homogenous dispersion. Use the propylene glycol held in reserve to wash out the Phase A mixing vessel and add these washings to the Phase A/Phase B mixture. Then allow the mixture to cool at room temperature with continuous propeller mixing then stop mixing.

Control of Steps and Intermediates

The following control points have been identified in the manufacturing process:

First, achievement of 70° C.±5° C. temperature after the addition of all the excipients to make Phase B in order to promote melting of excipients.

Second, reduction of the bulk temperature to 40° C.±5° C. prior to the final addition of the Phase A.

Third, controlled addition of Phase A to Phase B to facilitate distribution of the Phase A concentrate within the Phase B bulk matrix.

Fourth, agitation speed and time.

Prior to the filling of final SNA-120 ointment into jars, the bulk is monitored for appearance, SNA-120 content and homogeneity. The homogeneity in-process check is performed by taking and testing samples taken from top, middle and bottom of the mixing vessel, from the base of the agitator on its removal, and from the base of the homogenizer on its removal.

### Example 5: SNA-120 0.5% Ointment Specification and Characterization

### Summary

The specification for SNA-120 0.5% w/w drug product ointment is presented in Table 35 below.

TABLE 35
SNA-120 0.5% W/W OINTMENT SPECIFICATION

| TEST No | TEST | SPECIFICATION |
|---|---|---|
| 1 | DESCRIPTION | |
| 1.1 | Container and closure | Type III Amber glass jar/black urea/PVDC screw |
| | system | cap lid |
| | (ATP007) | |
| 1.2 | Appearance | Homogenous white to off white ointment |
| | (ATP007) | |
| 2 | IDENTITY | |
| 2.1 | CT327 HPLC | Conforms to reference material (RT +/− 10%) |
| | (ATP007) | |
| 2.2 | Preservative | Presence of Benzyl Alcohol |
| | (ATP1077) | |
| 3 | PROPERTIES | |
| 3.1 | Viscosity | To be reported |
| | (EP 2.2.10/USP <911>) | |
| 3.2 | Density | To be reported |
| | (EP 2.2.5 | |
| 4 | ASSAY AND RELATED SUBSTANCES | |
| 4.1 | Benzyl Alcohol assay | 4.5% to 5.5% w/w |
| | (ATP1077) | |
| 4.2* | Related substances by | All individual related substances ≥0.1% are to be |
| | RRT | reported |
| | (ATP1078) | RRT 0.40-0.47 ≤1.0% (release limit) |
| | | RRT 0.40-0.47 ≤3.0% (shelf-life limit) |
| | | No other single related substance ≥0.5% |
| 4.3 | Total sum of related | ≤2.0% (release limit) |
| | substances | ≤4.0% (shelf-life limit) |
| | (ATP1078) | |
| 4.4 | SNA-120 (CT327) assay | 4.50 mg/g-5.50 mg/g (release limit) |
| | (ATP1078) | 4.25 mg/g-5.50 mg/g (shelf-life limit) |
| 5 | MICROBIOLOGICAL TESTS | |
| 5.1 | Total microbial count | TAMC (Total Aerobic Microbial Count): ≤10² |
| | (EP 2.6.12/USP <61>) | cfu/g (maximum acceptable count = 200 cfu/g) |
| 5.2 | Absence of | Absent in 1 g |
| | Pseudomonas aeruginosa | |
| | (EP 2.6.13/USP <62>) | |
| 5.3 | Absence of | Absent in 1 g |
| | Staphylococcus aureus | |
| | (EP 2.6.13/USP <62>) | |

*RRT expressed as a time range due to the nature of PEGylated compounds

### Analytical Procedures

The following tests carried out on the SNA-120 ointment are pharmacopoeial methods: Viscosity (USP<911>/EP 2.2.10); Density (EP 2.2.5) and Microbiological Tests (USP<61>/EP 2.6.12/USP<62>/EP 2.6.13).

Summaries of the non-compendial methods are provided below. Performance of the methods is verified in each run via a system suitability check, and sample results are considered to be valid and can be reported when these criteria are met.

Test No. 1—Description

The descriptions of the container closure system and the ointment are determined by visual observation.

Test No. 2—Identity—Benzyl Alcohol Preservative

The identity of the benzyl alcohol preservative is determined by comparing the HPLC retention time of the benzyl alcohol peak in the sample solution to that of the benzyl alcohol standard solution. The result is reported as “Presence of Benzyl Alcohol” when the HPLC retention times match (+/−10%).

Test No. 3—Identity—SNA-120

The identity of the SNA-120 is determined only in the active ointment formulations by comparing the HPLC retention time of the SNA-120 peak in the sample solution to that of the SNA-120 standard solution. The result is reported as “Conforms to Standard” when the HPLC retention times match (+/−10%).

Test No. 4—Benzyl Alcohol Assay

The assay of the benzyl alcohol preservative is determined using a reversed phase HPLC method with UV detection at 254 nm. The assay of benzyl alcohol is determined on a weight/weight basis against an external benzyl alcohol standard. Benzyl alcohol is separated from SNA-120 and related substances using gradient elution on a phenyl column.

Test No. 5—SNA-120 Assay and Related Substances

A stability indicating reversed phase HPLC method with UV detection at 292 nm is used to determine the assay of SNA-120 during release and stability testing. The assay of SNA-120 is determined on a weight/weight basis against an external SNA-120 reference standard. The primary analyte, SNA-120, is separated from related impurities and potential degradants using gradient elution on a C18 column.

The stability indicating nature of the method was verified with stressed samples. Stress conditions included heat, heat/humidity, light, acid, base and oxidation. The peak purity of SNA-120 was checked for all the stressed samples and no evidence of co-elution with SNA-120 was observed.

The same HPLC method used to determine the assay is also used for determination of related substances. This method is capable of separating all potential degradants from each other and from SNA-120. The percentage of each impurity is calculated by comparing the impurity peak area to the peak area of SNA-120 and all impurities. The total impurities are determined by summing the individual organic impurities that are at or above the reportable limit (0.10% for 0.5% w/w ointment).

Validation of Analytical Procedures

Analytical validation studies on SNA-120 0.5% w/w topical ointment are presented below.

Validation of the HPLC Assay and Related Substances for CT327 Ointment

Validation Protocol

An HPLC method was developed for the detection of assay and related substances of SNA-120 drug product and placebo. The following method validation parameters were challenged: specificity; linearity; accuracy; precision (system and repeatability); range; stability of test solutions; limit of detection and quantitation; system suitability. Table 36 depicts a summary of the acceptance criteria.

TABLE 36
SUMMARY OF ACCEPTANCE CRITERIA

| Parameters | Acceptance Criteria |
|---|---|
| Specificity | No interference >2% with the quantification of the SNA-120 peak is |
| | observed from the sensitivity, diluent or placebo. |
| | The main degradants are resolved from the SNA-120 peak in the |
| | working standard. Any interference with the SNA-120 peak degradation |
| | products must be ≤ reporting threshold (sensitivity solution at 0.02 |
| | μg/ml) |
| Linearity | The acceptance criterion is correlation coefficient R ≥ 0.99. |
| Accuracy | Each individual recoveries must be between 97.0 and 103.0% |
| | The mean of all nine recoveries must be between 98 and 102% |
| System | Confirmation that the percent relative standard deviation (% RSD) of the |
| Precision | SNA-120 peak areas is ≤2.0%. |
| Repeatability | The % RSD for assay must be ≤3.0%. |
| Precision | The % RSD for related substances ≤0.5% area must be ≤20% |
| | The % RSD for related substances >0.5% area must be ≤15% |
| Range | The range will be reported as the range over which acceptable accuracy, |
| | precision and linearity is obtained. |
| Stability of | The concordance for stored standard and sample solutions should be |
| Test Solutions | within the range 97.0-103.0%. |
| | Related substance should not change by more than 0.1% area. |
| LOQ | The limit of quantitation for SNA-120 will be defined as the |
| | concentration that produces a signal to noise ration of ≥10. |
| LOD | The limit of detection for SNA-120 will be defined as the concentration |
| | that produces a signal to noise ration of ≥3. |
| System | All system suitability data collected throughout the validation will be |
| Suitability | collated and recommendations made regarding system suitability criteria |
| | to be set in the final method. |

Validation Results

Table 37 below depicts a summary of the validation data.

TABLE 37
SUMMARY OF VALIDATION DATA

| Parameters | Acceptance Criteria |
|---|---|
| Specificity | No interference was observed with the SNA-120 from the diluent or the |
| | placebo. The SNA-120 peak was resolved from degradants in the |
| | working standard and therefore met the acceptance criteria |
| Linearity | The correlation co-efficient (R) of 1.00 over the range 0.02 to 30 μg/ml |
| Accuracy | 50-150% of nominal |
| System | The % RSD of 0.2% for the SNA-120 ointment peak meets the |
| Precision | acceptance criteria |
| Repeatability | The % RSD (n = 6) for assay was 1.6% |
| Precision | The % RSD (n = 6_ for the three related substances recorded at below |
| | 0.5% area were ≤20% |
| Range | 9 to 28 μg/ml |
| Stability of | 0.5% w/w/formulation solution stability: |
| Test Solutions | Standards are stable for up to 3 days when protected from light at |
| | ambient conditions. |
| | Samples are stable for up to 20 hours when stored protected from the |
| | light at ambient conditions. |
| LOQ | The LOD solutions met the signal noise acceptance criteria. The LOD is |
| | confirmed as 0.05% of nominal |
| LOD | The LOQ solutions met the signal noise acceptance criteria. The LOQ is |
| | confirmed as 0.1% of nominal |
| System | System suitability parameters confirmed |
| Suitability | |

### Conclusion

The method for the determination of SNA-120 drug substance content and related substances in SNA-120 drug product and placebo has been successfully validated for its intended use.

CT327 Validation of the HPLC Method for Determination of Benzyl Alcohol Content in CT327 Ointment and Placebo

Validation Protocol

An HPLC method was developed for the detection of Benzyl Alcohol in SNA-120 drug product and placebo. The following method validation parameters were challenged: specificity; linearity; accuracy; precision (system and repeatability); range; stability of test solutions; system suitability. Table 38 depicts a summary of the acceptance criteria.

TABLE 38
SUMMARY OF ACCEPTANCE CRITERIA

| Parameters | Acceptance Criteria |
|---|---|
| Specificity | No interference >2% with the quantification of the benzyl |
| | alcohol peak is observed from the reference diluent, |
| | placebo, benzaldehyde or benzoic acid peaks. |
| Linearity | The acceptance criterion is correlation coefficient R ≥ |
| | 0.99. |
| Accuracy | Each individual recovery must be between 95.0 and 105.0% |
| | The mean of all nine recoveries must be between 97 and |
| | 103% |
| System | Confirm that the percent relative standard deviation |
| Precision | (% RSD) of the benzyl alcohol peak areas is ≤2.0%. |
| Repeatability | The % RSD of all recoveries must be ≤4.0%. |
| Precision | |
| Range | The range will be reported as the range over which |
| | acceptable accuracy, precision and linearity is obtained. |
| Stability of | The concordance of the benzyl alcohol in the standard |
| Test | solution should be within 97.0-103.0%. |
| Solutions | |
| System | All system suitability data collected throughout the |
| Suitability | validation will be collated and recommendations made |
| | regarding the system suitability criteria to be set in |
| | the final method. |

Validation Results

Table 39 below depicts a summary of the validation data.

TABLE 39
SUMMARY OF VALIDATION DATA

| Parameters | Results |
|---|---|
| Specificity | Peak <2% of the normal peak area, met the acceptance |
| | criteria |
| Linearity | The correlation co-efficient. (R) of 1.00 over the range |
| | 0.10 to 0.30 mg/ml benzyl alcohol in solution |
| Accuracy | 50-150% of nominal |
| System | The % RSD of 0.1% for the benzyl alcohol peak meets |
| Precision | the acceptance criteria |
| Repeatability | The % RSD for all 6 determinations is 1.7% meets the |
| Precision | acceptance criteria |
| Range | The range of the method is confirmed as 0.10 to 0.30 |
| | mg/ml over accuracy, precision and linearity was obtained. |
| Stability of | Standards are stable for up to 10 days when stored at |
| Test | ambient conditions. Samples are stable for up to 2 days |
| Solutions | when stored at ambient conditions. |
| System | System suitability parameters confirmed |
| Suitability | |

### Conclusion

The method for the determination of benzyl alcohol content in SNA-120 drug product and placebo has been successfully validated for its intended use.

Validation of the Microbiological Test

Validation Protocol

The validation study on microbiological test is accordance with the Ph Eur and USP methods which are harmonised in accordance with Ph Eur 2.6.12 and 2.6.13 method B and USP <61> and <62>. The study has also been validated for preservative efficacy in accordance with Ph Eur 5.1.3 and USP <51> as a category 2 product (Topical product). Table 40 depicts a summary of the acceptance criteria.

TABLE 40
SUMMARY OF ACCEPTANCE CRITERIA

| Parameters | Acceptance Criteria |
|---|---|
| Total Viable | Total Aerobic Microbial Count (TAMC): ≤10² cfu/g |
| count | (maximum acceptable count = 200) |
| | Total Count Yeasts and Mould (TCYM): ≤10¹ cfu/g |
| | (maximum acceptable count = 20) |
| Absence of | Pseudomonas aeruginosa Absent in 1 g |
| specified | Staphylococcus aureus Absent in 1 g |
| organisms | |
| Negative | No growth |
| control | |
| Preservative | Bacteria: |
| efficacy test | 2 days - 2 from initial count |
| | 7 days - 3 from initial count |
| | 14 days - 2 from initial count |
| | 28 days - no increase from day 7 count |
| | Fungi: |
| | 14 days - 2 from initial count |
| | 28 days -no increase from day 14 count |

Validation Protocol

Table 41 below depicts a summary of validation data.

TABLE 41
SUMMARY OF VALIDATION DATA

| Parameters | Results |
|---|---|
| Total Viable | A count of any of the test organisms differing by not more |
| count | than a factor of 2 from the positive control is to be |
| | obtained |
| Absence of | Any of the test organisms recovered from both the positive |
| specified | control and test samples |
| organisms | |
| Negative | No growth |
| control | |
| Preservative | A count of any of the test organisms differing by not more |
| efficacy test | than a factor of 2 (50%) from the positive control is to be |
| | obtained. |
| | Organism identifications confirm the inoculum. |
| | Positive control: <100 Colony Forming Units (CFU) |

### Conclusion

The method for the Microbiological Tests for SNA-120 drug product and placebo has been successfully validated for its intended use.

Batch Analyses

Analysis of the SNA-120 0.5% w/w ointment batches depicted in Table 42 is presented below in Tables 43-46.

TABLE 42
SUMMARY OF SNA-120 0.5% W/W
OINTMENT BATCHES ANALYZED

| | Date of | | | |
|---|---|---|---|---|
| Batch Number | Manufacture | Batch Size | | Purpose |
| MPF11006 | 18 Feb. 2011 | 170 | g | Feasibility batch |
| PD11034 | 30 Mar. 2011 | 1 | kg | Clinical Phase 1b |
| PD11189 | 26 Oct. 2011 | 15 | kg | Clinical Phase 2b |
| PD11296 | 5 Mar. 2012 | 12.5 | kg | Clinical Phase 2b |
| PD11299 | 26 Mar. 2012 | 9.5 | kg | Clinical Phase 2b |
| PD13063 | 28 May 2013 | 15.0 | kg | Clinical Phase 2b |
| PD13064 | 28 Jun. 2013 | 15.0 | kg | Clinical Phase 2b |
| PD13065 | 4 Jul. 2013 | 15.0 | kg | Clinical Phase 2b |

TABLE 43
FEASIBILITY BATCH MPF11006

| Test | Result | |
|---|---|---|
| Appearance | Homogeneous off- | |
| | white ointment | |
| CT327 Assay (HPLC-UV) | 5.10 | mg/g |
| Benzyl Alcohol Assay | 5.7% | w/w |
| (HPLC-UV) | | |

| | |
|---|---|
| Impurities (HPLC-UV) | |
| RRT 0.40-0.46 | 0.27% |
| RRT 1.49 | 0.17% |
| RRT 2.00 | 0.12% |
| RRT 2.75-2.76 | ND |

ND: Not Detected

TABLE 44
CLINICAL PHASE 1B BATCH PD11034

| Test | Specification | Result | |
|---|---|---|---|
| Appearance | Homogeneous off- | Homogeneous off- | |
| | white ointment | white ointment | |
| Identity | Conforms to reference | Conforms to | |
| (HPLC-UV) | material (RT ± 10%) | reference material | |
| Preservative | Presence of | Present | |
| | Benzyl Alcohol | | |
| Viscosity | To be reported | 1042000 | cP |
| Extractable weight | 20.0 g to 20.2 g | 20.0 | g |
| Density | To be reported | 0.892 | g/cm³ |
| Benzyl Alcohol | 4.5% to 5.5% w/w | 4.9% | w/w |
| Assay (HPLC-UV) | | | |
| CT327 Assay | 4.50 mg/g-5.50 mg/g | 4.83 | mg/g |
| (HPLC-UV) | | | |
| Impurities | All individual impurities >0.1% area to be reported. | | |
| (HPLC-UV) | RRT 0.44-0.46 ≤1.5% area | | |
| | No other single impurities >0.5% area | | |
| RRT 0.40 | ≤0.5% area | 0.15% | |
| RRT 0.46 | ≤1.5% area | 0.21% | |
| RRT 1.49 | ≤0.5% area | 0.24% | |
| RRT 2.59 | ≤0.5% area | 0.12% | |
| Total sum of | ≤2.0% area | 0.7% | |
| impurities | | | |
| Total microbial | Bacteria ≤10² cfu/g | <10 | cfu/g |
| count | Fungi ≤10¹ cfu/g | <10 | cfu/g |

| | | |
|---|---|---|
| Absence of | Absent | Absent |
| P. aeruginosa | | |
| Absence of | Absent | Absent |
| S. aureus | | |

TABLE 45
CLINICAL PHASE 2B BATCHES FROM PHASE 2 STUDY CT327-2003

| Test | Specification | PD11189 | | PD11296 | | PD11299 | |
|---|---|---|---|---|---|---|---|
| Appearance | Homogeneous white | Homogeneous off- | | Homogeneous | | Homogeneous off- | |
| | to off-white | white ointment | | white ointment | | white ointment | |
| Identity | Conforms to reference | Conforms to | | Conforms to | | Conforms to | |
| (HPLC-UV) | material (RT ± 10%) | reference material | | reference material | | reference material | |
| Preservative | Presence of | Present | | Present | | Present | |
| | Benzyl Alcohol | | | | | | |
| Viscosity | To be reported | 1200000 | cP | 1400000 | cP | 1720000 | cP |
| Extractable weight | 20.0 g to 20.2 g | 20.0 | g | NT* | | NT* | |
| Density | To be reported | 0.893 | g/cm³ | 0.851 | g/cm³ | 0.856 | g/cm³ |
| Benzyl Alcohol | 4.5% to 5.5% w/w | 4.9% | w/w | 4.8% | w/w | 5.0% | w/w |
| Assay (HPLC-UV) | | | | | | | |
| CT327 Assay | 4.50 mg/g-5.50 mg/g | 4.83 | mg/g | 4.77 | mg/g | 4.98 | mg/g |
| (HPLC-UV) | | | | | | | |
| Related | All individual impurities ≥0.1% area to be reported. | | | | | | |
| Substances | RRT 0.44-0.47 ≤1.0% (release limit) | | | | | | |
| (HPLC-UV) | No other single related substances ≥0.5% | | | | | | |
| RRT 0.40-0.47 | ≤1.0% | 0.15% | | 0.3% | | 0.3% | |
| Total sum of Related | ≤2.0% area | 0.1% | | 0.7% | | 0.6% | |
| Substances | | | | | | | |
| Total microbial | Bacteria ≤10² cfu/g | <10 | cfu/g | <10 | cfu/g | <10 | cfu/g |
| count | Fungi ≤10¹ cfu/g | <10 | cfu/g | <10 | cfu/g | <10 | cfu/g |

| | | | | |
|---|---|---|---|---|
| Absence of | Absent in 1 g | Absent | Absent | Absent |
| P. aeruginosa | | | | |
| Absence of | Absent in 1 g | Absent | Absent | Absent |
| S. aureus | | | | |

NT: Not Tested
*The Extractable Weight test was deleted from the specification by the time of manufacture of these batches, as it was considered not a critical test at this stage in development.

TABLE 46
SUMMARY OF ACCEPTANCE CRITERIA

| Test | Specification | PD13063 | | PD13064 | | PD13065 | |
|---|---|---|---|---|---|---|---|
| Appearance | Homogeneous white to | Homogeneous off- | | Homogeneous | | Homogeneous | |
| | off-white ointment | white ointment | | white ointment | | white ointment | |
| Identity | Conforms to reference | Conforms to | | Conforms to | | Conforms to | |
| (HPLC-UV) | material (RT ± 10%) | reference material | | reference material | | reference material | |
| Preservative | Presence of | Present | | Present | | Present | |
| | Benzyl Alcohol | | | | | | |
| Viscosity | To be reported | 1,400,000 | cP | 960,000 | cP | 1,100,000 | cP |
| Density | To be reported | 0.892 | g/cm³ | 0.893 | g/cm³ | 0.895 | g/cm³ |
| Benzyl Alcohol | 4.5% to 5.5% w/w | 4.7% | w/w | 5.0% | w/w | 5.0% | w/w |
| Assay (HPLC-UV) | | | | | | | |
| CT327 Assay | 4.50 mg/g-5.50 mg/g | 4.69 | mg/g | 5.02 | mg/g | 4.86 | mg/g |
| (HPLC-UV) | | | | | | | |
| Related Substances | All single related | 0.2% area (RRT 1.80) | | 0.2% area (RRT 1.79) | | 0.2% area (RRT 1.80) | |
| (HPLC-UV) | substances ≤0.5% area | | | | | | |
| RRT 0.40-0.47 | ≤1.0% area | 0.2% area (RRT 0.45) | | 0.2% area (RRT 0.45) | | 0.2% area (RRT 0.45) | |
| Total sum of Related | ≤2.0% area | 0.3% | area | 0.4% | area | 0.3% | area |
| Substances | | | | | | | |
| Total microbial | TAMC ≤10² cfu/g | <10 | cfu/g | <10 | cfu/g | <10 | cfu/g |
| count | TYMC ≤10¹ cfu/g | <10 | cfu/g | <10 | cfu/g | <10 | cfu/g |

| | | | | |
|---|---|---|---|---|
| Absence of | Absent in 1 g | Absent | Absent | Absent |
| P. aeruginosa | | | | |
| Absence of | Absent in 1 g | Absent | Absent | Absent |
| S. aureus | | | | |

NT: Not Tested

### Example 6: SNA-120 Ointment Stability Studies

### Summary

Formal ICH stability studies have been performed with previous cGMP clinical batches of SNA-120 ointment 0.5% w/w. The stability data are summarized below in Tables 47-50, and indicate a single main degradation pathway in the SNA-120 active ointments, i.e., hydrolysis to the amide RRT 0.40-0.47 (FIG. 4). The amide levels for the ointments have remained within the specification limits for the duration of the proposed shelf-life. All other measured product attributes do not show any significant differences for all time points and conditions tested during the stability studies. The stability test methods have been successfully validated, and the validation protocols and validation reports are presented above.

TABLE 47
SUMMARY OF STABILITY RESULTS ON SNA-120
0.5% W/W OINTMENT BATCH PD11189 AT 5° C.

| | | | | Related substances by | | Total | | |
|---|---|---|---|---|---|---|---|---|
| | | | | RRT (% Area) | | Sum of | | |
| | | Benzyl | | | No other | Related | | |
| | | Alcohol | | | single | Substances | | |
| Batch | | Assay | Assay | RRT | related | (% | Viscosity | Density |
| PD11189 | Appearance | (% w/w) | (mg/g) | 0.40-0.47 | substance | Area) | (cP) | (g/cm³) |
| Specification | Type III Amber glass | 4.4-5.5 | 4.25-5.75 | ≤3.0 | ≥0.5 | ≤4.0 | To be | To be |
| | jar, black urea/PVDC | | | | | | reported | reported |
| | screw cap lid. | | | | | | | |
| | Homogeneous white to | | | | | | | |
| | off-white ointment | | | | | | | |
| Initial | Type III Amber glass | 4.9 | 4.68 | 0.15 | <0.1 | 0.1 | 1200000 | 0.893 |
| | jar, black urea/PVDC | | | | | | | |
| | screw cap lid. | | | | | | | |
| | Homogeneous off- | | | | | | | |
| | white ointment | | | | | | | |

5° C.

| | | | | | | | | |
|---|---|---|---|---|---|---|---|---|
| 1 month | Type III Amber glass | | NT | NT | NT | NT | NT | NT |
| | jar, black urea/PVDC | | | | | | | |
| | screw cap lid. | | | | | | | |
| | Homogeneous off- | | | | | | | |
| | white ointment | | | | | | | |
| 2 months | Type III Amber glass | NT | NT | NT | NT | NT | NT | NT |
| | jar, black urea/PVDC | | | | | | | |
| | screw cap lid. | | | | | | | |
| | Homogeneous off- | | | | | | | |
| | white ointment | | | | | | | |
| 3 months | Type III Amber glass | NT | NT | NT | NT | NT | NT | NT |
| | jar, black urea/PVDC | | | | | | | |
| | screw cap lid. | | | | | | | |
| | Homogeneous off- | | | | | | | |
| | white ointment | | | | | | | |

NT = Not Tested
*The Stability Protocol only mandates testing of the 5° C. stored sample when there is an OOS observation for a sample stored at one of the higher temperature conditions.

TABLE 48
SUMMARY OF STABILITY RESULTS ON SNA-120 0.5% W/W OINTMENT BATCH PD11189 AT
25° C./60% RH

| | | | | Related substances | | Total | | |
|---|---|---|---|---|---|---|---|---|
| | | | | by RRT | | Sum of | | |
| | | | | (% Area) | | Related | | |
| | | Benzyl | | | No other single | Substances | | |
| Batch | | Alcohol | | RRT | related | (% | Viscosity | Density |
| PD11189 | Appearance | Assay (% w/w) | | 0.40-0.47 | substance | Area) | (cP) | (g/cm³) |
| | | | Assay | | | | | |
| | | | (mg/g) | | | | | |
| Specification | Type III Amber glass | 4.4-5.5 | 4.25-5.75 | ≤3.0 | ≥0.5 | ≤4.0 | To be | To be |
| | jar, black urea/PVDC | | | | | | reported | reported |
| | screw cap lid. | | | | | | | |
| | Homogeneous white to | | | | | | | |
| | off-white ointment | | | | | | | |
| Initial | Type III Amber glass | 4.9 | 4.68 | 0.15 | <0.1 | 0.1 | 1200000 | 0.893 |
| | jar, black urea/PVDC | | | | | | | |
| | screw cap lid. | | | | | | | |
| | Homogeneous off- | | | | | | | |
| | white ointment | | | | | | | |

25° C./60% RH

| | | | | | | | | |
|---|---|---|---|---|---|---|---|---|
| 1 month | Type III Amber glass | 5.1 | 4.61 | 0.15 | <0.1 | 0.2 | NT | NT |
| | jar. | | | | | | | |
| | black urea/PVDC | | | | | | | |
| | screw cap | | | | | | | |
| | lid. Homogeneous off- | | | | | | | |
| | white | | | | | | | |
| | ointment | | | | | | | |
| 2 months | Type III Amber glass | 5.I | 4.83 | 0.15 | <0.1 | 0.1 | NT | NT |
| | jar. | | | | | | | |
| | black urea/PVDC | | | | | | | |
| | screw cap | | | | | | | |
| | lid. Homogeneous off- | | | | | | | |
| | white | | | | | | | |
| | ointment | | | | | | | |
| 3 months | Type 1T1 Amber glass | 5.1 | 4.84 | 0.17 | <0.1 | 0.2 | NT | NT |
| | jar. | | | | | | | |
| | black urea/PVDC | | | | | | | |
| | screw cap | | | | | | | |
| | lid. Homogeneous off- | | | | | | | |
| | white | | | | | | | |
| | ointment | | | | | | | |
| 6 months | Type III Amber glass | 4.9 | 4.81 | 0.20 | <0.1 | 0.2 | NT | NT |
| | jar. | | | | | | | |
| | black urea/PVDC | | | | | | | |
| | screw cap | | | | | | | |
| | lid. Homogeneous off- | | | | | | | |
| | white | | | | | | | |
| | ointment | | | | | | | |
| 9 months | Type III Amber glass | 5.0 | 4.83 | 0.21 | <0.1 | 0.2 | NT | NT |
| | jar. | | | | | | | |
| | black urea/PVDC | | | | | | | |
| | screw cap | | | | | | | |
| | lid. Homogeneous of | | | | | | | |
| | ointment | | | | | | | |
| 12 months | Type 1T1 Amber glass | 5.0 | 4.85 | 0.25 | <0.1 | 0.3 | 1470000 | NT |
| | jar. | | | | | | | |
| | black urea/PVDC | | | | | | | |
| | screw cap | | | | | | | |
| | lid. Homogeneous off- | | | | | | | |
| | white | | | | | | | |
| | ointment | | | | | | | |
| | | | Assay | | | | | |
| | | | (mglg) | | | | | |
| Specification | Type III Amber glass | 4.5-5.5 | 4.25-5.75 | <3.0 | >0.5 | ≤4.0 | To be | To be |
| | jar. black urea/PVDC | | | | | | reported | reported |
| | screw cap lid. | | | | | | | |
| | Homogeneous white to | | | | | | | |
| | off-white ointment | | | | | | | |
| Initial | Type III Amber glass | 4.9 | 4.68 | 0.15 | <0.1 | 0.1 | 1200000 | 0.893 |
| | jar. black urea/PVDC | | | | | | | |
| | screw cap lid. | | | | | | | |
| | Homogeneous off- | | | | | | | |
| | white ointment | | | | | | | |

25° C./60% RH

| | | | | | | | | |
|---|---|---|---|---|---|---|---|---|
| 18 months | Type III Amber glass | 4.9 | 4.80 | 0.46 | <0.1 | 0.5 | NT | NT |
| | jar. black urea/PVDC | | | | | | | |
| | screw cap lid. | | | | | | | |
| | Homogeneous off- | | | | | | | |
| | white ointment | | | | | | | |
| 24 months | Type III Amber glass | 4.9 | 4.85 | 0.43 | 0.23 (RRT 3.57) | 0.7 | 1,450,000 | NT |
| | jar. black urea/PVDC | | | | | | | |
| | screw cap lid. | | | | | | | |
| | Homogeneous off- | | | | | | | |
| | white ointment | | | | | | | |
| 36 months | Type III Anther glass | 5.0 | 4.76 | 0.85 | <0.1 | 0.9 | 1,200.000 | NT |
| | jar. black urea/PVDC | | | | | | | |
| | screw cap lid. | | | | | | | |
| | Homogeneous off- | | | | | | | |
| | white ointment | | | | | | | |
| 48 months | Type III Amber glass | 4.9 | 4.73 | 1.44 | <0.1 | 1.4 | 2,000,000 | NT |
| | jar. black urea/PVDC | | | | | | | |
| | screw cap lid. | | | | | | | |
| | Homogeneous off- | | | | | | | |
| | white ointment | | | | | | | |
| 60 months | Type III Amber glass | 4.9 | 4.73 | 2.59 | 0.10 (RRT 0.71) | 2.7 | NT | NT |
| | jar. black urea/PVDC | | | | | | | |
| | screw cap lid. | | | | | | | |
| | Homogeneous off- | | | | | | | |
| | white ointment | | | | | | | |
| 68 months | Type III Amber glass | 4.7 | 4.77 | 2.00 | 0.17 (RRT 0.71) | 2.3 | NT | NT |
| | jar. black urea/PVDC | | | | 0.10 (RRT 0.84) | | | |
| | screw cap lid. | | | | | | | |
| | Homogeneous off- | | | | | | | |
| | white ointment | | | | | | | |

NT = Not Tested

TABLE 49
SUMMARY OF STABILITY RESULTS ON SNA-120 0.5% W/W OINTMENT BATCH PD11189 AT
40° C./75% RH

| | | Benzyl | | Related Substances by RRT | | Total | | |
|---|---|---|---|---|---|---|---|---|
| | | Alcohol | | (% Area) | | Sum of | | |
| | | Assay | Assay | | No other single | Related | Viscosity | Density |
| Batch PD11189 | Appearance | (% w/w) | (mg/g) | RRT 0.40-0.47 | related substance | Substances (% | (cP) | (g/cm³) |
| Specification | Type III Amber glass | 4.5-5.5 | 4.25-5.75 | ≤3.0 | ≥0.5 | ≥4.0 | To be | To be |
| | jar. | | | | | | reported | reported |
| | black urea/PVDC | | | | | | | |
| | screw cap | | | | | | | |
| | lid. Homogeneous | | | | | | | |
| | white to | | | | | | | |
| | off-white ointment | | | | | | | |
| Initial | Type III Amber glass | 4.9 | 4.68 | 0.15 | <0.1 | 0.1 | 1200000 | 0.893 |
| | jar. | | | | | | | |
| | black urea/PVDC | | | | | | | |
| | screw cap | | | | | | | |
| | lid. Homogeneous off- | | | | | | | |
| | white | | | | | | | |
| | ointment | | | | | | | |

40° C./75% RH

| | | | | | | | | |
|---|---|---|---|---|---|---|---|---|
| 1 month | Type III Amber glass | 5.3 | 5.22 | 0.24 | <0.1 | 0.2 | NT | NT |
| | jar. | | | | | | | |
| | black urea/PVDC | | | | | | | |
| | screw cap | | | | | | | |
| | lid. Homogeneous off- | | | | | | | |
| | white | | | | | | | |
| | ointment | | | | | | | |
| 2 months | Type DI Amber glass | 5′ | 5./1 | 0.39 | <0.1 | 0.4 | NT | NT |
| | jar. | | | | | | | |
| | black urea/PVDC | | | | | | | |
| | screw cap | | | | | | | |
| | lid. Homogeneous off- | | | | | | | |
| | white | | | | | | | |
| | ointment | | | | | | | |
| 3 months | Type III Amber glass | 5.1 | 4.93 | 0.62 | <0.1 | 0.6 | NT | NT |
| | jar. | | | | | | | |
| | black urea/PVDC | | | | | | | |
| | screw cap | | | | | | | |
| | lid. Homogeneous off- | | | | | | | |
| | white | | | | | | | |
| | ointment | | | | | | | |
| 6 months | Type III Amber glass | 4.9 | 4.86 | 1.7 | <0.1 | 1.7 | 2030000 | NT |
| | jar. | | | | | | | |
| | black urea/PVDC | | | | | | | |
| | screw cap | | | | | | | |
| | lid. Homogeneous off- | | | | | | | |
| | white | | | | | | | |
| | ointment | | | | | | | |

NT = Not Tested

TABLE 50
SUMMARY OF MICROBIOLOGICAL RESULTS FOR SNA-120 0.5% W/W
OINTMENT BATCH PD11189 AT 40° C./75% RH (6 MONTHS)
AND AT 25° C./60% RH (60 MONTHS)

| | Total Aerobic | Total Combined | Absence of | Absence of |
|---|---|---|---|---|
| Batch | Microbial Count | Yeasts and | Pseudomonas | Staphylococcus |
| PD11189 | (TAMC) | Moulds (TYMC) | aeruginosa | aureus |
| Specification | ≤10² cfu/g | ≤10¹ cfu/g | Absent in 1 g | Absent in 1 g |
| | (max count = | (max count = | | |
| | 200 cfu/g) | 200 cfu/g) | | |
| Initial | <10 cfu/g | <10 cfu/g | Absent in 1 g | Absent in 1 g |

40° C. 175% RH

| | | | | |
|---|---|---|---|---|
| 6 months | 10 cfu/g | <10 cfu/g | Absent in 1 g | Absent in 1 g |

25° C./60% RH

| | | | | |
|---|---|---|---|---|
| 12 months | <10 cfu/g | <10 cfu/g | Absent in 1 g | Absent in 1 g |
| 24 months | <10 cfu/g | <10 cfu/g | Absent in 1 g | Absent in 1 g |
| 36 months | <10 cfu/g | <10 cfu/g | Absent in 1 g | Absent in 1 g |
| 48 months | <10 cfu/g | <10 cfu/g | Absent in 1 g | Absent in 1 g |
| 60 months | <10 cfu/g | <10 cfu/g | Absent in 1 g | Absent in 1 g |

### Conclusions

On the basis of the stability data to date and ICH Guidance (Q1E), a drug product shelf-life of 68 months at ambient temperature has been assigned to the 0.5% w/w SNA-120 ointment.

### Example 7: SNA-120 Ointment Formulation Manufacturing Instructions

This example provides the instructions for manufacturing a 40 kg batch of 0.5% and 0.05% SNA-120 ointment formulations.

### 1—Composition

Tables 51 and 52 provide the composition of the 0.5% and 0.05% SNA-120 ointment formulations, respectively, prepared according to manufacturing instructions.

TABLE 51
COMPOSITION OF SNA-120 (0.5%)
OINTMENT FORMULATION

| | | |
|---|---|---|
| Paraffin White Soft | 75.00 | g |
| White beeswax | 8.00 | g |
| Stearyl alcohol | 3.00 | g |
| Cholesterol | 3.00 | g |
| Benzyl alcohol | 5.00 | g |
| Butylhydroxytoluene | 0.10 | g |
| SNA-120 | 0.5 | g |
| Propylene glycol | 5.4 | g |
| Total | 100 | g |

TABLE 52
COMPOSITION OF SNA-120 (0.05%)
OINTMENT FORMULATION

| | | |
|---|---|---|
| Paraffin White Soft | 75.00 | g |
| White beeswax | 8.00 | g |
| Stearyl alcohol | 3.00 | g |
| Cholesterol | 3.00 | g |
| Benzyl alcohol | 5.00 | g |
| Butylhydroxytoluene | 0.10 | g |
| SNA-120 | 0.05 | g |
| Propylene glycol | 5.85 | g |
| Total | 100 | g |

### 2—Equipment

A Becomix RW-50 homogenizing mixer and stainless steel container are employed.

### 3—Dispensing

Actual weight, batch number and in-house expiration date are documented as required by the weighing record.

In-process control (IPC) 1: The identity and the actual weight of each weighed raw material conforms to the requirements.

### 4—Calculation of Weights

The quantities of the API SNA-120 to be used for this batch have to be calculated by applying the calculation formula as shown hereafter and will be documented in the weighing record (4.3 below) and at step 6.3 below.

4.1—if One Lots of API SNA-120 is Used

The assay value of the lot of API SNA-120 is determined (assay C₁). If the SNA-120 assay C₁ is above 100.0%, then calculations are performed with C₁=100.0%.

M_{w}=quantity corrected by factor [kg]

For the 0.5% SNA-120 formulation, Mₙ₁=(0.2000 kg)×(100%/assay=kg of SNA-120 weighed.

For the 0.05% SNA-120 formulation, Mₙ₁=(0.0200 kg)×(100%/assay C₁)=kg of SNA-120 weighed.

4.2—if Two Lots of API SNA-120 are Used

M_{#2}=Quantity of the second API batch to be used [kg]

M_{#1}=Available quantity of the first API batch [kg]

Mₜ=Theoretical quantity=0.2000 kg for 0.5% SNA-120 formulation & and 0.0200 kg for 0.5% SNA-120.

C₁, C₂=Assay value of API SNA-120 batches 1 and 2, respectively.

M_{N1}, M_{N2}=Quantity corrected by factor of API SNA-120 batches 1 and 2, respectively.

If the SNA-120 assay C is above 100.0%, then calculations are performed with C=100.0%.

M_{N1}=((M_{#1} kg)×(C₁%))/(100%)

For the 0.5% SNA-120 formulation, M#₂=0.2000 kg−M_{N1} kg=[kg]

For the 0.05% SNA-120 formulation, M_{#2}=0.0200 kg−M_{N1} kg=[kg]

M_{N2}=((M_{#2} kg)×(100%))/(C₂%)=[kg]

4.3—Weighing Record

Target weights of the components of the 0.5% and 0.05% SNA-120 Ointment Formulations for a batch size of 40 kg to be weighed are depicted in Tables 53 and 54, respectively. Note that SNA-120 has to be removed from −20° C. storage at least 4 hours before dispensing.

TABLE 53
TARGET WEIGHTS OF SNA-120 (0.5%)
OINTMENT FORMULATION COMPONENTS
FOR BATCH SIZE OF 40 KG

| Raw Material | Target Weight |
|---|---|
| Paraffin White Soft | 30.000 kg |
| White beeswax | 3.200 kg |
| Stearyl alcohol | 1.200 kg |
| Cholesterol | 1.200 kg |
| Benzyl alcohol | 2.000 kg |
| Butylhydroxytoluene | 0.040 kg |
| SNA-120 | * |
| Propylene glycol | 1.440 kg |
| Propylene glycol | 0.720 kg |

* Weight of SNA-120 batch(es) as determined in steps 4.1 & 4.2.

TABLE 54
TARGET WEIGHTS OF SNA-120 (0.05%)
OINTMENT FORMULATION COMPONENTS
FOR BATCH SIZE OF 40 KG

| Raw Material | Target Weight |
|---|---|
| Paraffin White Soft | 30.000 kg |
| White beeswax | 3.200 kg |
| Stearyl alcohol | 1.200 kg |
| Cholesterol | 1.200 kg |
| Benzyl alcohol | 2.000 kg |
| Butylhydroxytoluene | 0.040 kg |
| SNA-120 | * |
| Propylene glycol | 1.560 kg |
| Propylene glycol | 0.780 kg |

* Weight of SNA-120 batch(es) as determined in steps 4.1 & 4.2.

### 5—Preparation of Phase I

5.1

30.000 kg of Paraffin white soft, 3.200 kg of White Beeswax, 1.200 kg of Stearyl alcohol, and 1.200 kg of Cholesterol are given into a stainless steel container and heated up 70° C. (65-75° C.). The mixture is stirred and homogenized (circulation closed) until a homogeneous phase is formed. The homogenizing speed is 25-35 Hz and the stirring speed is 3-4 m/s. If necessary, the Becomix can be opened to scrape off solid residue.

5.2

IPC 2: The mixture is homogeneous and all components are melted completely.

### 6—Preparation of Phase II

6.1

2.000 kg of Benzyl alcohol and 0.040 kg of Butylhydroxytoluene are given into a stainless steel pot while stirring using a propeller stirrer and dissolved. The Stirring time is at least 60 minutes, until IPC 3 complies. The stirring speed is 140-170 rpm.

6.2

IPC 3: The solution is homogeneous, clear and free of undissolved particles.

6.3

The measured quantity of SNA (determined above) was allowed to thaw at room temperature for approx. 4 hours and are dissolved in the solution of 6.1 while stirring. The stirring speed is 140-170 rpm and the stirring time is at least 30 minutes, until IPC 4 complies.

6.4

IPC 4: The solution is homogeneous, clear and free of undissolved particles.

6.5

1.440 kg of propylene glycol (for the 0.5% SNA-120 formulation) or 1.560 kg of propylene glycol (for the 0.05% SNA-120 formulation) is dissolved in the solution of 6.3 while stirring at a speed of 140-170 rpm. The stirring time is at least 10 minutes, until IPC 5 complies.

6.6

IPC 5: The solution is homogeneous, clear and free of undissolved particles.

### 7—Combining of Phase I and Phase II

7.1

The mixture 5.2 is slowly cooled down to 40° C. (35-45° C.) while stirring. The stirring speed is 4-5 m/s. If needed, cooling can be switched on.

7.2

The solution 6.5 is slowly given to the mass 7.1 in the Becomix. The addition is performed carefully and steadily while avoiding adding large aliquots too quickly. If needed, cooling can be switched on. The agitation speed is 4-5 m/s.

7.3

0.720 kg of propylene glycol (for the 0.5% SNA-120 formulation) or 0.780 kg of propylene glycol (for the 0.05% SNA-120 formulation) is given into the emptied steel pot. Stir (140-170 rpm) for at least 1 minute to ensure the stainless steel pot is fully rinsed.

7.4

The resulting rinse is slowly given into the Becomix.

7.5

After adding the propylene glycol the mass is homogenized under circulation:

Stirring speed: 5.5-6.5 m/s

Temperature: 40° C. (35-45° C.)

Homogenizer setting: 35-45 Hz

Homogenizer: Circulation open

Homogenizer time: 25 min

Stirring time: at least 25 minutes, until IPC 6 complies.

If needed, cooling can be switched on.

7.6

IPC 6: The mixture is homogenous and free of lumps.

7.7

While stirring, but without using the homogenizer, the mixture is cooled to below 30° C. The agitation speed is 5.5-6.5 m/s. If needed, cooling can be switched on. The temperature is 28-30° C.

7.8

Once the mixture has cooled to below 30° C., the homogenizer is turned on for 10 s. The homogenizer is set at 35-45 Hz and the agitation speed is 5.5-6.5 m/s. If needed, cooling can be switched on.

7.9

IPC 7: The ointment is homogeneous and shows no separation of phases.

### 8—Discharge in Stainless Steel Container

8.1

The preparation is transferred into a stainless steel container via homogenizer. The homogenizer is set at 25-30 Hz and the agitation speed is 5.5-6.5 m/s.

Samples for homogeneity testing are taken from the following positions and labelled accordingly: ‘Beginning of discharging’, ‘Middle of discharging’, and ‘End of discharging’. The stainless steel container is closed, sealed and labeled with material name, material and batch number and stored at ambient conditions whilst awaiting homogeneity results. The first surge of material is discarded before the first sample is taken.

8.2

IPC 8: The assay of SNA-120 is 95-105%.

### 9—Determination of Yield

The output weight of the batch in [kg] is measured.

Yield [%]=((output weight [kg])/(40 kg batch size))×100%=[%]

### Example 8: A Randomized, Placebo-Controlled Phase I Study to Evaluate the Safety and Tolerability of 0.5% W/W CT327 when Applied Twice Daily to Stable Psoriasis Vulgaris Patients' Skin Lesions

### Introduction

Psoriasis is defined as a chronic inflammatory skin disease affecting between 1% and 2% of the world population and classically characterized by thickened, red areas of skin covered with silvery scales. The extent of the skin involvement can range from discrete, localized areas to generalized body involvement. Joints, nails and mucous membranes may also be affected by this disease.

Keratinocytes in the basal layer of the epidermis secrete nerve growth factor (NGF). NGF released from keratinocytes acts in an autocrine manner stimulating the hyperproliferation of normal keratinocytes. Endogenous NGF also protects human keratinocytes from apoptosis. These findings suggest that NGF is involved in some pathological cutaneous conditions characterized by keratinocyte hyperproliferation, inflammation and a reduced rate of apoptosis, for example psoriasis and dermatitis.

TrkA is an NGF receptor expressed by keratinocytes. CT327 has been demonstrated to be an inhibitor of TrkA kinase at nanomolar levels and has shown clear inhibitory activity of human keratinocyte proliferation in vitro. CT327 is poorly systemically absorbed, therefore restricting any activity by CT327 to the keratinocyte layer and thereby reducing the risk of systemic side effects. Systemic and topical toxicological studies and clinical experience have indicated that CT327 is safe and well tolerated.

Topical treatment continues to be the mainstay in the management of psoriasis. Many patients continue to use their preferred topical treatment even as the disease progresses in severity, with systemic drugs added to the treatment regime. The majority of physicians prescribe topical drugs only or a combination of topical and systemic drug classes, with the use of combinations increasing with severity.

CT327 is proposed as a new topical treatment for psoriasis, in addition and/or as an alternative to the currently available topical drugs such as: corticosteroids, coal tar preparations, dithranol (1,8 dihydroxy 9 anthrone), vitamin D analogues (calcipotriol, calcitriol, tacalcitol), retinoids (tazarotene) and immunosuppressive drugs (calcineurin inhibitors).

CT327 amide RRT 0.40-0.47 is a potential degradation product of the drug substance CT327, which is an organic amide impurity which was observed at a relative retention time of 0.44-0.46 in the ointment formulation.

### Study Objectives

The primary objective of this study was to evaluate the safety and tolerability of CT327 when administered twice daily (bid) as a topical ointment to lesions of psoriasis vulgaris in comparison to placebo.

The secondary objectives of this study were: 1) to determine the pharmacokinetic (PK) profile of CT327 in plasma and urine following bid administration of CT327 as a topical ointment to lesions of psoriasis vulgaris; 2) to determine the PK profile of CT327 amide RRT 0.40-0.47 (a potential degradant of CT327) in plasma and urine following bid administration of CT327 as a topical ointment to lesions of psoriasis vulgaris; 3) to assess the efficacy of CT327 when administered bid as a topical ointment to lesions of psoriasis vulgaris in comparison to placebo; and 4) to evaluate the application site tolerability of CT327 when administered bid as a topical ointment to lesions of psoriasis vulgaris in comparison to placebo.

### Investigational Plan

Overall Study Design and Plan

This was a Phase I, single-center, randomized, double-blind, placebo-controlled study in male and female patients with stable psoriasis vulgaris aged >18 and <71 years. Sixteen patients were enrolled in 2 treatment groups. Twelve patients received bid applications of 0.5% w/w CT327 from Days 1 to 6 and a single application on Day 7, to a total area of 50 cm² (±10 cm²) which could be made up of individual plaques which were >10 cm². Four patients received bid applications of matched placebo of CT327 from Days 1 to 6 and a single application on Day 7, to a total area of 50 cm² (±10 cm²) which could be made up of individual plaques which were ≥10 cm². Patients attended a screening visit within 21 days prior to Day 1. Patients were then admitted to the CPU in the afternoon of Day −1 and remained resident in the CPU until the morning of Day 8. Patients attended a follow-up visit 7 to 10 days after the last treatment administration on Day 7.

Treatments

Patients received applications of 0.5% w/w CT327 ointment or matching placebo ointment across the selected target lesion(s) bid at 0 hours and 12 hours on Days 1 to 6 and once at 0 hours on Day 7. The total application did not exceed approximately 1 g of 0.5% w/w CT327 or matching placebo at each dosing time, across all lesion(s). The ointment was applied by the CPU staff, using a spatula and finger cot.

The identity of investigational products were CT327 (batch number PD11034) and placebo (batch number PD11031).

CT327 Amide RRT 0.40-0.47 was present (end of shelf-life specification limit <6.0%) in the cream used in previous clinical studies, consequently it was decided to monitor it as a potential CT327 degradant in the current ointment formulation. Preliminary laboratory analyses confirmed that CT327 amide levels in the new ointment formulation are much lower compared to the cream (0.21% at clinical batch release). Therefore, a tighter specification end of shelf-life limit (<1.5%) for this degradant has been proposed. The cream formulation demonstrated good safety and tolerability profile, including no application site irritation.

Pharmacokinetic, Safety and Efficacy Variables

A summary table of the assessments performed in the study is given in Table 55.

TABLE 55
OVERVIEW OF STUDY ASSESSMENTS

| | Screening | | Study Day | | | | | | Follow-up |
|---|---|---|---|---|---|---|---|---|---|
| | Day −21 to | Admission | | | | Days 4 | | | 7 to 10 days |
| Study Procedure | Day −2 | Day −1 | Day 1 | Day 2 | Day 3 | to 6 | Day 7 | Day 8 | post-last dose |
| Resident in the CPU | | X | X | X | X | X | X | | |
| Discharge from CPU | | | | | | | | X | |
| Informed consent | X | | | | | | | | |
| Demography and medical history | X | | | | | | | | |
| Physical examination | X | | | | | | | | X |
| 12-lead ECG | X | | X⁵ | | X⁵ | | X⁵ | | X |
| Vital signs | X | | X⁵ | | X⁵ | | X⁵ | | X |
| Clinical laboratory tests | X | | X⁵ | | X⁵ | | X⁵ | | X |
| Serum pregnancy tests¹ | X | | | | | | | | X |
| Alcohol breath test and urine | X | X | | | | | | | |
| drugs of abuse test | | | | | | | | | |
| Virology | X | | | | | | | | |
| Inclusion/exclusion criteria | X | X | X | | | | | | |
| satisfaction | | | | | | | | | |
| CT327/placebo administration² | | | X | X | X | X | X | | |
| PK blood samples³ | | | X | | X | | X | N | |
| PK urine samples⁴ | | | X | X | X | | X | | |
| Completion of PGA | X | | X | | | | X | | X |
| Application site photograph | | X | | | | | X | | |
| AL monitoring | | X | X | X | X | X | X | | X |
| Prior and concomitant | X | X | X | X | X | X | X | | X |

AE = adverse event;
CPU = clinical pharmacology unit;
ECG = electrocardiogram;
PGA = Physician's global assessment;
PK = pharmacokinetic
¹For females only
²Patients received applications of 0.5% w/w CT327 or matching placebo bid at 0 hours and 12 hours on Days 1 to 6 and once at 0 hours on Day 7.
³PK blood samples were taken at pre-dose and 0.5, 1, 2, 4 and 12 hours post-dose on Day 1, at pre-dose and 0.5, 1, 2, 4 and 12 hours post-dose on Day 3, and at pre-dose and 0.5, 1, 2, 4, 8, 12 and 24 hours post-dose on Day 7
⁴Urine was collected on Day 1 (pre-dose and 0-12 hours post-dose), Day 3 (pre-dose and 0-12 hours post-dose) and Day 7 (pre-dose and 0-12 and 12-24 hours post-dose).
⁵Was performed pre morning dose.

### Pharmacokinetic Assessments

Blood samples for PK assessments were taken at the time points displayed in Table 55.

### Safety Assessments

Safety assessments were assessed at time points displayed in Table 55.

Adverse Events

Adverse events (AEs) were elicited at the times indicated in the schedule by asking the question: “Since you were last asked, have you felt unwell or different from usual in any way?” Any adverse or unexpected events, signs and symptoms were fully recorded on the AE form including details of severity at onset, maximum severity, onset, duration, outcome and relationship to the study treatment. The type and duration of follow-up of patients after AEs was also documented. Adverse events were also reported spontaneously at any time.

Adverse events were coded to system organ class (SOC) and preferred terms (PT) using MedDRA criteria and listed, including other categorical information of interest such as onset and resolution times, time of onset and resolution relative to dose, severity at onset, maximum severity, causal relationship to study treatment, action taken and outcome.

AEs were classified as treatment-emergent (TEAE) if the AE was not present prior to administration of study treatment on Day 1 and started at or after the time of the first administration of study treatment.

Adverse events were allocated to treatment as follows: a) Pre-dose, events with onset after screening but prior to the first administration of study treatment on Day 1; b) Placebo, events with onset on or after administration of placebo bid; and c) CT327 bid, events with onset on or after administration of CT327 (0.5% w/w) bid.

Physical Examinations and Vital Signs Measurements

Each physical examination included an assessment of the following body systems: general appearance, neck and thyroid, extremities, neurological, ophthalmological, ears/nose/throat, cardiovascular, respiratory, abdominal, hepatic, musculoskeletal and dermatological.

Supine blood pressure and pulse rate were measured after 3 minutes rest. Blood pressure, pulse and oral body temperature were measured at the time points listed in Table 55.

Electrocardiograms

Two copies of a single page 12-lead ECG 10 mm/1 my, 25 mm/s with a 10 second lead II rhythm strip were recorded in triplicate at the time points listed in Table 55. ECGs were recorded following a supine rest for 5 minutes.

Clinical Laboratory Tests

The following haematology, biochemistry, and urinalysis routine tests were performed at the time points listed in Table 55.

Haematology tests: haemoglobin (Hb), mean cell haemoglobin (MCH), MCH concentration, mean cell volume, packed cell volume, platelet count, red cell count, white cell count, basophils, eosinophils, lymphocytes, monocytes, neutrophils.

Biochemistry tests: alanine transferase (ALT), alkaline phosphatase, aspartate transferase, bilirubin, calcium, chloride, creatinine, inorganic phosphorous, total cholesterol, glucose, gamma glutamyl transferase, potassium, sodium, total protein, urea, albumin, globulin.

Urinalysis tests: Blood (free Hb), glucose, ketones, bilirubin, pH, protein, urobilinogen, nitrite, specific gravity. Microscopy tests (performed in the event of abnormal urinalysis) include casts, epithelial cells, red blood cells, white blood cells.

In the event of unexplained abnormal laboratory test values, the tests were repeated and followed up until they returned to normal range or an adequate explanation for the abnormality was found.

### Efficacy Assessments

Physicians Global Assessment (PGA) scores were assessed at screening, Day 1, Day 7 and at follow-up. Digital photographs of the application sites were also taken on admission to the CPU, Day 7 and at follow-up.

### Study Populations

The pharmacokinetics population included all patients who had a fully evaluable plasma profile and had no major protocol deviations that could affect the pharmacokinetics of CT327. The safety population included all patients who had received at least one administration of study treatment. The efficacy population included all patients who were randomised and had PGA score data on Days 1 and 7. All sixteen patients completed the study per protocol.

### Pharmacokinetic Results—Determination of CT327 and CT327 Amide Levels in Human Plasma Samples by HPLC-FLD

Introduction

This project involved the determination of CT327 and CT327 amide in 240 human plasma samples derived from a study involving the evaluation of the safety and tolerability of CT327 when administered twice daily, as a topical ointment, to lesions of psoriasis vulgaris in comparison to placebo.

The analytical procedure involved extraction of CT327 and CT327 amide from human plasma by a liquid-liquid extraction. The human plasma samples were analyzed using a validated HPLC method with fluorescence detection (FLD). Quantification was achieved using CT327 and CT327 amide to internal standard peak area ratios. Concentrations of the calibration curve standards, quality control samples and study samples were determined by the method of (1/x²) weighted least squares linear regression.

The lower limits of quantification for CT327 and CT327 amide were established at 5.00 ngml⁻¹ and 2.00 ngml⁻¹, respectively. The upper limits of quantification for CT327 and CT327 amide were established at 250.10 ngml⁻¹ and 100.06 ngml⁻¹, respectively. Calibration standards, quality control samples and the incurred sample reanalysis results met the acceptance criteria, demonstrating acceptable performance of the method during the analysis of the study samples. Pharmacokinetic samples were analyzed, and surprisingly as there were no quantifiable concentrations of CT327 or CT327 amide in plasma for any of the patients in this study. This indicates that there was either no systemic exposure to CT327 or CT327 amide or that systemic exposure was too low to be quantified using the current methodology.

Materials

The analytical standards were CT327 (Serichim); CT327 amide (Serichim); and internal standard Midostaurin Hydrate (Sigma Aldrich).

For the biological matrix, blank (analyte-free) human plasma was supplied by Sera Labs, and was harvested from blood collected using K3-EDTA as the anticoagulant.

A total of 240 human plasma samples were analyzed. The samples were collected on day 1 pre-dose, 0.5, 1, 2, 4 and 12 hours; day 3 pre-dose, 0.5, 1, 2, 4 and 12 hours; and day 7 pre-dose, 0.5, 1, 2, 4, 8, 12 and 24 hours after the 1 g dose of 0.5% w/w CT327 had been administered. A further 80 samples were received from subjects who had been dosed with placebo; these were not analyzed. All samples were stored at −80° C. (±10° C.).

Methods

The extraction procedure involved a liquid-liquid extraction. A portion of the extract was injected onto the HPLC-FLD system.

For calibration curve generation, duplicate calibrators at seven different concentrations, a human plasma blank and a human plasma blank plus internal standard were prepared for each batch. The calibration curves were produced by plotting the analyte to internal standard peak area ratios (y-axis) versus the concentration. The gradient and the intercept were determined by (1/x²) weighted least squares linear regression.

The concentration of CT327 and CT327 amide in each sample was calculated from the regression equation of the standard curve from each batch as follows:

Calculated concentration of analyte=((PAR-C)/M), where: PAR=analyte to internal standard peak area ratio; C=intercept; and M=gradient.

### Sample Analysis Results

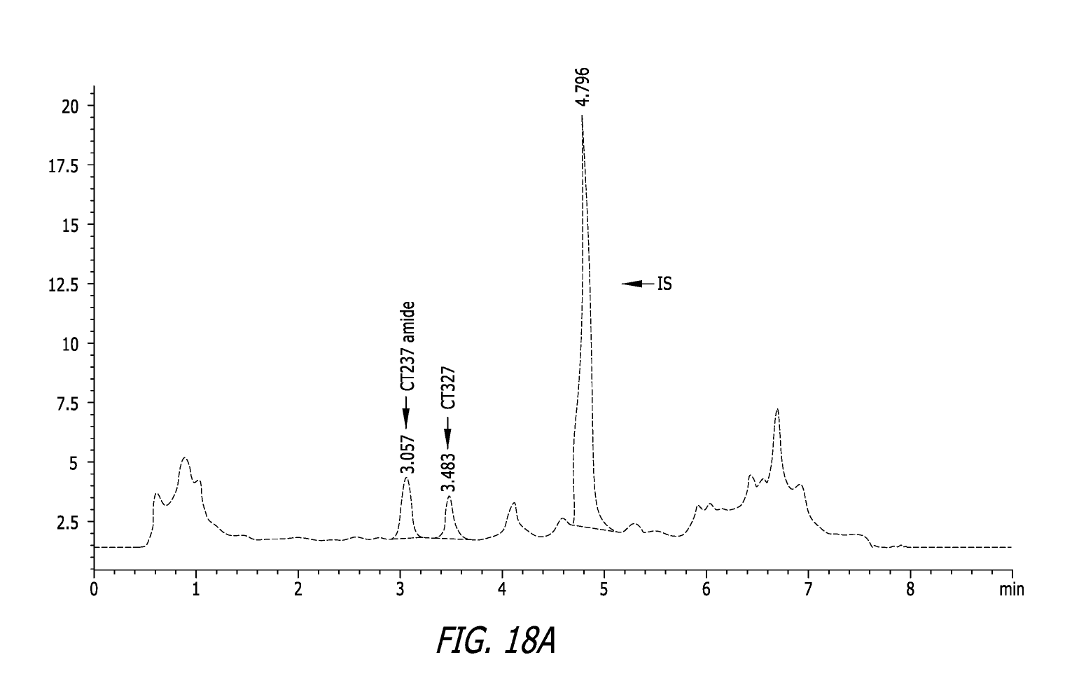

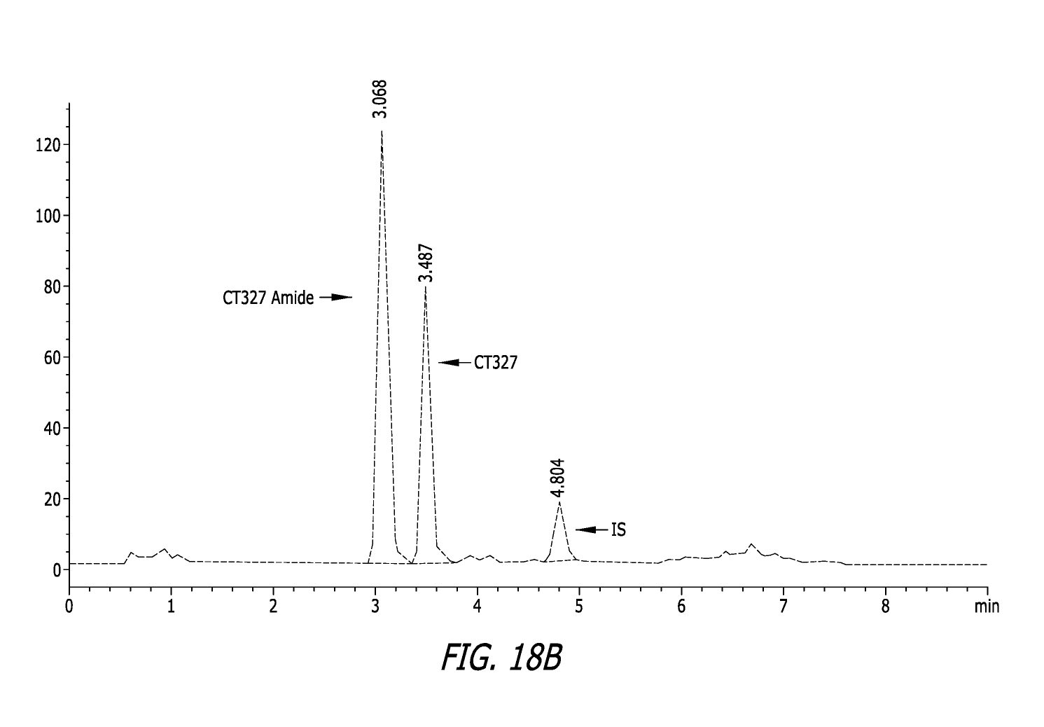

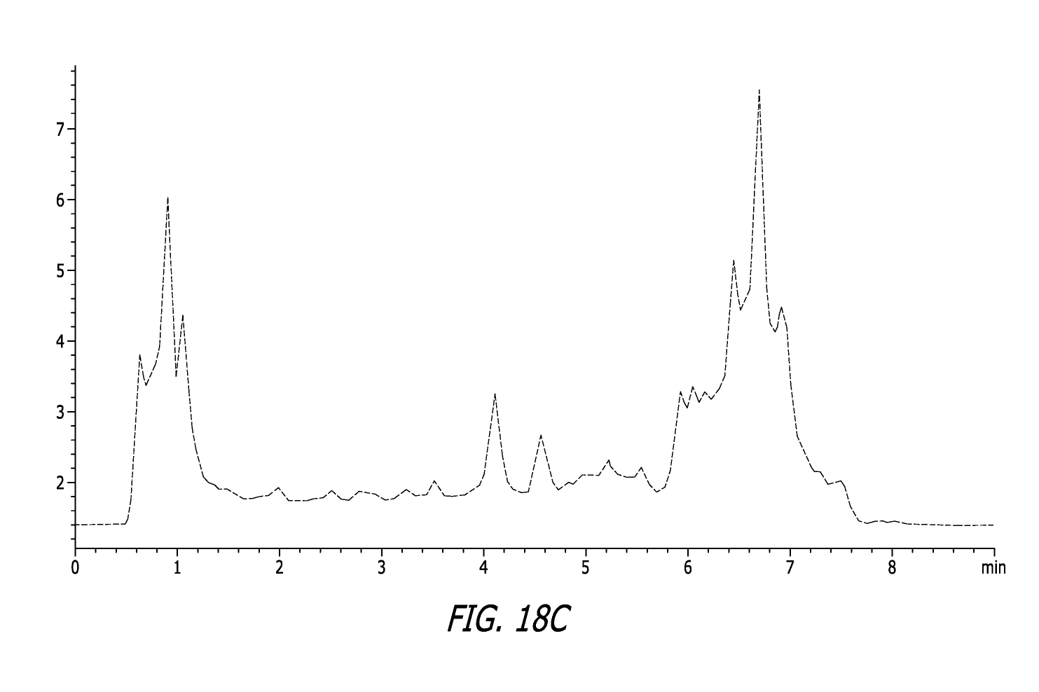

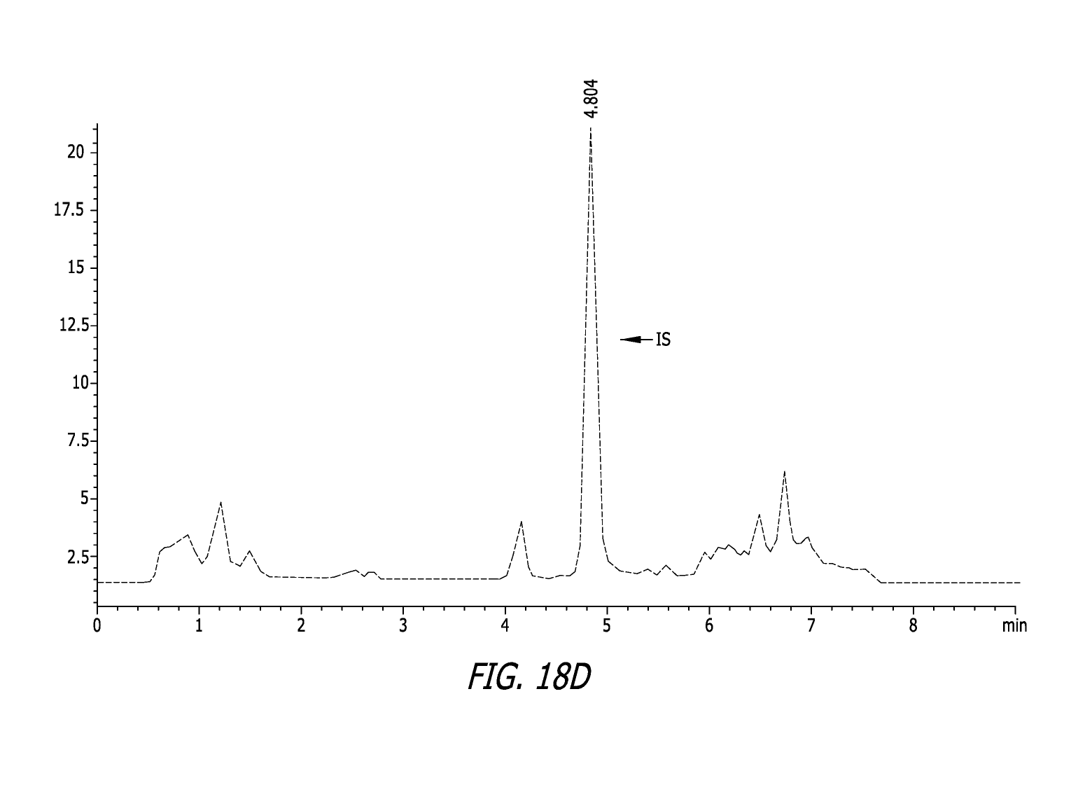

240 samples from 12 subjects (20 samples per subject) were analyzed by HPLC-FLD. The concentrations of CT327 and CT327 amide in the samples are presented in Tables 56 and 57, respectively. All samples for both analytes had concentrations below the LLOQ, and thus were reported as non-quantifiable (NQ). There were no failed batches during sample analysis. Typical chromatograms of the lower and upper limits of quantification, a blank sample and a patient plasma sample are shown in FIGS. 18A to 18D, respectively.

TABLE 56
CT327 SAMPLE ANALYSIS RESULTS

| | | CT327 Concentration (ngml⁻¹) | | | | | |
|---|---|---|---|---|---|---|---|
| | | Sub No. | | | | | |
| Day | Time | R001 | R002 | R003 | R004 | R005 | R006 |
| 1 | predose | NQ | NQ | NQ | NQ | NQ | * |
| 1 | 0.5 h | NQ | NQ | NQ | NQ | NQ | * |
| 1 | 1 h | NQ | NQ | NQ | NQ | NQ | * |
| 1 | 2 h | NQ | NQ | NQ | NQ | NQ | * |
| 1 | 4 h | NQ | NQ | NQ | NQ | NQ | * |
| 1 | 12 h | NQ | NQ | NQ | NQ | NQ | * |
| 3 | predose | NQ | NQ | NQ | NQ | NQ | * |
| 3 | 0.5 h | NQ | NQ | NQ | NQ | NQ | * |
| 3 | 1 h | NQ | NQ | NQ | NQ | NQ | * |
| 3 | 2 h | NQ | NQ | NQ | NQ | NQ | * |
| 3 | 4 h | NQ | NQ | NQ | NQ | NQ | * |
| 3 | 12 h | NQ | NQ | NQ | NQ | NQ | * |
| 7 | predose | NQ | NQ | NQ | NQ | NQ | * |
| 7 | 0.5 h | NQ | NQ | NQ | NQ | NQ | * |
| 7 | 1 h | NQ | NQ | NQ | NQ | NQ | * |
| 7 | 2 h | NQ | NQ | NQ | NQ | NQ | * |
| 7 | 4 h | NQ | NQ | NQ | NQ | NQ | * |
| 7 | 8 h | NQ | NQ | NQ | NQ | NQ | * |
| 7 | 12 h | NQ | NQ | NQ | NQ | NQ | * |
| 7 | 24 h | NQ | NQ | NQ | NQ | NQ | * |

Key
NQ Non-quantifiable
* Placebo sample, not analysed

| | | CT327 Concentration (ngml⁻¹) | | | | | |
|---|---|---|---|---|---|---|---|
| | | Sub No. | | | | | |
| Day | Time | R007 | R008 | R009 | R010 | R011 | R012 |
| 1 | predose | * | NQ | NQ | NQ | NQ | * |
| 1 | 0.5 h | * | NQ | NQ | NQ | NQ | * |
| 1 | 1 h | * | NQ | NQ | NQ | NQ | * |
| 1 | 2 h | * | NQ | NQ | NQ | NQ | * |
| 1 | 4 h | * | NQ | NQ | NQ | NQ | * |
| 1 | 12 h | * | NQ | NQ | NQ | NQ | * |
| 3 | predose | * | NQ | NQ | NQ | NQ | * |
| 3 | 0.5 h | * | NQ | NQ | NQ | NQ | * |
| 3 | 1 h | * | NQ | NQ | NQ | NQ | * |
| 3 | 2 h | * | NQ | NQ | NQ | NQ | * |
| 3 | 4 h | * | NQ | NQ | NQ | NQ | * |
| 3 | 12 h | * | NQ | NQ | NQ | NQ | * |
| 7 | predose | , | NQ | NQ | NQ | NQ | * |
| 7 | 0.5 h | * | NQ | NQ | NQ | NQ | * |
| 7 | 1 h | * | NQ | NQ | NQ | NQ | * |
| 7 | 2 h | * | NQ | NQ | NQ | NQ | * |
| 7 | 4 h | * | NQ | NQ | NQ | NQ | * |
| 7 | 8 h | * | NQ | NQ | NQ | NQ | * |
| 7 | 12 h | * | NQ | NQ | NQ | NQ | * |
| 7 | 24 h | * | NQ | NQ | NQ | NQ | * |

Key
NQ Non-quantifiable
Placebo sample, not analysed

CT327 Concentration (ngml⁻¹)
Sub No.

| Day | Time | R013 | R014 | R015 | R016 |
|---|---|---|---|---|---|
| 1 | predose | NQ | NQ | NQ | * |
| 1 | 0.5 h | NQ | NQ | NQ | * |
| 1 | 1 h | NQ | NQ | NQ | * |
| 1 | 2 h | NQ | NQ | NQ | * |
| 1 | 4 h | NQ | NQ | NQ | * |
| 1 | 12 h | NQ | NQ | NQ | * |
| 3 | predose | NQ | NQ | NQ | * |
| 3 | 0.5 h | NQ | NQ | NQ | * |
| 3 | 1 h | NQ | NQ | NQ | * |
| 3 | 2 h | NQ | NQ | NQ | * |
| 3 | 4 h | NQ | NQ | NQ | * |
| 3 | 12 h | NQ | NQ | NQ | * |
| 7 | predose | NQ | NQ | NQ | * |
| 7 | 0.5 h | NQ | NQ | NQ | * |
| 7 | 1 h | NQ | NQ | NQ | * |
| 7 | 2 h | NQ | NQ | NQ | * |
| 7 | 4 h | NQ | NQ | NQ | * |
| 7 | 8 h | NQ | NQ | NQ | * |
| 7 | 12 h | NQ | NQ | NQ | * |
| 7 | 24 h | NQ | NQ | NQ | * |

Key
NQ Non-quantifiable
Placebo sample. not analysed

TABLE 57
CT327 AMIDE SAMPLE ANALYSIS RESULTS

| | | CT327 Amide Concentration (ngml⁻¹) | | | | | |
|---|---|---|---|---|---|---|---|
| | | Sub | | | | | |
| | | | | | | | R006 |
| Day | Time | R001 | R002 | R003 | R004 | R005 | * |
| 1 | predose | NQ | NQ | NQ | NQ | NQ | * |
| 1 | 0.5 h | NQ | NQ | NQ | NQ | NQ | * |
| 1 | 1 h | NQ | NQ | NQ | NQ | NQ | * |
| 1 | 2 h | NQ | NQ | NQ | NQ | NQ | * |
| 1 | 4 h | NQ | NQ | NQ | NQ | NQ | * |
| 1 | 12 h | NQ | NQ | NQ | NQ | NQ | * |
| 3 | predose | NQ | NQ | NQ | NQ | NQ | * |
| 3 | 0.5 h | NQ | NQ | NQ | NQ | NQ | * |
| 3 | 1 h | NQ | NQ | NQ | NQ | NQ | * |
| 3 | 2 h | NQ | NQ | NQ | NQ | NQ | * |
| 3 | 4 h | NQ | NQ | NQ | NQ | NQ | * |
| 3 | 12 h | NQ | NQ | NQ | NQ | NQ | * |
| 7 | predose | NQ | NQ | NQ | NQ | NQ | * |
| 7 | 0.5 h | NQ | NQ | NQ | NQ | NQ | * |
| 7 | 1 h | NQ | NQ | NQ | NQ | NQ | * |
| 7 | 2 h | NQ | NQ | NQ | NQ | NQ | * |
| 7 | 4 h | NQ | NQ | NQ | NQ | NQ | * |
| 7 | 8 h | NQ | NQ | NQ | NQ | NQ | * |
| 7 | 12 h | NQ | NQ | NQ | NQ | NQ | * |
| 7 | 24 h | NQ | NQ | NQ | NQ | NQ | * |
| | | CT327 Amide Concentration (ngml⁻¹) | | | | | |
| | | Sub | | | | | |
| Day | Time | R007 | R008 | R009 | R010 | R011 | R012 |
| 1 | predose | * | NQ | NQ | NQ | NQ | * |
| 1 | 0.5 h | * | NQ | NQ | NQ | NQ | * |
| 1 | 1 h | * | NQ | NQ | NQ | NQ | * |
| 1 | 2 h | * | NQ | NQ | NQ | NQ | * |
| 1 | 4 h | * | NQ | NQ | NQ | NQ | * |
| 1 | 12 h | * | NQ | NQ | NQ | NQ | * |
| 3 | predose | * | NQ | NQ | NQ | NQ | * |
| 3 | 0.5 h | * | NQ | NQ | NQ | NQ | * |
| 3 | 1 h | * | NQ | NQ | NQ | NQ | * |
| 3 | 2 h | * | NQ | NQ | NQ | NQ | * |
| 3 | 4 h | * | NQ | NQ | NQ | NQ | * |
| 3 | 12 h | * | NQ | NQ | NQ | NQ | * |
| 7 | predose | * | NQ | NQ | NQ | NQ | * |
| 7 | 0.5 h | * | NQ | NQ | NQ | NQ | * |
| 7 | 1 h | * | NQ | NQ | NQ | NQ | * |
| 7 | 2 h | * | NQ | NQ | NQ | NQ | * |
| 7 | 4 h | * | NQ | NQ | NQ | NQ | * |
| 7 | 8 h | * | NQ | NQ | NQ | NQ | * |
| 7 | 12 h | * | NQ | NQ | NQ | NQ | * |
| 7 | 24 h | * | NQ | NQ | NQ | NQ | * |

Key
NO Non-quantifiable
Placebo sample, not analysed

CT327 Amide Concentration (ngml⁻¹)
Sub No.

| Day | Time | R013 | R014 | R015 | R016 |
|---|---|---|---|---|---|
| 1 | predose | NQ | NQ | NQ | * |
| 1 | 0.5 h | NQ | NQ | NQ | * |
| 1 | 1 h | NQ | NQ | NQ | * |
| 1 | 2 h | NQ | NQ | NQ | * |
| 1 | 4 h | NQ | NQ | NQ | * |
| 1 | 12 h | NQ | NQ | NQ | * |
| 3 | predose | NQ | NQ | NQ | * |
| 3 | 0.5 h | NQ | NQ | NQ | * |
| 3 | 1 h | NQ | NQ | NQ | * |
| 3 | 2 h | NQ | NQ | NQ | * |
| 3 | 4 h | NQ | NQ | NQ | * |
| 3 | 12 h | NQ | NQ | NQ | * |
| 7 | predose | NQ | NQ | NQ | * |
| 7 | 0.5 h | NQ | NQ | NQ | * |
| 7 | 1 h | NQ | NQ | NQ | * |
| 7 | 2 h | NQ | NQ | NQ | * |
| 7 | 4 h | NQ | NQ | NQ | * |
| 7 | 8 h | NQ | NQ | NQ | * |
| 7 | 12 h | NQ | NQ | NQ | * |
| 7 | 24 h | NQ | NQ | NQ | * |

Key
NO Non-quantifiable
Placebo sample. not analysed

### Incurred Sample Reanalysis

24 samples were reanalyzed to confirm reproducibility of the method. A minimum of two thirds of the reanalyzed samples had to show results within ±20% of their original value for the method to be classed as reproducible. Due to the nature of the samples, it was not possible to quantify these results. The results are presented in Tables 58 and 59.

TABLE 58
CT327 INCURRED SAMPLE REANALYSIS RESULTS

| Sample | Original | Repeat | % | |
|---|---|---|---|---|
| Code | Result | Result | Difference | Acceptable |
| 1A2 | NO | NO | — | — |
| 1A5 | NO | NO | — | — |
| 1A8 | NQ | NQ | — | — |
| 1Al2 | NQ | NQ | — | — |
| 2A5 | NO | NO | — | — |
| 2A11 | NQ | NQ | — | — |
| 2A16 | NQ | NQ | — | — |
| 2A19 | NO | NQ | — | — |
| 3A3 | NQ | NQ | — | — |
| 3A6 | NQ | NQ | — | — |
| 3A7 | NQ | NQ | — | — |
| 3A16 | NQ | NQ | — | — |
| 4A8 | NQ | NQ | — | — |
| 4A10 | NQ | NQ | — | — |
| 4A13 | NQ | NQ | — | — |
| 4A17 | NQ | NQ | — | — |
| 5A2 | NQ | NQ | — | — |
| 5A6 | NQ | NO | — | — |
| 5A8 | NQ | NQ | — | — |
| 5A14 | NQ | NQ | — | — |
| 8A5 | NQ | NQ | — | — |
| 8A8 | NQ | NO | — | — |
| 8A13 | NQ | NO | — | — |
| 8A16 | NQ | NQ | — | — |

Key
NQ Non-quantifiable

TABLE 59
CT327 AMIDE INCURRED SAMPLE REANALYSIS RESULTS

| Sample | Original | Repeat | % | |
|---|---|---|---|---|
| Code | Result | Result | Difference | Acceptable |
| 1A2 | NQ | NQ | — | — |
| 1A5 | NQ | NQ | — | — |
| 1A8 | NQ | NQ | — | — |
| 1AI2 | NQ | NQ | — | — |
| 2A5 | NQ | NQ | — | — |
| 2A11 | NQ | NQ | — | — |
| 2A16 | NQ | NQ | — | — |
| 2A19 | NQ | NQ | — | — |
| 3A3 | NQ | NQ | — | — |
| 3A6 | NQ | NQ | — | — |
| 3A7 | NQ | NQ | — | — |
| 3A16 | NQ | NQ | — | — |
| 4A8 | NQ | NQ | — | — |
| 4A10 | NQ | NQ | — | — |
| 4A13 | NQ | NQ | — | — |
| 4A17 | NQ | NQ | — | — |
| 5A2 | NQ | NQ | — | — |
| 5A6 | NQ | NQ | — | — |
| 5A8 | NQ | NQ | — | — |
| 5A14 | NQ | NQ | — | — |
| 8A5 | NQ | NQ | — | — |
| 8A8 | NQ | NQ | — | — |
| 8A13 | NQ | NQ | — | — |
| 8A16 | NQ | NQ | — | — |

Key
NQ Non-quantifiable

### Conclusions

240 samples from Phase I Study were successfully assayed for CT327 and CT327 amide using a validated HPLC-FLD method. 24 samples were reassayed and the original and reassayed results compared. The results from calibration curve standards, quality control samples and incurred sample reanalysis met the acceptance criteria for accuracy and precision, demonstrating acceptable performance of the method throughout the sample analysis period. Pharmacokinetic samples were analyzed, and surprisingly as there were no quantifiable concentrations of CT327 or CT327 amide in plasma for any of the patients in this study, indicating that there was either no systemic exposure to CT327 or CT327 amide or that systemic exposure was too low to be quantified using the current methodology.

### Pharmacokinetic Results—Determination of CT327 and CT327 Amide Levels in Human Urine Samples by HPLC-FLD

Summary

This project involved the determination of CT327 and CT327 amide in 84 human urine samples derived from a study involving the evaluation of the safety and tolerability of CT327 when administered twice daily, as a topical ointment, to lesions of psoriasis vulgaris in comparison to placebo.

The analytical procedure involved extraction of CT327 and CT327 amide from human urine by a liquid-liquid extraction. The human urine samples were analyzed using a validated HPLC method with fluorescence detection (FLD). Quantification was achieved using CT327 and CT327 amide to internal standard peak area ratios. Concentrations of the calibration curve standards, quality control samples and study samples were determined by the method of (1/x²) weighted least squares linear regression.

The lower limits of quantification for CT327 and CT327 amide were established at 10.00 ngml⁻¹ and 5.02 ngml⁻¹, respectively. The upper limits of quantification for CT327 and CT327 amide were established at 500.20 ngml⁻¹ and 251.11 ngml⁻¹, respectively. Calibration standards, quality control samples and the incurred sample reanalysis results met the acceptance criteria, demonstrating acceptable performance of the method during the analysis of the study samples. Pharmacokinetic samples were analyzed, but as there were no quantifiable concentrations of CT327 and CT327 amide in urine for any of the patients in this study, demonstrating that there was either no systemic exposure to CT327 or CT327 amide or that systemic exposure was too low to be quantified using the current methodology.

Materials

The analytical standards were CT327 (Serichim); CT327 amide (Serichim); and internal standard Midostaurin Hydrate (Sigma Aldrich).

For the biological matrix, blank (analyte-free) human urine was collected and employed.

A total of 84 human urine samples were analyzed. The samples were collected on day 1 pre-dose and 0-12 hours; day 3 pre-dose and 0-12 hours; and day 7 pre-dose, 0-12 and 12-24 hours after the 1 g dose of 0.5% w/w CT327 had been administered. A further 28 samples were received from subjects who had been dosed with placebo; these were not analyzed. All samples were stored at −80° C. (±10° C.).

Methods

The extraction procedure involved a liquid-liquid extraction. A portion of the extract was injected onto the HPLC-FLD system.

For calibration curve generation, duplicate calibrators at seven different concentrations, a human urine blank and a human urine blank plus internal standard were prepared for each batch. The calibration curves were produced by plotting the analyte to internal standard peak area ratios (y-axis) versus the concentration. The gradient and the intercept were determined by (1/x²) weighted least squares linear regression.

The concentration of CT327 and CT327 amide in each sample was calculated from the regression equation of the standard curve from each batch as follows:

Calculated concentration of analyte=((PAR-C)/M), where: PAR=analyte to internal standard peak area ratio; C=intercept; and M=gradient.

### Sample Analysis Results

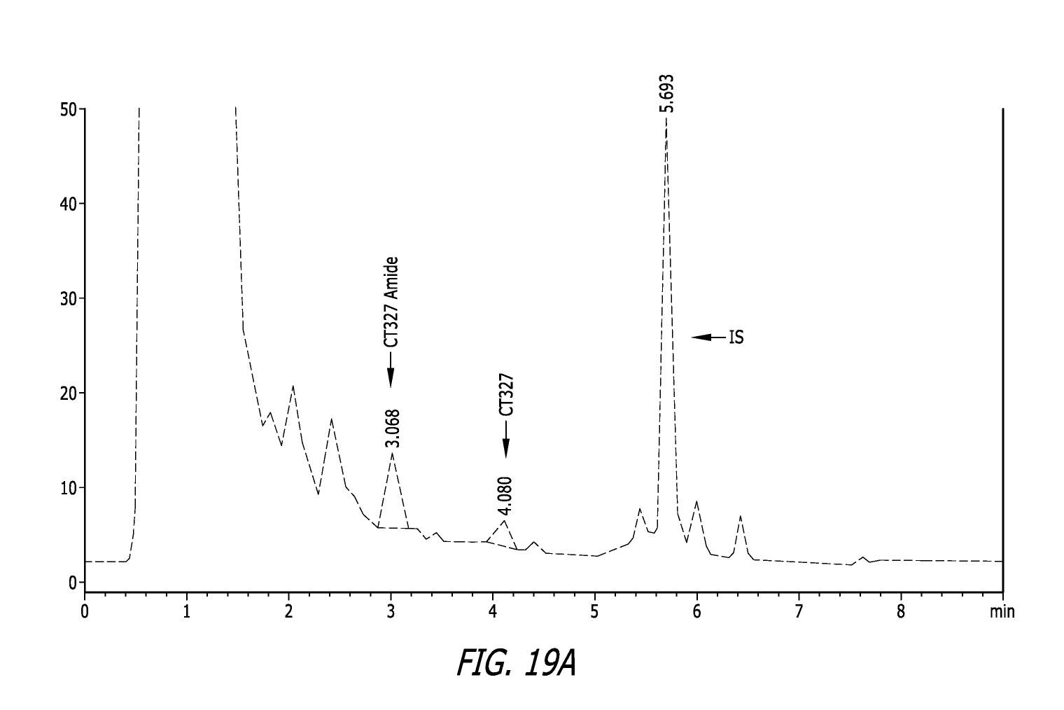

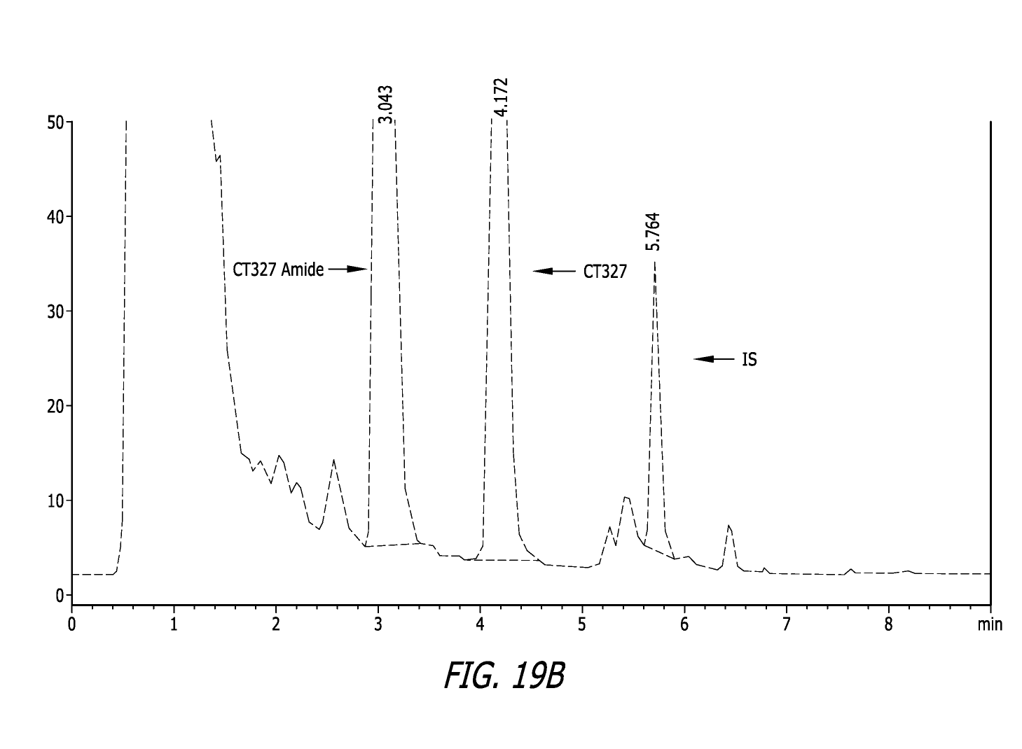

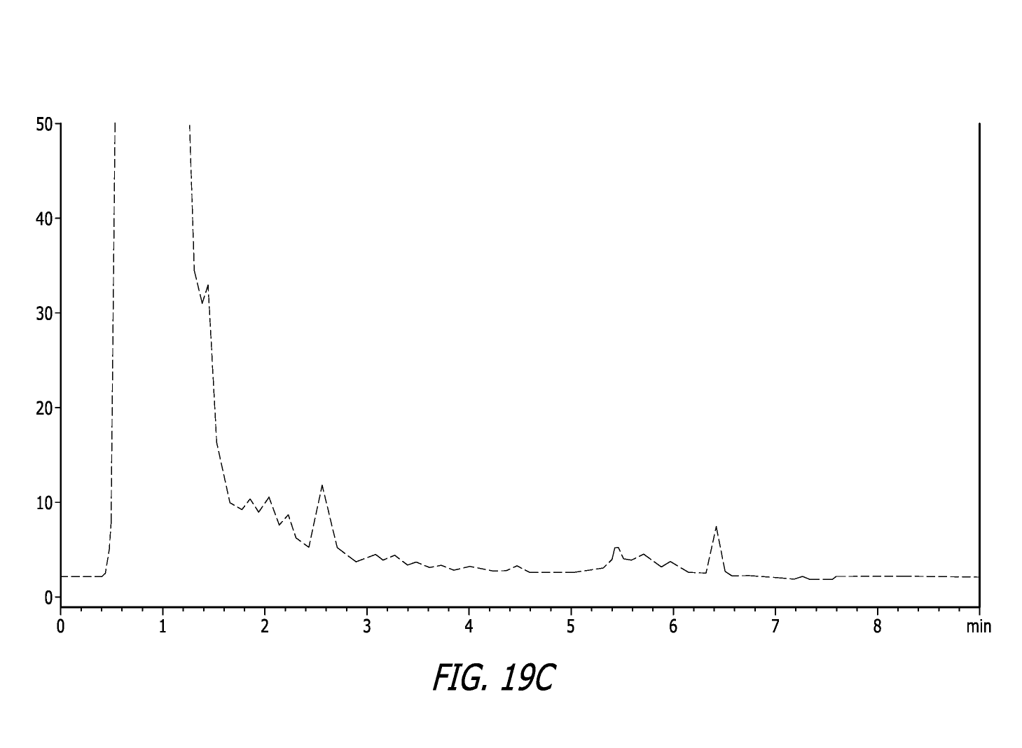

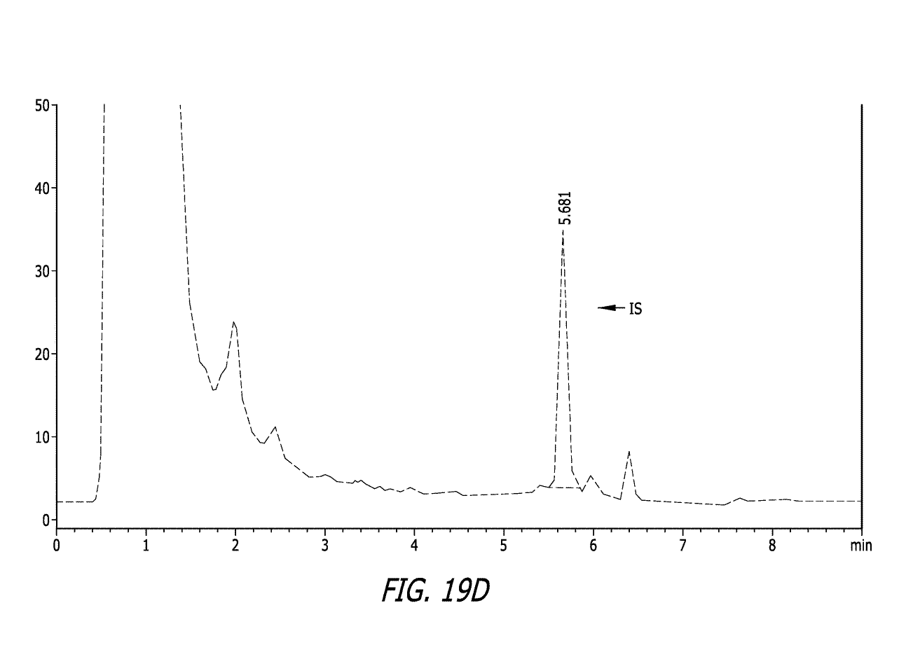

84 samples from 12 subjects (7 samples per subject) were analyzed by HPLC-FLD. The concentrations of CT327 and CT327 amide in the samples are presented in Tables 60 to 61, respectively. All samples for both analytes had concentrations below the LLOQ, and thus were reported as non-quantifiable (NQ). There were no failed batches during sample analysis. Typical chromatograms of the lower and upper limits of quantification, a blank sample, a patient urine sample are shown in FIGS. 19A to 19D, respectively.

TABLE 60
CT327 SAMPLE ANALYSIS RESULTS

CT327 Concentration (ngm1⁻¹)
Sub No.

| Day | Time | R001 | R002 | R003 | R004 |
|---|---|---|---|---|---|
| 1 | predose | NQ | NQ | NQ | NQ |
| 1 | 0-12 h | NQ | NQ | NQ | NQ |
| 3 | predose | NQ | NQ | NQ | NQ |
| 3 | 0-12 h | NQ | NQ | NQ | NQ |
| 7 | predose | NQ | NQ | NQ | NQ |
| 7 | 0-12 h | NQ | NQ | NQ | NQ |
| 7 | 12-24 h | NQ | NQ | NQ | NQ |

CT327 Concentration ngml⁻¹)
Sub No.

| Day | Time | R005 | R006 | R007 | R008 |
|---|---|---|---|---|---|
| 1 | predose | NQ | * | * | NQ |
| 1 | 0-12 h | NQ | * | * | NQ |
| 3 | predose | NQ | * | * | NQ |
| 3 | 0-12 h | NQ | * | * | NQ |
| 7 | predose | NQ | * | * | NQ |
| 7 | 0-12 h | NQ | * | * | NQ |
| 7 | 12-24 h | NQ | * | * | NQ |

CT327 Concentration (ngml⁻¹)
Sub No.

| Day | Time | R013 | R014 | R015 | R016 |
|---|---|---|---|---|---|
| 1 | predose | NQ | NQ | NQ | * |
| 1 | 0-12 h | NQ | NQ | NQ | * |
| 3 | predose | NQ | NQ | NQ | * |
| 3 | 0-12 h | NQ | NQ | NQ | * |
| 7 | predose | NQ | NQ | NQ | * |
| 7 | 0-12 h | NQ | NQ | NQ | * |
| 7 | 12-24 h | NQ | NQ | NQ | * |

CT327 Concentration (ngml⁻¹)
Sub No.

| Day | Time | R013 | R014 | R015 | R016 |
|---|---|---|---|---|---|
| 1 | predose | NQ | NQ | NQ | * |
| 1 | 0-12 h | NQ | NQ | NQ | * |
| 3 | predose | NQ | NQ | NQ | * |
| 3 | 0-12 h | NQ | NQ | NQ | * |
| 7 | predose | NQ | NQ | NQ | * |
| 7 | 0-12 h | NQ | NQ | NQ | * |
| 7 | 12-24 h | NQ | NQ | NQ | * |

Key
NQ Non-quantifiable
* Placebo sample not analysed

TABLE 61
CT327 AMIDE SAMPLE ANALYSIS RESULTS

| | | CT327 Amide Concentration (ngml⁻¹) | | | |
|---|---|---|---|---|---|
| | | Sub No. | | | |
| Day | Time | R001 | R002 | R003 | R004 |
| 1 | predose | NQ | NQ | NQ | NQ |
| 1 | 0-12 h | NQ | NQ | NQ | NQ |
| 3 | predose | NQ | NQ | NQ | NQ |
| 3 | 0-12 h | NQ | NQ | NQ | NQ |
| 7 | predose | NQ | NQ | NQ | NQ |
| 7 | 0-12 h | NQ | NQ | NQ | NQ |
| 7 | 12-24 h | NQ | NQ | NQ | NQ |
| | | CT327 Amide Concentration (ngml⁻¹) | | | |
| | | Sub No. | | | |
| Day | Time | R005 | R006 | R007 | R008 |
| 1 | predose | NQ | * | * | NQ |
| 1 | 0-12 h | NQ | * | * | NQ |
| 3 | predose | NQ | * | * | NQ |
| 3 | 0-12 h | NQ | * | * | NQ |
| 7 | predose | NQ | * | * | NQ |
| 7 | 0-12 h | NQ | * | * | NQ |
| 7 | 12-24 h | NQ | * | * | NQ |
| | | CT327 Amide Concentration (ngml⁻¹) | | | |
| | | Sub No. | | | |
| Day | Time | R009 | R010 | R011 | R012 |
| 1 | predose | NQ | NQ | NQ | * |
| 1 | 0-12 h | NQ | NQ | NQ | * |
| 3 | predose | NQ | NQ | NQ | * |
| 3 | 0-12 h | NQ | NQ | NQ | * |
| 7 | predose | NQ | NQ | NQ | * |
| 7 | 0-12 h | NQ | NQ | NQ | * |
| 7 | 12-24 h | NQ | NQ | NQ | * |
| | | CT327 Amide Concentration (ngml⁻¹) | | | |
| | | Sub No. | | | |
| Day | Time | R013 | R014 | R015 | R016 |
| 1 | predose | NQ | NQ | NQ | * |
| 1 | 0-12 h | NQ | NQ | NQ | * |
| 3 | predose | NQ | NQ | NQ | * |
| 3 | 0-12 h | NQ | NQ | NQ | * |
| 7 | predose | NQ | NQ | NQ | * |
| 7 | 0-12 h | NQ | NQ | NQ | * |
| 7 | 12-24 h | NQ | NQ | NQ | * |

Key
NQ Non-quantifiable
* Placebo sample not analysed

### Incurred Sample Reanalysis

8 samples were reanalyzed to confirm reproducibility of the method. A minimum of two thirds of the reanalyzed samples had to show results within ±20% of their original value for the method to be classed as reproducible. Due to the nature of the samples, it was not possible to quantify these results. The results are presented in Tables 62 and 63 for CT327 and CT327 amide, respectively.

TABLE 62
CT327 INCURRED SAMPLE REANALYSIS RESULTS

| | Original | Repeat | | |
|---|---|---|---|---|
| Sample Code | Result | Result | % Difference | Acceptable |
| 1A4 | NQ | NQ | — | — |
| 2A2 | NQ | NQ | — | — |
| 3A7 | NQ | NQ | — | — |
| 4A4 | NQ | NQ | — | — |
| 5A3 | NQ | NQ | — | — |
| 8A7 | NQ | NQ | — | — |
| 9A6 | NQ | NQ | — | — |
| 10A3 | NQ | NQ | — | — |

Key
NQ Non-quantifiable

TABLE 63
CT327 AMIDE INCURRED SAMPLE REANALYSIS RESULTS

| | Original | Repeat | | |
|---|---|---|---|---|
| Sample Code | Result | Result | % Difference | Acceptable |
| 1A4 | NQ | NQ | — | — |
| 2A2 | NQ | NQ | — | — |
| 3A7 | NQ | NQ | — | — |
| 4A4 | NQ | NQ | — | — |
| 5A3 | NQ | NQ | — | — |
| 8A7 | NQ | NQ | — | — |
| 9A6 | NQ | NQ | — | — |
| 10A3 | NQ | NQ | — | — |

Key
NQ Non-quantifiable

### Conclusions

84 samples from Sponsor Study No. CT327-1003 were successfully assayed for CT327 and CT327 amide, using a validated HPLC-FLD method. 8 samples were reassayed and the original and reassayed results compared. The results from calibration curve standards, quality control samples and incurred sample reanalysis met the acceptance criteria for accuracy and precision, demonstrating acceptable performance of the method throughout the sample analysis period. Pharmacokinetic samples were analyzed, and again, surprisingly, there were no quantifiable concentrations of CT327 or CT327 amide in urine for any of the patients in this study, indicating that there was either no systemic exposure to CT327 or CT327 amide or that systemic exposure was too low to be quantified using the current methodology.

### Efficacy Results

A summary of the PGA score is presented by study treatment in Table 64. No patients showed complete resolution of their plaque(s) during the short dosing period of the study. Generally, PGA scores for induration, erythema, scaling and physician's static global score improved for all patients from baseline to Day 7, regardless of which treatment group they were in.

TABLE 64
SUMMARY OF PGA SCORES BY TREATMENT GROUP

| | | | CT327 | Placebo | Overall |
|---|---|---|---|---|---|
| Parameter | Visit | Assesment | (N = 12) | (N = 4) | (N = 16) |
| Plaque 1 | | | | | |

| Induration | SCREENING | 1: MINIMAL PLAQUE ELEVATION, =0.25 MM | 4 | (33.3%) | 2 | (50.0%) | 6 | (37.5%) |
|---|---|---|---|---|---|---|---|---|
| | | 2: MILD PLAQUE ELEVATION, =0.5 MM | 6 | (50.0%) | 1 | (25.0%) | 7 | (43.8%) |
| | | 3: MODERATE PLAQUE ELEVATION, =0.75 MM | 1 | (8.3%) | 1 | (25.0%) | 2 | (12.5%) |
| | | 4: MARKED PLAQUE ELEVATION, =1 MM | 1 | (8.3%) | 0 | | 1 | (6.3%) |
| | DAY 1 | 1: MINIMAL PLAQUE ELEVATION, =0.25 MM | 4 | (33.3%) | 1 | (25.0%) | 5 | (31.3%) |
| | | 2: MILD PLAQUE ELEVATION, =0.5 MM | 5 | (41.7%) | 1 | (25.0%) | 6 | (37.5%) |
| | | 3: MODERATE PLAQUE ELEVATION, =0.75 MM | 3 | (25.0%) | 2 | (50.0%) | 5 | (31.3%) |
| | DAY 7 | 1: MINIMAL PLAQUE ELEVATION, =0.25 MM | 8 | (66.7%) | 2 | (50.0%) | 10 | (62.5%) |
| | | 2: MILD PLAQUE ELEVATION, =0.5 MM | 4 | (33.3%) | 1 | (25.0%) | 5 | (31.3%) |
| | | 3: MODERATE PLAQUE ELEVATION, =0.75 MM | 0 | | 1 | (25.0%) | 1 | (6.3%) |
| | FOLLOW-UP | 1: MINIMAL PLAQUE ELEVATION, =0.25 MM | 4 | (33.3%) | 3 | (75.0%) | 7 | (43.8%) |
| | | 2: MILD PLAQUE ELEVATION, =0.5 MM | 8 | (66.7%) | 1 | (25.0%) | 9 | (56.3%) |
| Erythma | SCREENING | 1: FAINT ERYTHEMA | 1 | (8.3%) | 0 | | 1 | (6.3%) |
| | | 2: LIGHT RED COLOURATION | 5 | (41.7%) | 4 | (100%) | 9 | (56.3%) |
| | | 3: MODERATE RED COLOURATION | 5 | (41.7%) | 0 | | 5 | (31.3%) |
| | | 4: BRIGHT RED COLOURATION | 1 | (8.3%) | 0 | | 1 | (6.3%) |
| | DAY 1 | 1: FAINT ERYTHEMA | 2 | (16.7%) | 2 | (50.0%) | 4 | (25.0%) |
| | | 2: LIGHT RED COLOURATION | 8 | (66.7%) | 2 | (50.0%) | 10 | (62.5%) |
| | | 3: MODERATE RED COLOURATION | 1 | (8.3%) | 0 | | 1 | (6.3%) |
| | | 4: BRIGHT RED COLOURATION | 1 | (8.3%) | 0 | | 1 | (6.3%) |
| | DAY 7 | 1: FAINT ERYTHEMA | 2 | (16.7%) | 2 | (50.0%) | 4 | (25.0%) |
| | | 2: LIGHT RED COLOURATION | 8 | (66.7%) | 2 | (50.0%) | 10 | (62.5%) |
| | | 3: MODERATE RED COLOURATION | 1 | (8.3%) | 0 | | 1 | (6.3%) |
| | | 4: BRIGHT RED COLOURATION | 1 | (8.3%) | 0 | | 1 | (6.3%) |
| | FOLLOW-UP | 1: FAINT ERYTHEMA | 4 | (33.3%) | 2 | (50.0%) | 6 | (37.5%) |
| | | 2: LIGHT RED COLOURATION | 7 | (58.3%) | 2 | (50.0%) | 9 | (56.3%) |
| | | 4: BRIGHT RED COLOURATION | 1 | (8.3%) | 0 | | 1 | (6.3%) |
| Scaling | SCREENING | 1: MINIMAL, OCCASIONAL FINE SCALE OVER | 2 | (16.7%) | 1 | (25.0%) | 3 | (18.8%) |
| | | LESS THAN 5% OF THE LESION | | | | | | |
| | | 2: MILD, FINE SCALE DOMINATES | 4 | (33.3%) | 2 | (50.0%) | 6 | (37.5%) |
| | | 3: MODERATE, COARSE SCALE | 5 | (41.7%) | 1 | (25.0%) | 6 | (37.5%) |
| | | PREDOMINATES | | | | | | |
| | | 4: MARKED, THICK, NON-TENACIOUS SCALE | 1 | (8.3%) | 0 | | 1 | (6.3%) |
| | | DOMINATES | | | | | | |
| | DAY 1 | 2: MILD, FINE SCALE DOMINATES | 7 | (58.3%) | 4 | (100%) | 11 | (68.8%) |
| | | 3: MODERATE, COARSE SCALE | 4 | (33.3%) | 0 | | 4 | (25.0%) |
| | | PREDOMINATES | | | | | | |
| | | 4: MARKED, THICK, NON-TENACIOUS SCALE | 1 | (8.3%) | 0 | | 1 | (6.3%) |
| | | DOMINATES | | | | | | |
| | DAY 7 | 1: MINIMAL, OCCASIONAL FINE SCALE OVER | 7 | (58.3%) | 2 | (50.0%) | 9 | (56.3%) |
| | | LESS THAN | | | | | | |
| | | 5% OF THE LESION | | | | | | |
| | | 2: MILD, FINE SCALE DOMINATES | 4 | (33.3%) | 0 | | 4 | (25.0%) |
| | | 0: NO EVIDENCE OF SCALING | 1 | (8.3%) | 2 | (50.0%) | 3 | (18.8%) |
| | FOLLOW-UP | 1: MINIMAL, OCCASIONAL FINE SCALE OVER | 2 | (16.7%) | 2 | (50.0%) | 4 | (25.0%) |
| | | LESS THAN | | | | | | |
| | | 5% OF THE LESION | | | | | | |
| | | 2: MILD, FINE SCALE DOMINATES | 7 | (58.3%) | 1 | (25.0%) | 8 | (50.0%) |
| | | 3: MODERATE, COARSE SCALE | 3 | (25.0%) | 0 | | 3 | (18.8%) |
| | | PREDOMINATES | | | | | | |
| | | 0: NO EVIDENCE OF SCALING | 0 | | 1 | (25.0%) | 1 | (6.3%) |
| Physician Static | SCREENING | 1: MINIMAL - MAJORITY OF LESIONS HAVE | 2 | (16.7%) | 1 | (25.0%) | 3 | (18.8%) |
| Global Score | | INDIVIDUAL SCORES FOR I + E + S/3 THAT | | | | | | |
| | | AVERAGED 1 | | | | | | |
| | | 2: MILD - MAJORITY OF LESIONS HAVE | 5 | (41.7%) | 2 | (50.0%) | 7 | (43.8%) |
| | | INDIVIDUAL SCORES FOR I + E + S/3 THAT | | | | | | |
| | | AVERAGES 2 | | | | | | |
| | | 3: MODERATE - MAJORITY OF LESIONS HAVE | 4 | (33.3%) | 1 | (25.0%) | 5 | (31.3%) |
| | | INDIVIDUAL SCORES FOR I + E + S/3 THAT | | | | | | |
| | | AVERAGES 3 | | | | | | |
| | | 4: MARKED - MAJORITY OF LESIONS HAVE | 1 | (8.3%) | 0 | | 1 | (6.3%) |
| | | INDIVIDUAL SCORES FOR I + E + S/3 THAT | | | | | | |
| | | AVERAGES 4 | | | | | | |
| | DAY 1 | 1: MINIMAL - MAJORITY OF LESIONS HAVE | 1 | (8.3%) | 1 | (25.0%) | 2 | (12.5%) |
| | | INDIVIDUAL SCORES FOR I + E + S/3 THAT | | | | | | |
| | | AVERAGED 1 | | | | | | |
| Physician Static | DAY 1 | 2: MILD - MAJORITY OF LESIONS HAVE | 7 | (58.3%) | 3 | (75.0%) | 10 | (62.5%) |
| Global Score | | INDIVIDUAL SCORES FOR I + E + S/3 THAT | | | | | | |
| | | AVERAGES 2 | | | | | | |
| | | 3: MODERATE - MAJORITY OF LESIONS | 4 | (33.3%) | 0 | | 4 | (25.0%) |
| | | HAVE INDIVIDUAL SCORES FOR I + E + S/3 | | | | | | |
| | | THAT AVERAGES 3 | | | | | | |
| | DAY 7 | 1: MINIMAL - MAJORITY OF LESIONS HAVE | 5 | (41.7%) | 3 | (75.0%) | 8 | (50.0%) |
| | | INDIVIDUAL SCORES FOR I + E + S/3 THAT | | | | | | |
| | | AVERAGED 1 | | | | | | |
| | | 2: MILD - MAJORITY OF LESIONS HAVE | 6 | (50.0%) | 1 | (25.0%) | 7 | (43.8%) |
| | | INDIVIDUAL SCORES FOR I + E + S/3 THAT | | | | | | |
| | | AVERAGES 2 | | | | | | |
| | | 3: MODERATE - MAJORITY OF LESIONS | 1 | (8.3%) | 0 | | 1 | (6.3%) |
| | | HAVE INDIVIDUAL SCORES FOR I + E + S/3 | | | | | | |
| | | THAT AVERAGES 3 | | | | | | |
| | FOLLOW-UP | 1: MINIMAL - MAJORITY OF LESIONS HAVE | 2 | (16.7%) | 3 | (75.0%) | 5 | (31.3%) |
| | | INDIVIDUAL SCORES FOR I + E + S/3 THAT | | | | | | |
| | | AVERAGED 1 | | | | | | |
| | | 2: MILD - MAJORITY OF LESIONS HAVE | 9 | (75.0%) | 1 | (25.0%) | 10 | (62.5%) |
| | | INDIVIDUAL SCORES FOR I + E + S/3 THAT | | | | | | |
| | | AVERAGES 2 | | | | | | |

| | | | | | |
|---|---|---|---|---|---|
| 3: MODERATE - MAJORITY OF LESIONS | 1 | (8.3%) | 0 | 1 | (6.3%) |
| HAVE INDIVIDUAL SCORES FOR I + E + S/3 | | | | | |
| THAT AVERAGES 3 | | | | | |

| | | | CT327 | Placebo | Overall |
|---|---|---|---|---|---|
| Parameter | Visit | Assesment | (N= 12) 1 | (N = 4) | (N = 16) |
| Plaque 2 | | | | | |

| | | | | | | | |
|---|---|---|---|---|---|---|---|
| Induration | SCREENING | 1: MINIMAL PLAQUE ELEVATION, =0.25 MM | 1 | (8.3%) | 0 | 1 | (6.3%) |
| | | 2: MILD PLAQUE ELEVATION, =0.5 MM | 4 | (33.3%) | 0 | 4 | (25.0%) |
| | | 3: MODERATE PLAQUE ELEVATION, =0.75 MM | 1 | (8.3%) | 0 | 1 | (6.3%) |
| | | 4: MARKED PLAQUE ELEVATION, =1 MM | 1 | (8.3%) | 0 | 1 | (6.3%) |
| | DAY 1 | 1: MINIMAL PLAQUE ELEVATION, =0.25 MM | 1 | (8.3%) | 0 | 1 | (6.3%) |
| | | 2: MILD PLAQUE ELEVATION, =0.5 MM | 2 | (16.7%) | 0 | 2 | (12.5%) |
| | | 3: MODERATE PLAQUE ELEVATION, =0.75 MM | 1 | (8.3%) | 0 | 1 | (6.3%) |
| | DAY 7 | 1: MINIMAL PLAQUE ELEVATION, =0.25 MM | 3 | (25.0%) | 0 | 3 | (18.8%) |
| | | 2: MILD PLAQUE ELEVATION, =0.5 MM | 1 | (8.3%) | 0 | 1 | (6.3%) |
| | FOLLOW-UP | 1: MINIMAL PLAQUE ELEVATION, =0.25 MM | 1 | (8.3%) | 0 | 1 | (6.3%) |
| | | 2: MILD PLAQUE ELEVATION, =0.5 MM | 3 | (25.0%) | 0 | 3 | (18.8%) |
| Erythma | SCREENING | 2: LIGHT RED COLOURATION | 4 | (33.3%) | 0 | 4 | (25.0%) |
| | | 3: MODERATE RED COLOURATION | 3 | (25.0%) | 0 | 3 | (18.8%) |
| | DAY 1 | 2: LIGHT RED COLOURATION | 4 | (33.3%) | 0 | 4 | (25.0%) |
| | DAY 7 | 2: LIGHT RED COLOURATION | 3 | (25.0%) | 0 | 3 | (18.8%) |
| | | 3: MODERATE RED COLOURATION | 1 | (8.3%) | 0 | 1 | (6.3%) |
| | FOLLOW-UP | 2: LIGHT RED COLOURATION | 4 | (33.3%) | 0 | 4 | (25.0%) |
| Scaling | SCREENING | 2: MILD, FINE SCALE DOMINATES | 2 | (16.7%) | 0 | 2 | (12.5%) |
| | | 3: MODERATE, COARSE SCALE PREDOMINATES | 4 | (33.3%) | 0 | 4 | (25.0%) |
| | | 4: MARKED, THICK, NON-TENACIOUS SCALE DOMINATES | 1 | (8.3%) | 0 | 1 | (6.3%) |
| | DAY 1 | 2: MILD, FINE SCALE DOMINATES | 2 | (16.7%) | 0 | 2 | (12.5%) |
| | | 3: MODERATE, COARSE SCALE PREDOMINATES | 1 | (8.3%) | 0 | 1 | (6.3%) |
| | | 4: MARKED, THICK, NON-TENACIOUS SCALE DOMINATES | 1 | (8.3%) | 0 | 1 | (6.3%) |
| | DAY 7 | 2: MILD, FINE SCALE DOMINATES | 2 | (16.7%) | 0 | 2 | (12.5%) |
| | | 1: MINIMAL, OCCASIONAL FINE SCALE OVER LESS THAN | 2 | (16.7%) | 0 | 2 | (12.5%) |
| | | 5% OF THE LESION | | | | | |
| | FOLLOW-UP | 2: MILD, FINE SCALE DOMINATES | 3 | (25.0%) | 0 | 3 | (18.8%) |

| | | | CT327 | | Placebo | Overall | |
|---|---|---|---|---|---|---|---|
| Parameter | Visit | Assesment | (N = 12) | | (N = 4) | (N = 16) | |
| Scaling | FOLLOW-UP | 1: MINIMAL, OCCASIONAL FINE SCALE OVER LESS | 1 | (8.3%) | 0 | 1 | (6.3%) |
| | | THAN 5% OF THE LESION | | | | | |
| Physician | SCREENING | 2: MILD - MAJORITY OF LESIONS HAVE INDIVIDUAL | 4 | (33.3%) | 0 | 4 | (25.0%) |
| Static Global | | SCORES FOR I + E + S/3 THAT AVERAGES 2 | | | | | |
| Score | | 3: MODERATE - MAJORITY OF LESIONS HAVE | 2 | (16.7%) | 0 | 2 | (12.5%) |
| | | INDIVIDUAL SCORES FOR I + E + S/3 THAT | | | | | |
| | | AVERAGES 3 | | | | | |
| | | 4: MARKED - MAJORITY OF LESIONS HAVE | 1 | (8.3%) | 0 | 1 | (6.3%) |
| | | INDIVIDUAL SCORES FOR I + E + S/3 THAT | | | | | |
| | | AVERAGES 4 | | | | | |
| | DAY 1 | 2: MILD - MAJORITY OF LESIONS HAVE INDIVIDUAL | 3 | (25.0%) | 0 | 3 | (18.8%) |
| | | SCORES FOR I + E + S/3 THAT AVERAGES 2 | | | | | |
| | | 3: MODERATE - MAJORITY OF LESIONS HAVE | 1 | (8.3%) | 0 | 1 | (6.3%) |
| | | INDIVIDUAL SCORES FOR I + E + S/3 THAT | | | | | |
| | | AVERAGES 3 | | | | | |
| | DAY 7 | 2: MILD - MAJORITY OF LESIONS HAVE INDIVIDUAL | 3 | (25.0%) | 0 | 3 | (18.8%) |
| | | SCORES FOR I + E + S/3 THAT AVERAGES 2 | | | | | |
| | | 1: MINIMAL - MAJORITY OF LESIONS HAVE | 1 | (8.3%) | 0 | 1 | (6.3%) |
| | | INDIVIDUAL SCORES FOR I + E + S/3 THAT | | | | | |
| | | AVERAGED 1 | | | | | |
| | FOLLOW-UP | 2: MILD - MAJORITY OF LESIONS HAVE INDIVIDUAL | 4 | (33.3%) | 0 | 4 | (25.0%) |
| | | SCORES FOR I + E + S/3 THAT AVERAGES 2 | | | | | |
| Plaque 3 | | | | | | | |
| Induration | SCREENING | 2: MILD PLAQUE ELEVATION, =0.5 MM | 1 | (8.3%) | 0 | 1 | (6.3%) |
| | | 3: MODERATE PLAQUE ELEVATION, =0.75 MM | 1 | (8.3%) | 0 | 1 | (6.3%) |
| | | 4: MARKED PLAQUE ELEVATION, =1 MM | 1 | (8.3%) | 0 | 1 | (6.3%) |
| | DAY 1 | 2: MILD PLAQUE ELEVATION, =0.5 MM | 1 | (8.3%) | 0 | 1 | (6.3%) |
| | | 3: MODERATE PLAQUE ELEVATION, =0.75 MM | 1 | (8.3%) | 0 | 1 | (6.3%) |
| | DAY 7 | 1: MINIMAL PLAQUE ELEVATION, =0.25 MM | 2 | (16.7%) | 0 | 2 | (12.5%) |
| | FOLLOW-UP | 2: MILD PLAQUE ELEVATION, =0.5 MM | 1 | (8.3%) | 0 | 1 | (6.3%) |
| | | 1: MINIMAL PLAQUE ELEVATION, =0.25 MM | 1 | (8.3%) | 0 | 1 | (6.3%) |
| Erythma | SCREENING | 2: LIGHT RED COLOURATION | 1 | (8.3%) | 0 | 1 | (6.3%) |
| | | 3: MODERATE RED COLOURATION | 2 | (16.7%) | 0 | 2 | (12.5%) |
| | DAY 1 | 2: LIGHT RED COLOURATION | 2 | (16.7%) | 0 | 2 | (12.5%) |
| | DAY 7 | 2: LIGHT RED COLOURATION | 1 | (8.3%) | 0 | 1 | (6.3%) |
| | | 3: MODERATE RED COLOURATION | 1 | (8.3%) | 0 | 1 | (6.3%) |
| | FOLLOW-UP | 2: LIGHT RED COLOURATION | 2 | (16.7%) | 0 | 2 | (12.5%) |
| Scaling | SCREENING | 3: MODERATE, COARSE SCALE PREDOMINATES | 2 | (16.7%) | 0 | 2 | (12.5%) |
| | | 4: MARKED, THICK, NON-TENACIOUS SCALE | 1 | (8.3%) | 0 | 1 | (6.3%) |
| | | DOMINATES | | | | | |
| | DAY 1 | 4: MARKED, THICK, NON-TENACIOUS SCALE | 1 | (8.3%) | 0 | 1 | (6.3%) |
| | | DOMINATES | | | | | |
| | | 2: MILD, FINE SCALE DOMINATES | 1 | (8.3%) | 0 | 1 | (6.3%) |
| | DAY 7 | 2: MILD, FINE SCALE DOMINATES | 2 | (16.7%) | 0 | 2 | (12.5%) |
| | FOLLOW-UP | 2: MILD, FINE SCALE DOMINATES | 2 | (16.7%) | 0 | 2 | (12.5%) |
| Physician Static | SCREENING | 2: MILD - MAJORITY OF LESIONS HAVE | 1 | (8.3%) | 0 | 1 | (6.3%) |
| Global Score | | INDIVIDUAL SCORES FOR I + E + S/3 THAT | | | | | |
| | | AVERAGES 2 | | | | | |
| | | 3: MODERATE - MAJORITY OF LESIONS HAVE | 1 | (8.3%) | 0 | 1 | (6.3%) |
| | | INDIVIDUAL SCORES FOR I + E + S/3 THAT | | | | | |
| | | AVERAGES 3 | | | | | |
| Physician Static | SCREENING | 4: MARKED - MAJORITY OF LESIONS HAVE | 1 | (8.3%) | 0 | 1 | (6.3%) |
| Global Score | | INDIVIDUAL SCORES FOR I + E + S/3 THAT | | | | | |
| | | AVERAGES 4 | | | | | |
| | DAY 1 | 2: MILD - MAJORITY OF LESIONS HAVE | 1 | (8.3%) | 0 | 1 | (6.3%) |
| | | INDIVIDUAL SCORES FOR I + E + S/3 THAT | | | | | |
| | | AVERAGES 2 | | | | | |
| | | 3: MODERATE - MAJORITY OF LESIONS HAVE | 1 | (8.3%) | 0 | 1 | (6.3%) |
| | | INDIVIDUAL SCORES FOR I + E + S/3 THAT | | | | | |
| | | AVERAGES 3 | | | | | |
| | DAY 7 | 2: MILD - MAJORITY OF LESIONS HAVE | 2 | (16.7%) | 0 | 2 | (12.5%) |
| | | INDIVIDUAL SCORES FOR I + E + S/3 THAT | | | | | |
| | | AVERAGES 2 | | | | | |
| | FOLLOW-UP | 2: MILD - MAJORITY OF LESIONS HAVE | 2 | (16.7%) | 0 | 2 | (12.5%) |
| | | INDIVIDUAL SCORES FOR I + E + S/3 THAT | | | | | |
| | | AVERAGES 2 | | | | | |

### Safety Evaluation

All patients randomized onto the study received study treatment and completed the dosing schedule as per protocol.

Summary of Treatment-Emergent Adverse Events

An overall summary of TEAEs by treatment is summarized in Table 65.

Six patients (50%) in the CT327 treatment group reported 10 TEAEs during the study and one patient (25%) in the placebo group reported 3 TEAEs. The most commonly reported TEAE was headache, 5 occurrences reported by 3 patients (25%), all in the CT327 treatment group. Psoriasis was reported as a TEAE by 2 patients (16.7%) in the CT327 treatment group. All other TEAEs were reported by one patient each (otitis media acute, mouth ulceration, sunburn, back pain, muscle twitching and rhinalgia).

The maximum intensity of TEAEs was classified as mild for 11 events (8 TEAEs in the CT327 treatment group and 3 TEAEs in the placebo group) and moderate for 2 events (both in the CT327 treatment group). No severe TEAEs were reported.

There were 8 pre-treatment AEs reported, 5 patients (31.3%) in the CT327 treatment group reported 8 AEs and 3 patients (18.8%) in the placebo group reported 3 AEs.

No AEs were noted at the treatment application sites.

TABLE 65
OVERVIEW OF TEAEs

Treatment Group

| | | |
|---|---|---|
| TEAE Category | CT327 | Placebo |

| | | | |
|---|---|---|---|
| TEAE | 6 | (50.0) [10] | 1 (25.0) [3] |
| Mild TEAE | 5 | (41.7) [8] | 1 (25.0) [3] |
| Moderate TEAE | 2 | (16.7) [2] | 0 |

| | | |
|---|---|---|
| Severe TEAE | 0 | 0 |

| | | | |
|---|---|---|---|
| Related TEAE | 1 | (8.3) [1] | 0 |

| | | |
|---|---|---|
| TEAE leading to discontinuation | 0 | 0 |
| SAE | 0 | 0 |

Note:
x (xx.x%): x = number of patients experiencing the event, percentages (xx.x%) are calculated as 100 × (x/number of patients treated).
n = number of patients; % = percentage of patients with the event; [m] = number of events.

### Discussion and Overall Conclusions

The primary objective of this study was to evaluate the safety and tolerability of CT327 when administered bid as a topical ointment to lesions of psoriasis vulgaris in comparison to placebo. No deaths, SAEs or withdrawals due to TEAEs were reported in either treatment group. A higher percentage of patients (50%) reported TEAEs in the CT327 treatment group compared to 25% of patients in the placebo group. Only 2 moderate TEAEs were reported, both in the CT327 treatment group. No severe TEAEs were reported in either treatment group. The most commonly reported TEAE was headache, 5 occurrences reported by 3 patients (25%), all in the CT327 treatment group. Psoriasis was reported as a TEAE by 2 patients (16.7%) in the CT327 treatment group. All other TEAEs were reported by one patient each.

There were no quantifiable concentrations of CT327 or its degradation product CT327 amide. This indicates that there was either no systemic exposure to CT327 or CT327 amide or that systemic exposure was too low to be quantified using the current methodology.

Generally, PGA scores for induration, erythema, scaling and physician's static global score improved for all patients from baseline to Day 7, regardless of which treatment group they were in.

In conclusion, study treatment was well tolerated by both treatment groups and there was little difference in the safety and tolerability between CT327 administration or placebo when administered bid as a topical ointment to lesions of psoriasis vulgaris. No AEs were noted at the treatment application sites. Further, there were no clinically significant findings in clinical laboratory test results, vital signs, ECGs or physical examinations during the study, in either treatment group. Additionally, there were no quantifiable concentrations of CT327 or its degradation product CT327 amide. And finally, PGA score results showed no differences between treatment groups.

### Example 9: Pharmacokinetics and Safety Associated with Maximal Use of 0.5% W/W SNA-120 (Pegcantratinib) Ointment Administered Twice Daily in Subjects with Pruritus Associated with Psoriasis Vulgaris

This study, using an assay that is five times more sensitive than previously available, demonstrated minimal to no systemic exposure after twice daily topical application of SNA-120 (see Table 1) under maximal use conditions, with no accumulation of SNA-120 in the plasma. There were no associated clinically relevant changes in the overall safety parameters evaluated, including no systemic safety signals (i.e. laboratory assessments, electrocardiograms (ECG), QTc duration, and adverse events (AEs)). A total of 9 subjects reported 12 adverse events (AEs), only one of which was deemed related to study drug and which was categorized as mild pruritus. There were also no dermal tolerability events reported, no subject discontinuations due to an AE, and no severe AEs or serious AEs (SAEs). The majority of subjects had no detectable levels of SNA-120 in the plasma (66.7% and 67.9% of subjects, respectively, on Day 1 and 29). Low (less than 2.5 ng/mL) detectable levels of SNA-120 in the plasma were observed in 30.0% and 28.6% of subjects, respectively, on Day 1 and 29. No accumulation of SNA-120 in the plasma was seen following repeated twice-daily topical application. SNA-120 was well tolerated with no subjects discontinuing the study due to Adverse Events (AE). Improvements in pruritus and psoriasis were observed in exploratory efficacy analyses. For example, in a post hoc analysis, approximately 57 percent of subjects had at least a 4-grade improvement in the Itch Numeric Rating Scale (I-NRS).

Chronic itch affects roughly 80 percent, or 6.3 million (Global Data (2016). PharmaPoint: Psoriasis—Global Drug Forecast), of the 7.9 million patients suffering from psoriasis in the United States (Mentor et al. Guidelines of care for the management of psoriasis and psoriatic arthritis. J Am Acad Dermatol. 2008; 58(5):826-850). It is an important—and often overlooked—symptom in patients with psoriasis. One study found that 50% of patients said they had difficulty sleeping due to itch and 75% reported having to scratch until they bleed (Prignano F, Ricceri F, Pescitelli L, Lotti T. Itch in psoriasis: epidemiology, clinical aspects and treatment options. Clin Cosmet Investig Dermatol). The itch that affects the majority of psoriasis patients can be as debilitating and harmful to their quality of life as the plaques themselves, yet there are no FDA-approved, targeted, topical medications available that are addressing this bothersome itch that could extend beyond the plaque.

This is a multicenter, open-label, Phase 1b study designed to evaluate the PK with maximal use of 0.5% w/w SNA-120 ointment (see Table 1), as measured by circulating plasma levels of SNA-120 in men and women aged ≥18 years who have at least moderate (e.g., moderate to severe) pruritus associated with at least moderate (e.g., moderate to severe) psoriasis vulgaris (PV) covering at least 20% body surface area (BSA). SNA-120 was evaluated as a topical, non-steroidal ointment to treat itch (pruritus) associated with psoriasis. SNA-120 was designed to achieve high local drug concentration in the target tissue while minimizing systemic exposure for patients. SNA-120 directly targets the peripheral itch pathway in the psoriatic plaque.

One objective of the study is to evaluate the pharmacokinetics (PK) with maximal use of 0.5% weight-to-weight ratio (w/w) SNA-120 ointment as measured by circulating plasma levels of SNA-120 in subjects with chronic pruritus associated with psoriasis vulgaris (plaque psoriasis). Another objective is to evaluate the effect of maximum therapeutic dose of SNA-120 treatment on Fridericia's corrected QT interval (QTcF).

Approximately 30 eligible subjects will receive 0.5% w/w SNA-120 ointment (2 mg/cm²) proportional to approximately 35 mg active drug at each application for a subject with at least 20% BSA affected, administered twice daily over 4 weeks to the indicated areas in the Treatment Area. The “Treatment Area” is defined as the BSA (including the face, scalp, intertriginous, and genital areas) exclusive of the mucosa, palms, and soles. The “Affected Areas” to be treated within the Treatment Area shall include: 1) all psoriasis (e.g., plaques) present in the Treatment Area at Baseline; 2) any new or recurrent areas of psoriasis that may develop during the study; and 3) the original plaque area, even in the event that the psoriasis (erythema, induration, scale) resolves and the lesions are no longer clearly visible (aside from hyper/hypo pigmentation). Subjects should continue to apply study drug to current active plaques, resolved/resolving plaques (residual hyper/hypopigmentation), or newly appearing plaques for the full 4 weeks of treatment, always covering a minimum of 20% BSA.

On Study Days 1 and 29 subjects will be asked to assume the supine position and remain quiet but awake for at least 20 minutes around each nominal electrocardiogram (ECG) extraction point (15 minutes before the time point until 5 minutes after each time point). Once the ECG extraction is started (time: 0 h), the subject should remain at rest in the supine position for the next 5 minutes, with the exception of PK draws. The extraction time points from the continuous ECG monitoring of the QT interval corrected for heart rate (QTc) analysis are as follows: predose (within 1 hour of the start of dosing), and 0.5, 0.75, 1, 2, 4, 8, and 12 hours after the end of the SNA-120 application.

The average of triplicate ECG measurements from each time point will serve as each subject's QT value. Electrocardiograms will be measured using semi-automated methods for the ECG intervals. Annotations will be made on the global superimposed median beat. The Mortara eScribe software (or equivalent) will be used for the measurement. Interpretations for each ECG will be made by a board-certified cardiologist.

Serial blood draws for PK analysis will be obtained approximately the same time of day on Study Days 1 and 29. On those days, blood draws will be collected after each nominal ECG extraction time point. Predose blood samples on Days 1, 8, 15, and 29 should be taken within 1 hour prior to treatment administration start time. Administration may be performed by the study staff so that the subject can remain at rest.

When multiple activities are concurrent, the preferred collection order will be vital signs, safety ECGs, continuous 12-lead monitoring ECG, then PK sample.

A fasting schedule will apply for Study Days 1 and 29. Treatment administration will be under fasting conditions. A fast of at least 4 hours prior to dosing will begin before treatment on Days 1 and 29. Subjects should remain in a fasted state until completion of the 4-hour ECG and PK activities, after which a snack or light meal may be provided.

After signing consent, subjects must complete all Screening and Baseline procedures and meet all eligibility requirements to qualify for treatment. Approximately 30 eligible subjects will administer 2 mg/cm² of 0.5% w/w SNA-120 ointment to the Treatment Areas twice daily over 4 weeks to allow for evaluation of the PK with maximal use of 0.5% w/w SNA-120 ointment (proportional to approximately 35 mg active drug at each application for a subject with at least 20% BSA affected); safety and tolerability will also be assessed.

On Days 1 and 29, study assessments will be performed first, followed by the administration of study drug, which may be applied by the study staff. On Days 8 and 15, 12-lead ECGs, PK sample collection, and the study assessments will be performed first, followed by the administration of study drug, which will be applied according to instructions. Subjects will return to the research facility at Days 8, 15, and 29 for protocol-defined assessments and procedures, assessment of adverse events (AEs), and confirmation of compliance with study drug usage. A further follow-up visit is scheduled at Day 43, approximately 2 weeks after the last study drug treatment, for final pruritus, psoriasis and safety assessments.

All visit assessments of the subjects' psoriasis and associated pruritus will be made before application of study drug. Subjects should continue application of study drug for the full 4 weeks. Any subject who discontinues treatment prior to Day 29 will be asked to return to the clinic at the time of stopping study drug to undergo end-of-study assessments.

Investigational Product, Dosage and Mode of Administration: Study drug is 0.5% w/w SNA-120 ointment for topical administration.

The Affected Areas within the Treatment Area shall be treated twice daily during the entire study treatment period (Visit 2 [Study Day 1] to the end-of-treatment visit [Week 4]).

Subjects will be required to apply a thin layer (approximately 2 mg/cm²) of SNA-120 ointment to their Affected Areas twice daily during the conduct of the study covering at least 20% affected BSA. All applications should be at approximately the same time each day, approximately 12 hours apart. Prior to application, the area(s) to be treated should be clean and dry.

The investigative staff will educate subjects on the measurement and application of study ointment. The first application of ointment will be done under the supervision of the investigative site staff, at Study Day 1 (baseline), using a spatula to extract the ointment and a finger(s) to apply the ointment to the plaque. Subsequent applications will be applied at home, using the same technique as taught at the clinic.

The prescribed amount of ointment is based on the BSA affected by psoriasis (proportional to approximately 7.0 g of ointment [containing approximately 35 mg active drug] each morning and evening for subjects with 20% affected BSA). The ointment should be evenly distributed with a thin coating over all the psoriatic plaques. Before covering with clothing, the subject should wait until areas of application are dry and will not soil clothing. The investigative staff will weigh each jar of study drug before dispensing the study drug and again upon return by the subjects. On each study visit, treatment will take place after all assessments have been completed.

Duration of Participation: Up to 4 weeks of screening and 4 weeks treatment with study drug followed by a 2-week follow-up period; maximum total duration per subject of 10 weeks.

Pharmacokinetic Evaluations: Area under the plasma concentration versus time curve from time 0 to the last measurable time point (AUC₀₋ₜ); Area under the plasma concentration versus time curve from time 0 to 12 hours (AUC₀₋₁₂); Plasma concentration at 12 hours post the morning application (C₁₂ₕ); Pre-morning dose plasma concentration on days after treatment initiation (C_{trough}); Maximum plasma concentration (Cₘₐₓ): Time of maximum concentration (tₘₐₓ); Amount excreted in urine; and Renal clearance.

Safety Evaluations: Changes in ECGs, as well as incidence of QTcF prolongation; Changes in vital signs (blood pressure and pulse); Incidence and severity of all spontaneously reported AEs, including local site reactions; Changes in clinical laboratory results; Physical examinations; Concomitant medications; and Concurrent procedures.

Efficacy Evaluations: Change from baseline at each study visit in the I-NRS; Change from baseline at each study visit in the Psoriasis Area and Severity Index (PAST) score; PASI-75: response defined as a reduction of ≥75% from baseline at each study visit on the PASI; PASI-50: response defined as a reduction of ≥50% from baseline at each study visit on the PASI; Change from baseline at each study visit in the Investigator Global Assessment (IGA); Proportion of subjects with a ≥1-grade decrease on the IGA at each study visit; Proportion of subjects with a ≥2-grade decrease on the IGA at each study visit; and Change from baseline at each study visit in BSA affected by psoriasis.

Dosing Schedule: Subjects should continue to apply study drug to current active plaques, resolved/resolving plaques (residual hyper/hypopigmentation), or newly appearing plaques for the full 4 weeks of treatment, always covering a minimum of 20% BSA.

SNA-120 0.5% w/w ointment will be applied in a thin and complete layer to the Affected Areas twice daily over 4 weeks.

Approximately 2 mg/cm² SNA-120 will be applied to their psoriatic plaques twice daily during the conduct of the study. All applications should be at approximately the same time each day, approximately 12 hours apart.

The investigative staff will educate subjects on the measurement and application of study drug. Administration at the study site may be performed by the study staff so that the subject can remain at rest. All other applications will be applied at home, using the same technique as demonstrated at the study site.

Study staff should note administration start time (for predose activities) and stop time (for postdose activities).

Prior to application of study drug, the plaque area should be clean and dry. The prescribed amount of ointment, based on the BSA affected by psoriasis, should be evenly distributed over the psoriatic plaques in a thin and complete layer. The subject should not dress until areas of application are dry and will not soil clothing. The technique should be identical for each application throughout the study.

For a subject with psoriasis affecting 20% of BSA, approximately 14 g per day of ointment should be used (7 g in the morning and 7 g in the evening). Spatulas will be provided to measure ointment. One spatula measures out approximately 1 g of ointment. Each gram of the ointment should be sufficient to cover approximately 4% BSA. However, subjects should be instructed that more or less may be required and it is important that they ensure that all psoriatic lesions are completely covered with a thin, uniform, visible layer of the study drug.

The ointment contains SNA-120 (pegcantratinib [active ingredient]), butylated hydroxytoluene, benzyl alcohol, propylene glycol, white soft paraffin (white petrolatum), white wax, stearyl alcohol, and cholesterol.

Visits—There are a total of 6 scheduled visits: screening (Visit 1); treatments Visits 2 through 5 at Days 1 (baseline, 8, 15 and 29, and then, post-treatment Visit 6 at Day 43, 2 weeks after stopping treatment.

Pharmacokinetics—Blood: Serial blood draws for PK analysis will be obtained approximately the same time of day for Study Days 1 and 29. The predose blood samples on Days 1, 8, 15, and 29 should be taken within 1 h prior to treatment administration start time. On Days 1 and 29, blood draws will be collected after each nominal ECG extraction time point; the actual times of collection will be recorded in the eCRF. Administration may be performed by the study staff so that the subject can remain at rest. Single PK sample blood draws with occur on Days 8 and 15 and should be within 1 hour prior to treatment administration start time.

For all subjects, blood sampling will be performed under controlled conditions to determine the PK profile of SNA-120 from 0 to 12 hours postdose (after the end of the SNA-120 application). Blood samples will be collected at Days 1 and 29 for PK determination of SNA-120 plasma level at time=predose, and 0.5, 0.75, 1, 2, 4, 8, and 12 hours after the end of the SNA-120 application. The predose and 0.5, 0.75, and 1-hour blood samples for PK determination should be collected within 5 minutes after SNA-120 administration. The 2, 4, and 8-hour PK blood samples should be collected within 15 minutes of the nominal collection time and the 12-hour PK blood sample should be collected within 30 minutes of the nominal collection time.

Pharmacokinetics—Urine: A predose sample and a 0 to 12-hour total urine postdose sample will be collected from all subjects on Study Days 1 and 29 for PK determination of SNA-120, SNA-125, and K252b levels.

Subject Assessments—Itch Numeric Rating Scale: The I-NRS is a 1 item daily PRO measure that the subjects use to assess pruritus severity at its worst in the 24 hours prior to the assessment based on an 11-point numeric rating scale ranging from 0 (“no itching at all”) to 10 (“worst possible itching”). The I-NRS assesses overall pruritus severity. An I-NRS score of at least 4 is required to be eligible for the study. The investigator will evaluate subject eligibility based on itch associated with PV plaques at Screening. The Baseline measurement will be collected on Study Day 1 prior to study drug administration. The change from Baseline at each study visit will be determined.

Investigator Assessments: Investigator assessments include: BSA affected by psoriasis; IGA of overall psoriasis (Apremilast (Otezla) CDER Medical Review of Celgene NDA, 2014. Accessdata.fda.gov. Accessed Aug. 8, 2017.); and PASI (Fredriksson T, Pettersson U. Severe psoriasis—oral therapy with a new retinoid. Dermatologica. 1978; 157(4):238-244.).

Body Surface Area Affected by Psoriasis: Assessment of the percentage of a subject's BSA affected by psoriasis will be made by best estimates of the investigator, using the “handprint” method where 1%=1 subject handprint. At screening and baseline this should be ≥20% of BSA. The change from baseline BSA affected by psoriasis will be determined.

Investigator Global Assessment: The IGA is a site-based clinician-reported outcome measure that assesses the overall severity of psoriasis by evaluating induration (plaque elevation), erythema, and desquamation (scaling) on a 5-point scale from 0 to 4, where 0=Clear to 4=Severe. The investigator will make this assessment to calculate the overall improvement in psoriasis (the proportion of subjects achieving a rating of “Clear” or “Almost Clear” and at least a 2-point decrease [improvement] on the 5-point scale) using the IGA. The proportion of subjects achieving at least a 1-grade and 2-grade decrease from baseline will also be calculated.

Psoriasis Area and Severity Index: The PASI score is a site-based ClinRO by which a weighted sum of erythema, scaling, and induration/thickness scores over different regions of the body is determined, using the algorithm given in Fredriksson T, Pettersson U., Dermatologica. 1978; 157(4):238-244.). Each symptom will be scored on a 5-point scale from 0 to 4, where 0=Clear and 4=Very marked. The change from Baseline in the PASI score, and the percentages of subjects who have achieved a ≥75% or ≥50% reduction from baseline (PASI-75 and PASI-50, respectively), will be determined.

### Summary of Results

Study subject disposition is presented in Table 66. Demographic and baseline characteristics are summarized in Table 67. The amount and duration of study drug exposure is summarized in Table 68.

TABLE 66
Subject Disposition

| Category | N (%) | |
|---|---|---|
| Enrolled | 30 | |
| Treated | 30 | (100) |
| Completed Study (Visit 6) | 27 | (90) |
| Discontinued | 3 | (10) |
| Discontinued due to AE | 0 | (0) |

TABLE 67
Subject Demographics and Baseline Characteristics

| Characteristic | | N = 30 | |
|---|---|---|---|
| Age (years) | mean (SD) | 45.8 | (10.1) |
| Female | n (%) | 11 | (36.7) |
| Race - White | n (%) | 28 | (93.3) |
| BMI (kg/m²) | mean (SD) | 34.3 | (8.1) |
| Percent BSA | mean (SD) | 30.1 | (10.5) |
| PASI | mean (SD) | 18.72 | (6.86) |
| IGA | | | |
| Moderate (3) | n (%) | 23 | (76.7) |
| Severe (4) | n (%) | 7 | (23.3) |
| NRS | mean (SD) | 8.3 | (1.3) |

TABLE 68
Study Drug Exposure

| SNA-120 (N = 30) | Mean (SD) | |
|---|---|---|
| Duration of exposure (days) | 28.0 | (3.9) |
| Total amount of study drug applied (g) | 830.43 | (585.36) |

Pharmacokinetic Results: On Days 1 and 29, the majority of subjects had no detectable levels of SNA-120 in the plasma (66.7% and 67.9% of subjects, respectively). 10 and 9 out of 30 and 28 subjects (33.3% and 32.1%, respectively on Days 1 and 29) had detectable levels of SNA-120 in the plasma. Nearly all were lower than 2.5 ng/mL (i.e., <1 nM), except for 1 subject who had a Cₘₐₓ of 5.04 ng/mL (i.e., 2.05 nM) on Day 1 and 4.64 ng/mL (i.e., 1.89 nM) on Day 29, respectively. Overall, there was no accumulation of SNA-120 in the plasma following repeated BID topical application. In all post-baseline PK sampling timepoints, the maximum number of subjects with a detectable level of SNA-120 was 6 (out of 30). Of 489 post-baseline blood PK samples, only 65 (13.3%) had detectable levels of SNA-120 (>0.500 ng/mL). In addition, there were no detectable plasma levels of the amide form of SNA-120 (<1.00 ng/mL) or K252b (<0.250 ng/mL), the two potential metabolites of SNA-120, at any post-baseline timepoint. PK results are summarized in Table 69.

TABLE 69
SA-120 Plasma Concentrations at Days 1 and 29

| | Peak Plasma SNA-120 Concentrations (ng/mL) | | |
|---|---|---|---|
| | Below Quantitation | <2.50 (2.5 ng/mL | |
| | Limit (BQL; i.e., | is approximately | |
| | 0.500 ng/mL plasma) | 1 nM) | ≥2.50 |
| Day 1 | <0.500 | 0.500-1.51 | 5.04 |
| (N = 30) | (n = 20; 66.7%) | (n = 9; 30.0%) | (n = 1; 3.3%) |
| Day 29 | <0.500 | 0.583-0.902 | 4.64 |
| (N = 28) | (n = 19; 67.9%) | (n = 8; 28.6%) | (n = 1; 3.6%) |

ECG Including QTcF Assessment: The number of subjects undergoing standard ECG recordings at each pre-specified post-dosing time point varied from 27 to 29, and the number of subjects with Holter-extracted ECGs ranged from 22 to 29. The standard ECG recording results are summarized in Table 70. The observed mean changes from baseline were small. No trends over time were present. No individual changes were clinically meaningful. Results from the Holter-extracted ECGs are shown in Table 71. Changes from baseline during Days 1 and 29 were generally small and consistent with circadian and spontaneous variation. No dose-response pattern was present. No individual changes were clinically meaningful. Overall, no subjects in this study demonstrated a QTcF duration >500 msec or an increase from baseline of >60 msec. In this study of 30 patients with on-treatment ECG recordings at up to 15 pre-specified time points on multiple days and within-day sampling, there was no evidence of an ECG effect of 0.5% (w/w) SNA-120.

TABLE 70
Standard ECG Results: Minimum and Maximum Mean Change

| | | | | |
|---|---|---|---|---|
| | Minimum | | Maximum | |
| ECG | mean (SD) | Day | Mean (SD) | Day |

| | | | | | |
|---|---|---|---|---|---|
| HR, bpm | −0.1 | (9.9) | 43 | 3.1 (11.3) | 8 |
| PR, msec | −1.5 | (12.8) | 15 | 2.4 (13.5) | 8 |
| QRS, msec | −0.5 | (6.0) | 15 | 4.0 (6.6) | 43 |
| QTcF, msec | 1.7 | (23.6) | 15 | 9.1 (16.4) | 43 |

TABLE 71
Holter ECG Results - Minimum and Maximum Mean Change
from Baseline Assessed Over 12 Hours on Days 1 and 29

| | Minimum | | | Maximum | |
|---|---|---|---|---|---|
| ECG | mean (SD) | | Day/Hour | Mean (SD) | Day/Hour |
| HR, bpm | −5.12 | (6.23) | 1/1 | 8.14 (5.68) | 1/12 |
| | −4.25 | (7.80) | 29/1 | 9.50 (9.73) | 29/8 |
| PR, msec | −1.31 | (8.08) | 1/12 | 2.33 (6.66) | 1/0.75 |
| | 0.39 | (10.84) | 29/12 | 3.90 (8.61) | 29/0.5 |
| QRS, msec | −1.32 | (4.95) | 1/8 | 1.13 (3.85) | 1/0.75 |
| | −1.90 | (4.75) | 29/8 | 1.46 (5.23) | 29/1 |
| QTcF, msec | −5.12 | (11.32) | 1/12 | 2.93 (9.63) | 1/2 |
| | −4.97 | (13.96) | 29/12 | 5.98 (10.45) | 29/0.75 |

Adverse Events: A total of 9 subjects reported 12 AEs, one of which (i.e., mild pruritus) was deemed related to study drug. No subject reported a severe AE. There were no serious AEs (SAES) and no deaths reported during the study. No subject discontinued the study due to an AE. Table 72 summarizes AEs.

TABLE 72
Adverse Events

| | N = 30 |
|---|---|
| Type of Event | n (%) [events] |
| AE | 9 (30) [12] |
| Severe AE | 0 (0) |
| SAE | 0 (0) |
| AE related to study drug | 1 (3.3) [1] |
| AE resulting in discontinuation | 0 (0) |

Exploratory Efficacy: This study enrolled moderate to severe psoriasis patients with >20% BSA and at least moderate pruritus (NRS >4) (e.g., moderate to severe). At baseline, subjects had a mean PASI score of 18.72 and a mean itch-NRS of 8.3. Subjects were treated twice daily with 0.5% SNA-120 (w/w) ointment in an open label protocol. After 4 weeks of treatment, subjects experienced a mean improvement from baseline in itch-NRS of 3.4 (or 41.35%). Sixty percent of subjects had at least a 3-grade improvement in itch-NRS. After 4 weeks of treatment, subjects experienced a mean improvement from baseline in PASI of 6.44. Ten (33.3%) and 3 (10%) of the subjects achieved PASI 50 and PASI 75, respectively. With respect to IGA, 30% and 13.3% of subjects experienced at least a 1-grade and 2-grade improvement, respectively, from baseline to 4 weeks. Efficacy results are summarized in Table 73.

TABLE 73
Exploratory Efficacy Results

| Endpoint | | Week 4 | |
|---|---|---|---|
| NRS | mean (SD) | −3.4 | (2.4%) |
| (change from baseline) | | | |
| NRS | mean (SD) | −41.35 | (29.30%) |
| (% change from baseline) | | | |
| NRS 50 | n (%) | 14 | (46.7%) |
| NRS 3-grade | n (%) | 18 | (60.0%) |
| PASI | mean (SD) | −6.44 | (7.35%) |
| (change from baseline) | | | |
| PASI 50 | n (%) | 10 | (33.3%) |
| PASI 75 | n (%) | 3 | (10.0%) |
| IGA 1-grade | n (%) | 9 | (30%) |
| IGA 2-grade | n (%) | 4 | (13.3%) |

Conclusions: These results support the hypotheses described in the study protocol regarding the PK and safety profiles of SNA-120. There was minimal to no systemic exposure after topical application of SNA-120 under maximal use conditions and no associated clinically significant ECG changes, including no evidence of QTc prolongation. There were no systemic safety signals and no dermal tolerability issues. Additionally, improvements in pruritus and psoriasis were observed in exploratory efficacy analyses.

### Example 10: Randomized, Double-Blind, Placebo-Controlled, Phase 2B, Multicenter Study to Evaluate the Safety, Efficacy, and Tolerability of SNA-120 (Pegcantratinib) Ointment in Male and Female Subjects with Pruritus Associated with Psoriasis Vulgaris

The study subjects (mild-to-moderate psoriasis patients with at least moderate pruritus (itch)) consisted of 208 adult subjects that were ≥18 years of age, randomized 1:1:1 to SNA-120 0.05%, SNA-120 0.5% and vehicle (see Table 1). Subjects self-administered the assigned study drug (SNA-120 ointment or vehicle; the vehicle formulation was the same as the SNA-120 formulation without the active) to the indicated areas twice-daily over 12 weeks. The ointment contains SNA-120 (pegcantratinib [active ingredient]), butylated hydroxytoluene, benzyl alcohol, propylene glycol, white soft paraffin (white petrolatum), white wax, stearyl alcohol, and cholesterol (see Table 1). Assessments were performed to determine the treatment effect on pruritus, as well as the underlying visible plaque psoriasis, and safety and local tolerability of SNA-120. Pruritus was assessed by the subject-administered I-NRS and recorded on their daily diaries. Plaque psoriasis was assessed by the investigator by Investigator Global Assessment (IGA) and Psoriasis Area Severity Index (PAST). At baseline, subjects were required to have a weekly mean I-NRS score of at least 5 on the 11 point I-NRS scale, and mild to moderate plaque psoriasis (IGA 2 or 3).

Investigator Global Assessment: The IGA is a site-based (clinician-reported outcome measure) that assesses the overall severity of psoriasis by evaluating induration (plaque elevation), erythema, and desquamation (scaling) on a 5-point scale from 0 to 4, where 0=Clear to 4=Severe. The investigator will make this assessment to calculate the overall improvement in psoriasis (the proportion of subjects achieving a rating of “Clear” or “Almost Clear” and at least a 2-point decrease [improvement] on the 5-point scale) using the IGA. The proportion of subjects achieving at least a 1-grade and 2-grade decrease from baseline will also be calculated.

Psoriasis Area and Severity Index: The PASI score is a site-based investigator assessed (clinician reported outcome measure) by which a weighted sum of erythema, scaling, and induration/thickness scores over different regions of the body is determined, using the algorithm given in Fredriksson T, Pettersson U., Dermatologica, 1978, 157(4):238-244). Each symptom will be scored on a 5-point scale from 0 to 4, where 0=Clear and 4=Very marked. The change from Baseline in the PASI score, and the percentages of subjects who have achieved a ≥75% or ≥50% reduction from baseline (PASI-75 and PASI-50, respectively), will be determined.

As seen in Table 74, SNA-120 0.05% demonstrated a statistically significant effect on two key measures of psoriasis, PASI-75 and IGA ≥2-grade improvement with clear (grade 0) or almost clear (grade 1). SNA-120 ointment was well tolerated and demonstrated an acceptable safety profile based on the treatment-emergent averse events, the majority of which were mild to moderate.

As seen in Table 75, there was a reduction from baseline to the study Week 8 (primary endpoint) in the week's mean (±SD) daily pruritus score (Itch Numeric Rating Scale [I-NRS]; the I-NRS is an 11-point scale ranging from ‘no itch’ (0) to the ‘worst imaginable itch’ (10) that study participants used daily to report the intensity of their worst itch in the previous 24 hours) of −4.3±2.41 (p=0.244) for SNA-120 ointment 0.05%, −3.7±2.34 (p=0.499) for SNA-120 ointment 0.5%, and −4.0±2.57 for vehicle. The I-NRS reduction of ≥3 point and ≥4 point were observed for 45±72.6% (p=0.251) and 33±53.2% (p=0.557), 37±57.8% (p=0.545) and 28±43.8% (p=0.097), and 41±63.1% and 38±58.5% of subjects for SNA-120 0.05%, SNA-120 0.5%, and vehicle, respectively, at Week 8.

SNA-120 was well-tolerated with no serious treatment-related adverse events. In this study, subjects treated with SNA-120 (0.05%) achieved statistical significance, compared to vehicle, on endpoints of psoriasis disease severity. Patients treated with SNA-120 (0.05%) experienced a mean 4.3 point (58%) reduction from baseline on the I-NRS, compared to a mean 4.0 point (55%) reduction with vehicle (not statistically significant, p=0.244). SNA-120 (0.5%) showed similar results. Subjects treated with SNA-120 also experienced a meaningful reduction in itch; the result did not reach statistical significance against placebo (i.e., subjects treated with vehicle lacking SNA-120 experienced a meaningful reduction in itch).

On pre-specified, secondary endpoints related to the clearance of plaques, SNA-120 (0.05%) demonstrated statistically significant and clinically meaningful improvement. Specifically, 27% of subjects experienced a 75% reduction in their Psoriasis Area and Severity Index (PAST 75) score from baseline, compared to 13% of subjects treated with vehicle (p=0.045). The study also included an Investigator Global Assessment (IGA), in which 29% of patients achieved a two-grade improvement and ‘clear’ or ‘almost clear’ skin, compared to 13% of subjects treated with vehicle (p=0.036). Both the PASI 75 and IGA results remained statistically significant at 14 weeks, two weeks after discontinuation of treatment.

TABLE 74

| | | |
|---|---|---|
| Treatment Group | Week 8 | Week 12 |

IGA ≥2 grade and 0/1 score (proportion of subjects) (p value)

| | | |
|---|---|---|
| SNA-120 0.05% | 10 (16.4%) | 17 (28.8%) |
| | p = 0.518 | p = 0.036 |
| Vehicle | 8 (12.5%) | 8 (13.3%) |

PASI-75 (proportion of subjects) (p value)

| | | |
|---|---|---|
| SNA-120 0.05% | 15 (24.6%) | 16 (27.1%) |
| | p = 0.063 | p = 0.045 |
| Vehicle | 8 (12.5%) | 8 (13.3%) |

TABLE 75

| | | | | |
|---|---|---|---|---|
| I-NRS | | | Week 8 | Week |
| Measure | Baseline | Week 1 | (Primary) | 12(EoT) |

Mean (SD); CFB (p value)

| SNA-120 | 7.3 (1.31) | 5.7 | (1.53) | 3.0 | (1.96) | 2.6 | (2.10) |
|---|---|---|---|---|---|---|---|
| 0.05% | | −1.6 | (1.51) | −4.3 | (2.41) | −4.6 | (2.44) |
| | | P = 0.212 | | P = 0.244 | | P = 0.231 | |
| SNA-120 | 7.4 (1.24) | 5.8 | (1.63) | 3.8 | (2.24) | 3.5 | (2.5) |
| 0.5% | | −1.6 | (1.43) | −3.7 | (2.34) | −3.9 | (2.61) |
| | | P = 0.423 | | P = 0.499 | | P = 0.384 | |
| Vehicle | 7.4 (1.39) | 6.0 | (1.78) | 3.4 | (2.63) | 3.0 | (2.56) |
| | | −1.3 | (1.53) | −4.0 | (2.57) | −4.3 | (2.60) |

% of subjects with ≥3 point improvement (p value)

| SNA-120 | NA | 9 | (12.9) | 45 | (72.6) | 47 | (79.7) |
|---|---|---|---|---|---|---|---|
| 0.05% | | P = 0.602 | | P = 0.251 | | P = 0.297 | |
| SNA-120 | NA | 10 | (14.5) | 37 | (57.8) | 36 | (63.2) |
| 0.5% | | P = 0.811 | | P = 0.545 | | P = 0.300 | |
| Vehicle | NA | 11 | (15.9) | 41 | (63.1) | 44 | (72.1) |

% of subjects with ≥4 point improvement (p value)

| SNA-120 | NA | 5 | (7.1) | 33 | (53.2) | 34 | (57.6) |
|---|---|---|---|---|---|---|---|
| 0.05% | | P = 0.256 | | P = 0.557 | | P = 0.890 | |
| SNA-120 | NA | 5 | (7.2) | 28 | (43.8) | 32 | (56.1) |
| 0.5% | | P = 0.244 | | P = 0.097 | | P = 0.750 | |
| Vehicle | NA | 2 | (2.9) | 38 | (58.5) | 36 | (59.0) |

What is claimed is:

1. A formulation for dermal delivery of polymer conjugates of a polymer conjugate of an indolocarbazole compound with reduced exposure, the formulation comprising:
the polymer conjugate of an indolocarbazole compound;
a preservative/antioxidant;
a preservative/solvent;
a solvent;
a solvent/moisturizing agent;
a stiffening agent; and
one or more emollients/emulsifying agents.

2. The formulation of claim 1, wherein the polymer conjugate of an indolocarbazole compound is SNA-120.

3. The formulation of claim 2, wherein the SNA-120 is present at about 0.005% to about 5% (w/w) of the formulation.

4. The formulation of claim 2, wherein the SNA-120 is present at about 0.05% to about 0.5% (w/w) of the formulation.

5. The formulation of claim 1, wherein:
the preservative/antioxidant is ascorbic acid, tocopherol, propyl gallate, tertiary butylhydroquinone (TBHQ), butylated hydroxyanisole (BHA), or butylated hydroxytoluene (BHT);
the preservative/solvent is benzoic acid, sorbic acid, boric acid, methylparaben, ethylparaben, propylparaben, butylparaben, sodium benzoate, sodium propionate, potassium sorbate, chlorobutanol, benzyl alcohol and phenyl ethyl alcohol, phenol, chlorocresol, o-phenyl phenol, benzalkonium chloride, cetyl pyridinium chloride, imidurea, thimerisal, propyl gallate, butylated hydroxyanisole (BHA), butylated hydroxytoluene (BHT), citric acid, or disodium edetate;
the solvent is ethanol, propylene glycol, glycerin, or polyethylene glycol;
the solvent/moisturizing agent is mineral oil, white petrolatum, stearyl alcohol, cetyl alcohol, isopropyl myristate, diisopropyl adipate, stearic acid, or white wax;
the stiffening agent is white wax, dimethicone, or a polymer; and
the one or more emollient/emulsifying agent is mineral oil, petrolatum, cholesterol, dimethicone, dimethiconol, stearyl alcohol, cetyl alcohol, behenyl alcohol, diisopropyl adipate, isopropyl myristate, myristyl myristate, cetyl ricinoleate, sorbitan distearate, sorbitan dilaurate, sorbitan stearate, sorbitan laurate, sucrose laurate, sucrose dilaurate, sodium isostearyl lactylate, lauryl pidolate, sorbitan stearate, PPG-14 butyl ether, PPG-15 stearyl ether, or a combination thereof.

6. (canceled)

7. (canceled)

8. The formulation of claim 1, wherein:
the preservative/antioxidant is butylated hydroxytoluene (BHT);
the preservative/solvent is benzyl alcohol;
the solvent is propylene glycol;
the solvent/moisturizing agent is white petrolatum; and
the stiffening agent is white wax; and
the one or more emollients/emulsifying agents is a combination of stearyl alcohol and cholesterol.

9. The formulation of claim 1, wherein the preservative/antioxidant is present at about 0.01% to about 1% (w/w) of the formulation.

10. The formulation of claim 1, wherein the preservative/antioxidant is present at about 0.10% (w/w) of the formulation.

11. (canceled)

12. (canceled)

13. The formulation of claim 1, wherein the preservative/solvent is present at about 1% to about 10% (w/w) of the formulation.

14. The formulation of claim 1, wherein the preservative/solvent is present at about 5% (w/w) of the formulation.

15. (canceled)

16. (canceled)

17. The formulation of claim 1, wherein the solvent is present at about 0.5% to about 20% (w/w) of the formulation.

18. The formulation of claim 1, wherein the solvent is present at about 5% to about 6% (w/w) of the formulation.

19. (canceled)

20. (canceled)

21. The formulation of claim 1, wherein the solvent/moisturizing agent is present at about 10% to about 95% (w/w) of the formulation.

22. The formulation of claim 1, wherein the solvent/moisturizing agent is present at about 75% (w/w) of the formulation.

23. (canceled)

24. (canceled)

25. The formulation of claim 1, wherein the stiffening agent is present at about 0.5% to about 20% (w/w) of the formulation.

26. The formulation of claim 1, wherein the stiffening agent is present at about 8% (w/w) of the formulation.

27. (canceled)

28. The formulation of claim 1, wherein the one or more emollients/emulsifying agents is a combination of two emollients/emulsifying agents is used.

29. The formulation of claim 1, wherein the one or more emollients/emulsifying agents is a combination of stearyl alcohol and cholesterol.

30. The formulation of claim 1, wherein the one or more emollients/emulsifying agents are present at about 0.5% to about 10% (w/w) of the formulation.

31. The formulation of claim 29, wherein both the stearyl alcohol and cholesterol are present at about 3% (w/w) of the formulation.

32. An ointment for dermal delivery of SNA-120, comprising:
about 0.05% to about 0.5% w/w SNA-120;
about 0.1% w/w butylated hydroxytoluene (BHT);
about 5% w/w benzyl alcohol;
about 5% to about 6% w/w propylene glycol;
about 75% w/w white petrolatum;
about 8% w/w white wax;
about 3% w/w stearyl alcohol; and
about 3% w/w cholesterol.

33. A method of treating a skin condition associated with TrkA signalling in a subject in need thereof, comprising:
applying a topical formulation to a skin region of the subject, wherein the topical formulation inhibits TrkA signaling in the skin region, thereby treating the skin condition;
wherein the topical formulation comprises:
a polymer conjugate of an indolocarbazole compound;
a preservative/antioxidant;
a preservative/solvent;
a solvent;
a solvent/moisturizing agent;
a stiffening agent; and
one or more emollients/emulsifying agents.

34. The method of claim 33, wherein the polymer conjugate of an indolocarbazole is SNA-120.

35. The method of claim 33, wherein the skin condition is psoriasis.

36. The method of claim 33, wherein the skin condition comprises pruritus associated with psoriasis or another dermatologic condition.
